(19) United States
(12) Patent Application Publication
(10) Pub. No.: US 2026/0315477 A1
(43) Pub. Date: Oct. 15, 2026

(54) THERAPEUTIC APPROACH TO BLOCK TERMINAL EXHAUSTION OF T CELLS AND SYNERGIZETHE EFFICACY OF T CELL IMMUNOTHERAPIES

(71) Applicant: Ohio State Innovation Foundation, Columbus, OH (US)

(72) Inventor: Hazem GHONEIM, Dublin, OH (US)

(21) Appl. No.: 19/483,031

(22) PCT Filed: May 9, 2024
(86) PCT No.: PCT/US2024/028547

§ 371 Date: Nov. 10, 2025

Related U.S. Application Data
(60) Provisional application No. 63/465,169, filed on May 9, 2023.
(60) Provisional application No. 63/635,878, filed on Apr. 18, 2024.
(60) Provisional application No. 63/642,350, filed on May 3, 2024.

(51) Int. Cl. A61K 31/519; A61K 31/44; A61P 35/00; A61P 37/04; C07K 16/28

(52) U.S. Cl. CPC A61K 31/519; A61K 31/44; A61P 35/00; A61P 37/04; C07K 16/2818; C07K 16/2827; C07K 2317/76

(57) ABSTRACT

The present disclosure provides for a method of preventing T-cell terminal exhaustion comprising contacting the T-cell with a histone demethylase inhibitor. Further provided herein is a method of improving CAR-T therapy effectiveness, a method of treating cancer comprising, a method of treating a chronic virus infection, a method of improving immune activity in a subject, and a method of treating a disease or disorder.

### CROSS-REFERENCE TO RELATED APPLICATIONS

This application claims the benefit of U.S. Provisional Application No. 63/465,169 filed on May 9, 2023, U.S. Provisional Application No. 63/635,878 filed on Apr. 18, 2024, and U.S. Provisional Application No. 63/642,350 filed on May 3, 2024, each of which is hereby incorporated by reference herein in its entirety.

### BACKGROUND

Cytotoxic CD8 T lymphocytes—an important defense against tumors or virus-infected cells—progressively lose their killing function and become exhausted during cancer or chronic virus infections. Terminal exhaustion of T cells remains a major challenge to T cell immunotherapy. In some cases, this barrier can be surmounted with immune checkpoint blockade (ICB), which rejuvenates the less-exhausted T cells by blocking signals from inhibitory receptors. Despite the success of ICB in treating some cancers, many patients remain nonresponsive. One need in T cell immunotherapy is the development of terminal exhaustion state.

The methods disclosed herein address these and other needs.

### SUMMARY

In accordance with the purposes of the disclosed materials and methods, as embodied and broadly described herein, the disclosed subject matter, in one aspect, relates to T-cell terminal exhaustion.

Thus, in one example, a method of preventing T-cell terminal exhaustion is provided, including contacting the T-cell with a histone demethylase inhibitor.

In a further example, a method of improving CAR-T therapy effectiveness is provided, including (1) contacting a genetically engineered CAR-T-cell with a histone demethylase inhibitor; and (2) administering the CAR-T-cell to a subject in need thereof.

Additionally, a method of improving CAR-T therapy effectiveness is provided, including (1) administering a genetically engineered CAR-T-cell to a subject in need thereof, and (2) administering a histone demethylase inhibitor to a subject in need thereof.

In an example, a method of treating cancer is provided, including administering to a subject in need thereof, a histone demethylase inhibitor.

In a further example, a method of treating a chronic virus infection is provided, including administering to a subject in need thereof, a histone demethylase inhibitor.

Additionally, a method of improving immune activity in a subject in need thereof is provided, including administering to the subject a histone demethylase inhibitor.

In one example, a method of treating a disease or disorder is provided, including administering to a subject in need thereof a histone demethylase inhibitor.

Additional advantages will be set forth in part in the description that follows, and in part will be obvious from the description, or may be learned by practice of the aspects described below.

The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1 shows a schematic diagram of the demethylation of a CD8 T cell, upon which it is established that the loss of H3K4me3 marks at effector/memory-related genes enables de novo DNA methylation that enforces terminal exhaustion of CD8 T cells and that targeting H3K4 demethylases in CD8 T cells blocks their progression towards terminal exhaustion and enhances ICB response.

FIGS. 2A-2B shows blocking de novo DNA methylation skews the open chromatin landscape of chronically stimulated CD8 T cells toward the functional memory state. Multiple-line plot of the chromatin accessibility changes in effector (FIG. 2A) and stemness-associated programs in naïve, effector (day 8), exhausted WT, chronically stimulated cKO (day 35), and memory (day 35) antigen-specific CD8 T cells after chronic or acute LCMV infection (FIG. 2B).

FIG. 3 shows that terminally exhausted T cells lose H3K4me3 marks and undergo de novo DNA methylation coupled with reduced chromatin accessibility at effector/memory loci. Snapshots of the histone H3K4me3 CUT&Tag-seq within exhausted T cell subsets (GSE149810); the WGBS (GSE99450) and ATAC-seq datasets (unpublished) showing DNA methylation and open chromatin peaks, respectively, within naïve and chronically stimulated antigen-specific Dnmt3a-cKO or WT CD8 T cells; and open chromatin peaks within the Tprog subset (PD-1+Cxcr5+Tim-3−) or PD-1+Tim-3+ antigen-specific CD8 T cells (GSE132110) during chronic LCMV infection.

FIGS. 4A-4F show that terminally exhausted CD8 T cells upregulate H3K4me3 demethylases. Plots and heatmap showing transcript levels of H3K4 demethylases within naïve or LCMV-specific TEX cell subsets (4A, 4E: GSE100745; 4B, 4F: GSE149879). (4C) Interaction plot showing average KDM5B transcript levels in effector (day 7), acutely stimulated (Acute TCR), chronically stimulated (Ch.TCR), or Dysfunctional human CD8 T cells on day 28 (GSE217072). (4D) Heatmap showing promoters with variable H3K4me3 levels among TEX subsets during chronic LCMV infection (GSE149810).

FIGS. 5A-5C show blocking de novo DNA methylation in CD8 T cells preserves the rejuvenation potential after stopping ICB therapy. Anti-PD-L1 treatment in chronically infected WT and Dnmt3a cKO mice. (FIG. 5A) FACS plots of Ki67 levels (FIG. 5B) and Fold change of proliferating LCMV-specific CD8 T cells 1 month after anti-PD-L1 therapy (FIG. 5C). N>4 mice/group, 3 independent exps. *P<0.05, **P<0.01, ***P<0.001 (Mann-Whitney U test). Error bars=SEM

FIGS. 6A-6G shows targeting KDM5A and KDM5B enzymes reactivates effector functions of terminally dysfunctional human CD8 T cells. Experimental design of in vitro model of human T cell dysfunction (FIG. 6A). Bar graphs showing frequencies of IFNG+CD8 T cells (FIG. 6B), and expression levels of CD107a (FIG. 6C), TNFa (FIG. 6D), Tbet (FIG. 6E), and TCF1 (FIG. 6F) in human CD8 T cells on day 28 under chronic stimulation settings. Numbers of dead AML cells after 18 hr of coculture with human CD8 T cells (FIG. 6G). *P-value<0.05, **<0.01, ****<0.0001 (One-way ANOVA test, Tukey). Error bars indicate SEM. N=3 replica of 2-3 independent experiments.

FIGS. 7A-7E show that targeting KDM5A and KDM5B enhances TEX responses to ICB during chronic LCMV infection. Treatment regimen of KDM5A/Bi (GSK467) and anti-PD-L1 of chronic LCMV-infected mice (FIG. 7A). Bar graphs showing numbers of P14 CD8 T cells (FIG. 7B), polyclonal LCMV-specific CD8 T cells (FIG. 7C), Tprog cells (PD-1+Cx3cr1−Tim3−) (FIG. 7D), and Cytolytic (PD-1+Cx3cr1+) CD8 T cells (FIG. 7E) in spleens after treatments. N=4 mice/group, 2 independent exps *P-value<0.05, **<0.01 (Mann-Whitney U test). Error bars=SEM.

FIG. 8 shows the timeline for experimentation.

FIGS. 9A-9B show the epigenetic reprogramming of CD8 T cells during virus infection or cancer. FIG. 9A shows a schematic regarding the reprogramming of exhausted T cell to functional T cell. FIG. 9B shows a schematic regarding the invitro model of human T cell dysfunction.

FIGS. 10A-10D show that terminally exhausted T cells lose H3K4me3 permissive marks at effector/memory genes (Chen et al., Nat Imm 2021) for TBX21 genes (FIG. 10A), Tcf7 genes (FIG. 10B), Cx3cr1 genes (FIG. 10C), and IL7R genes (FIG. 10D).

FIGS. 11A-111D show that cytolytic exhausted T cells downregulate KDM5B and maintain high H3K4me3 levels at effector genes (Chen et al. Net Imm 2021). FIG. 11A shows KDM5B levels in mouse CD8 T cells. FIG. 11B shows LS mean in human CD8 T cells. FIG. 11C shows variable H3K4me3 signals at promoter regions in Tcyto vs. Tterm cells. FIG. 11D shows the change in KDM5B and H3K4me3 for Stem-like cells, cytolytic cells, and terminally exhausted cells.

FIGS. 12A-12F show that targeting KDM5A and KDM5B enhance effector function and memory programs in dysfunctional human CD8 T cells (KDM5B=H3K4me3 demethylase). FIG. 12A shows % INFG+CD8 T cells. FIG. 12B shows IFNG-MFI IFNG+CD8T cells. FIG. 12C shows TNFa-MF1 IFNG+CD8 T cells. FIG. 12D shows CD107A-MF1 IFNG+CD8 T cells. FIG. 12E shows TBET-MFI IFNG+CD8 T cells. FIG. 12F shows the timeline of human CDMCs based on phenotypic and functional analysis of CD8 T cells.

FIGS. 13A-13B show that targeting KDM5A and KDM5B recovers cytotoxic activity within dysfunctional human CD8 T cells. FIG. 13A shows anti-tumor data on day 28. FIG. 13B shows the number of dead tumor cells per 100 CD8 T cells for varying T cells.

FIGS. 14A-14F show that therapeutic blockade of KDM5A and KDM5B boosts the response of exhausted anti-viral CD8 T cells to ICB therapy. FIG. 14A shows the timeline for a chronic LCMV infection model. FIG. 14B shows data for LCMV-specific CD8 T cells. FIG. 14C shows date for GP33-specific P14 CD8 T cells. FIG. 14D shows data for “Stem-Like” CD8 T cells. FIG. 14E shows data for cytolytic CD8 T cells. FIG. 14F shows data for terminally exhausted CD8 T cells.

FIGS. 15A-15C show therapeutic blockade KDM5A and KDM5B boosts the responses of exhausted anti-viral CD8 T cells to ICB therapy. FIG. 15A shows the #of IFNg+CD8 T cells for IFNg+PD-1+CD8 T cells. FIG. 15B shows TNFa MFI of CD44+PD-1+CD8 T cells for TNFa MFI, FIG. 15C shows Tbet MFI of CD44+PD-1+CD8 T cells for Tbet MFI.

FIGS. 16A-16C show therapeutic blockade of KDM5A and KDM5B boosts the responses of anti-tumor CD8 T cells to ICB therapy. FIG. 16A shows the timeline of the breast adenocarcinoma model. FIG. 16B shows tumor volume of a PYMT-OVA tumor over time. FIG. 16C shows the number of varying T cells for a KDM5A/B inhibitor and aPD-L1 and mock+aPD-L1.

FIG. 17 shows that therapeutic blockade of KDM5A/B boosts the responses of anti-tumor CD8 T cells to ICB therapy.

FIG. 18 shows that KDM5B shows pre-tumorigenic by promoting immune evasion.

FIG. 19 shows a schematic diagram of KDM5A/B inhibitor, GSK-467, conjugating with anti-PD-1 as administered to an exhausted T-cell.

FIGS. 20A-20F show that terminally exhausted CD8 T cells upregulate H3K4me3 demethylases. FIG. 20A shows a heatmap showing promoters with H3K4me3 level among TEX subsets during chronic LCMV infection (GSE 149810). FIGS. 20B-20F show heatmaps and plots showing RNA levels of H3k4 demethylases within murine naïve or LCMV-specific TEX cell subsets (B-D: PRJNA497086; B, E, F: GSE149879).

FIGS. 21A-21D show that dysfunctional tumor-reactive human CD8 T cells upregulate KDM5B. FIG. 22A shows heatmaps showing transcript levels of dysfunction/memory-associated genes, and H3K4 demethylases within tumor-infiltrating human CD8 T cell subsets in breast cancer (GSE110686) or (FIG. 22B) brain metastases from melanoma patients. FIG. 22C shows interaction plots showing average KDM5A or (FIG. 22D) KDM5B transcript levels in effector (day 7), acutely stimulated (Acute TCR), chronically stimulated (Ch.TCR) or dysfunctional human CD8 T cells on day 28 (GSE217072).

FIGS. 22A-22D show that CRISPR-Cas9 gene editing of chronically stimulated human CD8 T cells. FIG. 22A shows a schematic for ex vivo stimulation of human CBMCs by anti-CD3/CD28, followed by Cas9 editing of target genes on day 7 using Cas9-RNP approach. After overnight resting, chronic TCR+TGF(31 stimulation is continued. FIG. 22B shows a histogram and bar graph showing expression levels of TCRab protein on control or TRAC-edited human CD8 T cells. FIG. 22C shows a schematic for Cas9-lentiviral vector transduction approach to KO DNMT3A in ex vivo stimulated human CD8 T cells. FIG. 22D shows that following resting and selection of transduced CD8 T cells (day7-8), percent polyfunctional chronically stimulated human CD8 T cells after DNMT3A gene-editing was measured. N=3 wells per group, 2 experiments. *P<0.05 Welch's test.

FIGS. 23A-23C show the experimental design of in vivo studies.

FIG. 24 shows the experimental design of an in vivo study involving phenotypic, functional, and epigenetic analyses of adoptively transferred Thy1.1+CD8 T cells.

FIGS. 25A-25B show in vivo studies for B16-gp33 tumor cells (FIG. 25A) and PyMT-OVA tumor cells (FIG. 25B).

FIGS. 26A-26B show that the selective KDM5A/B inhibitor treatment increases B16 melanoma tumor death in vitro. FIG. 26A shows line graphs showing frequencies of dead B16-F10-GP33. FIG. 26B shows PyMT-OVA tumor cells after 24, 48, and 72 hours of GSK467 (KDM5B/Ai) treatment using different doses in vitro. Ordinary two-way ANOVA test, Dunnett's test. Error bars indicate SEM. N=4 replica of 2 independent experiments.

FIGS. 27A-27B show animal studies for chronic LCMV infection (FIG. 27A) and CT2A-gp33 tumor cells (FIG. 27B).

FIG. 28 shows a schematic of epigenetic reprogramming of exhausted CD 8 T cells by targeting histone-DNA methylation crosstalk.

FIGS. 29A-29B shows that histone modifications regulate de novo DNA methylation.

FIG. 29A shows post-translational histone modifications. FIG. 29B shows pathways for H3K4me3 and H3K4me0.

FIG. 30 shows that loss of H3K4me3 is coupled with de novo DNA methylation in terminally TEX cells.

FIGS. 31A-31E show that the therapeutic inhibition of KDM5A/B-mediated H3K4 demethylation enhances effector functions of human TEX cells with data of dysfunctional human CD8 T cells collected on Day 28 of experimentation.

FIGS. 32A-32E show that the therapeutic inhibition of KDM5A/B-mediated H3K4 demethylation enhances effector functions of human TEX cells with data of dysfunctional human CD8 T cells collected on Day 28 of experimentation.

FIGS. 33A-33B show that the therapeutic inhibition of KDM5A/B-mediated H3K4 demethylation enhances effector functions of human TEX cells on day 28 of experimentation.

FIG. 34 shows example Oxford nanopore sequencing technology for analyzing the targeting of DNA methylation in human CD8 T cells.

FIG. 35 shows the percent average CpG methylation from 7 to 28 days.

FIGS. 36A-36E show that the therapeutic blockade of H3K4 demethylation boosts the responses of anti-viral CD8 T cells to ICB.

FIGS. 37A-37B show that the therapeutic blockade of H3K4 demethylation boosts the responses of anti-viral CD8 T cells to ICB based on a melanoma model.

FIGS. 38A-38C show that the therapeutic blockade of H3K4 demethylation boosts the responses of anti-viral CD8 T cells to ICB based on a glioma tumor model.

FIGS. 39A-39D show that the therapeutic blockade of H3K4 demethylation boosts the responses of anti-viral CD8 T cells to ICB based on a glioma tumor model for tumor specific CD8 T cells (tumors).

FIGS. 40A-40D show that the therapeutic blockade of H3K4 demethylation blocks the progression to terminal exhaustion. FIG. 40A shows the experimental timeline for chronic LCMV infection. FIG. 40B shows the experimental timeline for 5K naïve Thy1.1+/1.2+P14 cells. FIG. 40C shows the distribution of A.T. progenitor TEX cells for mock+aPD-L1. FIG. 40D shows the distribution of A.T. progenitor TEX cells for H3K4 demethylase inhibitor+aPD-L1.

FIG. 41 shows a schematic depicting the targeting of H3K4 demethylation to enhance CD8 T cell responses to ICB.

FIG. 42 shows demethylase enzymes that remove H3K4 methylation marks. KDM1A “LSD1” removes H3K4me2, mel1. KDM1B “LSD2” removes H3K4me2, me1. KDM5A removes H3K4me3, me2. KDM5B removes H3K4me3, me2, me1. KDM5C is X chromosome-linked and removes H3K4me3, me2. KDm5D is Y chromosome-linked and removes H3K4me3, me2.

FIG. 43 shows that terminal dysfunction in human CD8 T cells is epigenetically stabilized based on tracking changes in phenotype and function of CD8 T cells.

FIG. 44 shows a schematic of the genetic targeting of histone H3K4 demethylases in human CD8 T cells via CRISPR-Cas9 RNP approach.

FIGS. 45A-45F show that targeting the H3K4 demethylases KDM5A and KDM5B enhances the effector function of chronically stimulated human CD8 T cells based on chronic TCR. Summary bar graphs and representative FACs plots showing frequencies of polyfunctional IFNγ+TNF+CD8+ T cells, and non-cytokine secreting (IFNγ− TNF−) CD8+ T cells after PMA/Ionomycin stimulation (day 14). Values are mean±SEM; One-way ANOVA, *p-value<0.05, **p<0.01, ***p<0.001, ****p<0.0001. FIGS. 45A-45D show flow cytometry plots for non-targeting sgRNA ctrl (A) KDM5B KO (B) KDM5A KO (C) and KDM5A+KDM5B KO (D). FIGS. 45E-45F shows percent IFNg+TNFa+CD8+ T cells for IFNγ and TNF co-producing “polyfunctional” (E) and non-cytokine-secreting “severely exhausted” (F).

FIGS. 46A-46F show that targeting the H3K4 demethylases KDM5A and KDM5B enhances the effector function of chronically stimulated human CD8 T cells based on chronic TGFB1+TCR (dysfunctional). Summary bar graphs and representative FACs plots showing frequencies of polyfunctional IFNγ+TNF+CD8+ T cells, and non-cytokine secreting (IFNγ− TNF−) CD8+ T cells after PMA/Ionomycin stimulation (day 14). Values are mean±SEM; One-way ANOVA, *p-value<0.05, **p<0.01, ***p<0.001, ****p<0.0001. FIGS. 46A-46D show flow cytometry plots for non-targeting sgRNA ctrl (A) KDM5B KO (B) KDM5A KO (C) and KDM5A+KDM5B KO (D). FIGS. 46E-46F shows percent IFNg+TNFa+CD8+ T cells for IFNγ and TNF co-producing “polyfunctional” (E) and non-cytokine-secreting “severely exhausted” (F).

FIGS. 47A-47E show that genetic targeting of KDM5A and KDM5B boosts the cytotoxic and degranulation activities and enhances proliferation capacity of dysfunctional human CD8 T cells.

FIG. 48 shows small molecule inhibitors of KDM5A and KDM5B histone demethylases.

FIGS. 49A-49E show that the therapeutic inhibition of KDM5A/B-mediated H3K4 demethylation enhances effector functions of human TEX cells via testing of GSK467 and C70, both KDM5A/B inhibitors.

FIGS. 50A-50D show that therapeutic inhibition of KDM1A (LSD1) does not enhance effector functions of human TEX cells, where FIG. 50A shows percent total IFNγ+CD8 T cells, FIG. 50B shows percent IFNγ+CD107a+CD8 T cells, percent IFNγ+TNF+CD8 T cells, and CD107-MFI in IFNγ+CD8 Tcells.

FIGS. 51A-51C show that therapeutic blockade of H3K4 demethylase boost the response of anti-viral CD8 T cells to ICB, wherein FIG. 51A show data for progenitor CD8 T cells, FIG. 51B shows data for cytolytic CD8 T cells, and FIG. 51C shows data for IFNg+PD-1+CD8 T cells.

FIG. 52 shows DNA demethylation transcription for an activated gene and a repressed gene.

FIG. 53 shows a schematic of de novo DNA methylation, as mediated by Dnmt3a, and its impact on the development of terminal exhaustion.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of.” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. Furthermore, prevent refers to the lowering of an event or characteristic (e.g., tumor growth). It is understood that this is typically in relation to some standard or expected value, in other words it is relative, but that it is not always necessary for the standard or relative value to be referred to. For example, “prevent T-cell terminal exhaustion” means reducing the rate of terminal exhaustion relative to a standard or a control.

The term “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

### Methods

De novo DNA methylation, mediated by the Dnmt3a enzyme, silences T cell function and enforces terminal exhaustion, thereby restraining the efficacy of ICB therapy. However, key regulators of de novo epigenetic programs that govern T cell dysfunction have previously remained largely unknown.

Proposed herein is a therapeutic approach to reverse progression towards terminal exhaustion by harnessing crosstalk among histone-DNA epigenetic mechanisms in CD8 T cells. Discussed herein, to investigate these mechanisms, is a novel in vitro model that establishes an epigenetically stabilized, terminal dysfunction in human CD8 T cells. The role of the activating histone H3K4me3 marks in regulating CD8 T cell (dys)function.

Loss of H3K4me3 marks at effector/memory genes enables de novo DNA methylation programming that enforces terminal exhaustion of CD8 T cells. However, therapeutic targeting of the H3K4 demethylase, KDM5B, in CD8 T cells blocks their progression towards terminal exhaustion, enhancing their effector function and rejuvenation response to immunotherapies.

Targeting the H3K4 demethylase, KDM5B, enzyme in dysfunctional human T cells improved their effector function and cytotoxicity in vitro. In addition, during chronic LCMV infection, combined treatment by a small molecule selective inhibitor and anti-PD-L1 (ICB) significantly enhanced the ICB responses of stem-like and cytolytic subsets of exhausted T cells. The combined therapy resulted in a significant tumor control in two different murine models of cancer, including breast adenocarcinoma and glioma. These findings provide mechanistic insights for developing therapeutic approaches to epigenetically reprogram exhausted T cells and enhance T cell immunotherapies, including CAR T cell therapy.

The present disclosure, in one aspect, provides for a method of preventing T-cell terminal exhaustion comprising contacting the T-cell with a histone demethylase inhibitor.

T-cell exhaustion refers to various epigenetic and metabolic states of post-thymic T cells. In some examples, it refers to T cells that have a reduced capacity to secrete cytokines and increased expression of inhibitory receptors. In further examples, exhaustion is characterized by stepwise ad progressive loss of T cell functions, which can ultimately result in physical deletion of the responding cells. Exhaustion can occur following or during chronic viral infections as well as in cancer, particularly with tumor growth. Terminally exhausted T cells are terminally differentiated cells. De novo DNA demethylation contributes to the terminal exhaustion of T cells.

Histone demethylases are enzymes that remove methyl groups from histones, targeting methylated cites for demethylation. Histone demethylase inhibitors inhibit these enzymes, thereby inhibiting the removal of methyl groups from histones.

In some examples, the method comprises contacting the T-cell in vivo in a subject in need thereof. Contacting in vivo includes, but is not limited to, administration orally, intravenously, intradermally, intrathecally, parenterally, nasally, via intramuscular injection, rectally, subcutaneously, intravaginally, ocularly, via inhalation, via mucous membrane, or any combination thereof.

In further examples, the method comprises contacting the T-cell ex vivo. Contacting the T-cells ex vivo includes contacting the T-cells with the histone demethylase inhibitor in a laboratory setting, wherein the T-cells are collected from a subject that is later administered the altered T-cells or from another subject to then administer to the present subject.

In some examples, the histone demethylase inhibitor comprises an inhibitor of H3K4me3. H34me3 is an epigenetic modification to the DNA packaging protein histone H3, which indicates tri-methylation at the 4^{th} lysine residue of the histone H3 protein. It denotes the addition of three methyl groups to the lysine 4 of the histone H3 protein. In some examples, H3K4me3 is associated with the activation of transcription of nearby genes via the recruitment of PHD-domain-containing proteins involved in transcription initiation. H3K4me3 inhibitors inhibit the methylation caused by H3K4me3 in the transcription process.

In further examples, H3K4me3 comprises KDM5A enzyme, KDM5B enzyme, or any combination thereof.

The histone demethylases of the KDM5 family are mediators of gene expression dynamics during developmental, cellular differentiation, and other nuclear processes. There are four human members of the KDM5 family, two of which are KDM5A and KDM5B, both of which are part of a larger group of JumonjiC domain containing, 2-oxyglutarate and Fe(II)-dependent dioxygenases. KDM5A is considered a histone demethylase subfamily member and erases di- and tri-methyl groups from lysine 4 of histone H3. KDM5B is a protein coding gene and is related to pathways of transcription and RNE polymerase. During differentiation, KDM5A represses cell cycle genes and promotes cell cycle exit, while KDM5B inhibits cell cycle exit.

In some examples, H3K4me3 comprises KDM1A enzyme, KDM1B enzyme, or any combination thereof. KDM1A is a protein encoding a nuclear protein containing a SWIRM domain, a FAD-binding motif, and an amine oxidase domain. KDM1A is a component of several histone deacetylase complexes but can silence genes by functioning as a histone demethylase. KDM1B is a flavin-dependent histone demethylase and regulates histone lysine methylation, thereby regulating gene expression and chromatin function.

In further examples, the inhibitor of H3K4me3 comprises JIB-04, CPI455, QC6352, AS8351, KDOAM-25 citrate, KDM5A-IN-1, KDM5-IN-1, TK-129, YUKA1, JQKD82 trihydrochloride, CPI-455 hydrochloride, KDM5-C49, JQKD82, GSK467, C70, or any combination thereof.

These inhibitors inhibit either of KDM5A or KDM5B, while some inhibit both KDM5A and KDM5B.

In some examples, the inhibitor of H3K4me3 comprises ladademstat, GSK2879552, tranylcypromine hemisulfate, pulrodemstat benzenesulfonate, seclidemstat, bomedemstat dihydrochloride, GSK 690 hydrochloride, S2101, INCB059872 dihydrochloride, HDAC6-IN-3, LSD1-IN-30, CBB1007 trihydrochloride, INCB059872, CBB1007 hydrochloride, LSD1-IN-6, LSD1/HDAC6-IN-1, MC2652, LSD1-UM-109, 51427, LSD1-IN-23, LSD1/2-IN-4, NCL1, cis-4-Br-2,5-F2-PCPA, KDM1/CDK1-IN-1, LSD1-IN-26, LSD1-IN-21, LSD1-IN-18, LSD1-IN-25, arborinine, LSD1-IN-22, LSD1-IN-19, LSD1/2-IN-3, tranylcypromine-d5 hydrochloride, LSD1/ER-IN-1, CBB1003 hydrochloride, GSK467, C70, or any combination thereof.

These inhibitors inhibit either of KDM1A or KDM1B, while some inhibit both KDM1A and KDM1B.

In certain examples, the inhibitor of H3K4me3 comprises GSK467, C70, or any combination thereof. GSK467 is a cell penetrant and selective inhibitor of KDM5A and KDM5B, with a Kᵢ of 10 nM and an IC₅₀ of 26 nM. It shows 180-fold selectivity for KDM4C and no measurable inhibitory effects toward KDM6 or other Jumonji family members. GSK467 has the following chemical structure:

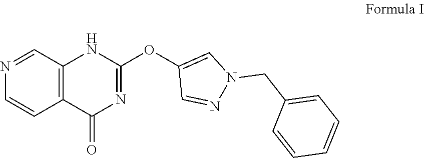 [SMILES: O=c1nc(Oc2cnn(Cc3ccccc3)c2)[nH]c2cnccc12]

C70 is an ethyl ester derivative of KDM5-C49 and a potent, cell-permeable and KDM5A and KDM5B histone demethylase inhibitor. C70 has an antiproliferative effect in myeloma cells, leading to genome-wide elevation of H3K4me3 levels. C70 has the following chemical structure:

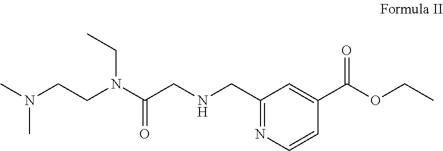 [SMILES: CCOC(=O)c1ccnc(CNCC(=O)N(CC)CCN(C)C)c1]

In specific examples, the T-cell is a CD8 T-cell. T cells are a type of white blood cell called a lymphocyte. CD8 T cells are one type of T cell wherein the T cell has a CD8 receptor on its membrane. CD8 T cells kill cells that are infected with viruses and bacteria, a well as tumor cells. CD8 T cells are effector cells and recognize antigens and release cytotoxic granules, such as perforin and granzyme B, which directly lyse infected or transformed cells.

Further provided herein is a method of improving CAR-T therapy effectiveness comprising: (1) contacting a genetically engineered CAR-T-cell with a histone demethylase inhibitor; and (2) administering the CAR-T-cell to a subject in need thereof CAR-T therapy is a type of treatment in which a subject's T cells are modified in the laboratory so as to attack cancer cells. The T cells are taken from a subject's blood and then genetically modified to add the chimeric antigen receptor (CAR) gene, wherein this receptor binds to a certain protein on cancer cells. A large number of the CAR-T cells are grown I the laboratory and then given to the subject via infusion.

Genetically engineered CAR-T cell refers to T-cells that are genetically engineered such that they comprise the gene for the chimeric antigen receptor antigen, which allows for binding to cancer cells.

In some examples, the genetically engineered CAR-T-cell is contacted with the histone demethylase inhibitor ex vivo.

In further examples, the genetically engineered CAR-T-cell is contacted with the histone demethylase inhibitor in vivo in a subject in need thereof.

Also provided herein is a method of improving CAR-T therapy effectiveness comprising: (1) administering a genetically engineered CAR-T-cell to a subject in need thereof, and (2) administering a histone demethylase inhibitor to a subject in need thereof.

Administering a histone demethylase inhibitor includes administration orally, intravenously, intradermally, intrathecally, parenterally, nasally, via intramuscular injection, rectally, subcutaneously, intravaginally, ocularly, via inhalation, via mucous membrane, or any combination thereof.

In some examples, the histone demethylase inhibitor comprise an H3K4me3 inhibitor.

In further examples, H3K4me3 comprises KDM5A enzyme, KDM5B enzyme, or any combination thereof.

In certain examples, H3K4me3 comprises KDM1A enzyme, KDM1B enzyme, or any combination thereof.

In specific examples, the inhibitor of H3K4me3 comprises JIB-04, CPI455, QC6352, AS8351, KDOAM-25 citrate, KDM5A-IN-1, KDM5-IN-1, TK-129, YUKA1, JQKD82 trihydrochloride, CPI-455 hydrochloride, KDM5-C49, JQKD82, GSK467, C70, or any combination thereof.

In some examples, the inhibitor of H3K4me3 comprises ladademstat, GSK2879552, tranylcypromine hemisulfate, pulrodemstat benzenesulfonate, seclidemstat, bomedemstat dihydrochloride, GSK 690 hydrochloride, S2101, INCB059872 dihydrochloride, HDAC6-IN-3, LSD1-IN-30, CBB1007 trihydrochloride, INCB059872, CBB1007 hydrochloride, LSD1-IN-6, LSD1/HDAC6-IN-1, MC2652, LSD1-UM-109, S1427, LSD1-IN-23, LSD1/2-IN-4, NCL1, cis-4-Br-2,5-F2-PCPA, KDM1/CDK1-IN-1, LSD1-IN-26, LSD1-IN-21, LSD1-IN-18, LSD1-IN-25, arborinine, LSD1-IN-22, LSD1-IN-19, LSD1/2-IN-3, tranylcypromine-d5 hydrochloride, LSD1/ER-IN-1, CBB1003 hydrochloride, GSK467, C70, or any combination thereof.

In further examples, the inhibitor of H3K4me3 comprises GSK467, C70, or any combination thereof.

In specific examples, the T-cell is a CD8 T-cell.

Further provided herein is a method of treating cancer comprising administering to a subject in need thereof, a histone demethylase inhibitor.

Types of cancer include, but are not limited to, bladder cancer, breast cancer, cervical cancer, colorectal cancer, gynecological cancer, head and neck cancer, kidney cancer, liver cancer, lung cancer, mesothelioma, myeloma, ovarian cancer, prostate cancer, skin cancer, uterine cancer, and vaginal and vulvar cancers.

Also provided herein is a method of treating a chronic virus infection comprising administering to a subject in need thereof, a histone demethylase inhibitor.

Chronic viral infections are characterized by the continued presence of an infectious virus following the primary infection. In some examples, this includes chronic or recurrent disease. In further examples, this includes slow infection which is characterized by a prolonged incubation period followed by progressive disease.

Further provided herein is a method of improving immune activity in a subject in need thereof, comprising administering to the subject a histone demethylase inhibitor.

In some examples, the method further comprises administering an immune checkpoint inhibitor.

Immune checkpoint inhibitor refers to immunotherapy drugs that work by blocking checkpoint proteins from binding with their partner proteins. They prevent signals from being sent between partner proteins thereby allowing the T cells to kill cancer cells.

In further examples, administering to the subject in need thereof a histone demethylase inhibitor comprises contacting a T-cell ex vivo and administering to the subject the T-cell.

In certain examples, administering to the subject in need thereof a histone demethylase inhibitor comprises contacting a T-cell in vivo in the subject in need thereof.

In specific examples, the histone demethylase inhibitor comprises an inhibitor of H3K4me3.

In some examples, the cancer comprises breast adenocarcinoma or glioma.

Breast adenocarcinoma refers to a type of breast cancer wherein the cancer starts in the epithelial cells of the breast and forms tumors. More specifically, adenocarcinomas form from epithelial cells in the breast ducts (milk ducts) or lobules (glands in the breast that make milk).

Glioma is a growth of cells that starts in the brain or spinal cord in the glial cells that surround and support neurons in the brain, including astrocytes, oligodendrocytes, and ependymal cells. As the glioma grows and forms a mass of cells, it becomes a tumor. Gliomas are referred to as intra-axial brain tumors because they grow within the substance of the brain and often mix with normal brain tissue. About 33 percent of all brain tumors are gliomas. Gliomas include astrocytomas, brain stem gliomas, ependymomas, oligo-astrocytomas, oligodendrogliomas, and optic pathway gliomas.

In further examples, the immune checkpoint inhibitor inhibits PD-1, PD-L1, CTLA-4, LAG3, or any combination thereof.

PD-1 is a protein found on T cells and helps in controlling a subject's immune system. PD-1 immune checkpoint inhibitors inhibit PD-i's ability to control the subject's immune system thereby increasing the ability of T cells to kill cancer cells. In some examples, PD-1 binds to another protein, PD-L1, thereby keeping T cells from killing other cells, including cancer cells.

PD-L1 is a protein that aids in controlling a subject's immune response. In some examples, PD-L1 is found on non-cancerous cells, and in further examples, PD-L1 is found on cancer cells in higher-than-normal amounts. PD-L1 immune checkpoint inhibitors inhibit PD-L1's ability to control the subject's immune system thereby increasing the ability of T cells to kill cancer cells.

CTLA-4 is a protein found on T cells that contributes to controlling a subject's immune system. When CTLA-4 is bound to another protein called B7, it keeps T cells from killing other cells, which can include cancer cells. CTLA-4 immune checkpoint inhibitors inhibit CTLA-4's ability to control the subject's immune system thereby increasing the ability of T cells to kill cancer cells.

LAG-3 is a protein found on T cells and helps in controlling a subject's immune system. LAG-3 immune checkpoint inhibitors inhibit LAG-3's ability to control the subject's immune system thereby increasing the ability of T cells to kill cancer cells.

In some examples, breast adenocarcinoma and glioma are treated with surgery, radiation therapy, chemotherapy, or any combination thereof, in addition to the administration of a histone demethylase inhibitor, and in some examples administration of an immune checkpoint inhibitor, as disclosed herein.

In certain examples, the immune checkpoint inhibitor inhibiting PD-1 comprises nivolumab, pembrolizumab, cemiplimab, dostarlimab, retifanlimab, toripalimab, or any combination thereof.

In specific examples, the immune checkpoint inhibitor inhibiting PD-L1 comprises atezolizumab, avelumab, durvalumab, or any combination thereof.

In some examples, the immune checkpoint inhibitor inhibiting CTLA-4 comprises ipilimumab, tremelimumab, or any combination thereof.

In further examples, the immune checkpoint inhibitor inhibiting LAG-3 comprises eftilagimod alpha, relatlimab, favezolimab, fianlimab, or any combination thereof.

In certain examples, H3K4me3 comprises KDM5A enzyme, KDM5B enzyme, or any combination thereof.

In specific examples, H3K4me3 comprises KDM1A enzyme, KDM1B enzyme, or any combination thereof.

In some examples, the inhibitor of H3K4me3 comprises JIB-04, CPI455, QC6352, AS8351, KDOAM-25 citrate, KDM5A-IN-1, KDM5-IN-1, TK-129, YUKA1, JQKD82 trihydrochloride, CPI-455 hydrochloride, KDM5-C49, JQKD82, GSK467, C70, or any combination thereof.

In further examples, the inhibitor of H3K4me3 comprises ladademstat, GSK2879552, tranylcypromine hemisulfate, pulrodemstat benzenesulfonate, seclidemstat, bomedemstat dihydrochloride, GSK 690 hydrochloride, S2101, INCB059872 dihydrochloride, HDAC6-IN-3, LSD1-IN-30, CBB1007 trihydrochloride, INCB059872, CBB1007 hydrochloride, LSD1-IN-6, LSD1/HDAC6-IN-1, MC2652, LSD1-UM-109, S1427, LSD1-IN-23, LSD1/2-IN-4, NCL1, cis-4-Br-2,5-F2-PCPA, KDM1/CDK1-IN-1, LSD1-IN-26, LSD1-IN-21, LSD1-IN-18, LSD1-IN-25, arborinine, LSD1-IN-22, LSD1-IN-19, LSD1/2-IN-3, tranylcypromine-d5 hydrochloride, LSD1/ER-IN-1, CBB1003 hydrochloride, GSK467, C70, or any combination thereof.

In certain examples, the inhibitor of H3K4me3 comprises GSK467, C70, or any combination thereof.

In specific examples, the T-cell is a CD8 T-cell

In some examples, the chronic virus infection comprises acquired immune deficiency syndrome (AIDS), AIDS-related complexes, chronic hepatitis, long COVID, subacute sclerosing panencephalitis (chronic measles encephalitis), chronic papovavirus encephalitis (progressive multifocal leukoencephalopathy), spongioform encephalopathies, herpesvirus-induced diseases, neoplasis, or any combination thereof.

Also provided herein is a method of treating a disease or disorder comprising administering to a subject in need thereof a histone demethylase inhibitor.

Administration of a histone demethylase inhibitor can be accomplished by any of the methods discussed herein. The disease and/or disorders can include those disclosed herein as well as others not disclosed herein. Histone demethylase inhibitor can be administered alone as the sole method of treating a disease/disorder in a subject or as part of a multiple treatments, including but not limited to CAR-T therapy or a series of varying drugs. Treating a disease and/or disorder can including reducing the prevalence of the disease/disorder, reducing the rate of spread, growth or severity of the disease/disorder, preventing further spread, growth, or worsening of the disease/disorder, eliminating the disease/disorder, and/or reducing the subject's ability to transmit the disease/disorder to other subjects.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Targeting Epigenetic Crosstalk that Restrains the Responses of Exhausted T Cells to Immune Checkpoint Blockade Therapy

### Introduction

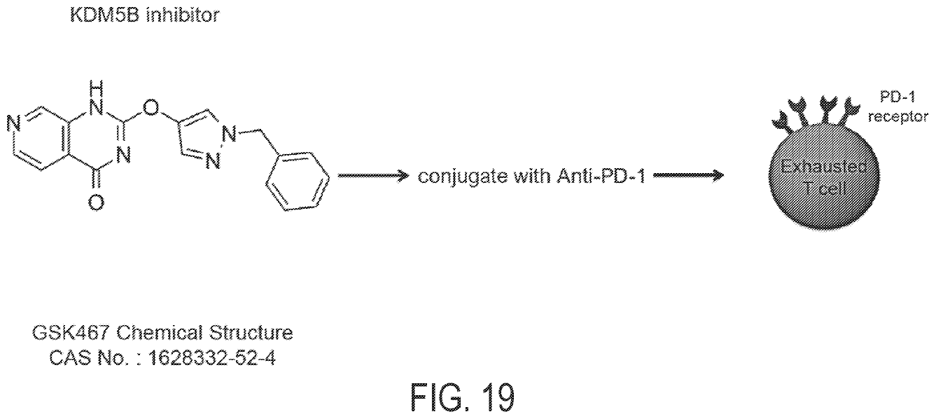

Epigenetic scarring of exhausted CD8 T (TEX) cells during chronic virus infections or cancer remains a major cell-intrinsic barrier to T cell immunotherapies, including immune checkpoint blockade (ICB). For successful epigenetic reprogramming of TEX cells, it is useful to identify and target the molecular mechanisms underlying terminal exhaustion. De novo DNA methylation enforces silencing of T cell function and restrains their responses to ICB therapy. Discussed herein is whether post-translational histone modifications crosstalk with de novo DNA methylation during the progression to terminal exhaustion. To better understand the interplay between these epigenetic mechanisms in chronically stimulated CD8 T cells, the novel in vitro model of human T cell dysfunction, as well as preclinical models of T cell exhaustion including chronic LCMV infection. There is a relationship between dynamic histone changes and de novo DNA methylation in TEX cells. Targeting a key histone-modifying enzyme in dysfunctional human T cells improved their effector function and cytotoxicity in vitro. In addition, during chronic LCMV infection, combined treatment by a selective inhibitor and anti-PD-L1 significantly enhanced the ICB responses of stem-like and cytolytic subsets of TEX cells. FIG. 19. Further explored herein is the impact of targeting histone modifications on DNA methylation programming in CD8 T cells. These findings provide important mechanistic insights for developing novel therapeutic approaches to epigenetically reprogram TEX cells and enhance T cell immunotherapies.

Cytotoxic CD8 T lymphocytes provide adaptive defense against tumors or virus-infected cells. However, under prolonged antigen exposure, CD8 T cells lose their killing function and become exhausted, failing to eliminate cancer or chronic viral infections. T cell exhaustion occurs progressively transitioning through three major subsets: (a)progenitor (Tprog) cells that act as a chronic resource for other subsets, (b) a highly cytolytic subset (Tcyto), and (c) terminally-exhausted (Tterm) cells that lose the effector functions. (McLane (2019); Hudson (2019)) Immune checkpoint blockade (ICB) is a cancer immunotherapy that reinvigorates the Tprog and Tcyto subsets by blocking signals from inhibitory receptors (e.g., CTLA-4, PD-1). Yet, Tterm cells remain refractory to ICB, perhaps explaining why many patients whose anti-tumor T cells are terminally-exhausted remain nonresponsive. (Sharma (2020)) As such, the goal herein was to understand how terminal exhaustion is acquired in T cells, and to block or reverse this process to enhance the efficacy of T cell immunotherapies.

De novo DNA methylation, mediated by the Dnmt3a enzyme, silences T cell function and enforces terminal exhaustion, thereby restraining the efficacy of ICB therapy 4. However, key regulators of de novo epigenetic programs that govern T cell dysfunction have previously remained largely unknown. Proposed herein is an approach to reverse progression towards terminal exhaustion by harnessing crosstalk among epigenetic mechanisms in CD8 T cells. To investigate these mechanisms, a novel in vitro model was developed that establishes an epigenetically stabilized, terminal dysfunction in human CD8 T cells. (Saadey (2022)) Preliminary data revealed an important role of the activating H3K4me3 marks in regulating CD8 T cell (dys)function. Based on these data, it was hypothesized that loss of H3K4me3 marks at effector/memory genes enables de novo DNA methylation programming that enforces terminal exhaustion of CD8 T cells. However, therapeutic targeting of the H3K4 demethylase, KDM5B, in CD8 T cells blocks their progression towards terminal exhaustion, enhancing the ICB response. The role of H3K4me3 modifications in regulating exhaustion-associated de novo DNA methylation programs.

H3K4me3 marks are more enriched in Tcyto cells than in Tterm cells, the H3K4 histone demethylase, KDM5B, is downregulated in the Tcyto subset but maintained in Tterm cells, and H3K4me3 enrichment inhibits Dnmt3a-mediated DNA methylation activity. (Saadey (2022)); Chen (2021); Guo (2015)). It remained unknown whether the loss of H3K4me3 modifications primed chronically stimulated CD8 T cells for terminal exhaustion by enabling de novo DNA methylation programming. To better understand this epigenetic crosstalk, the tractable in vitro model of human T cell dysfunction was employed and a complementary in vivo model of chronic LCMV infection. (Saadey (2022)) Profile dynamic changes were profiled in H3K4me3 levels and DNA methylation programs as CD8 T cells progressed towards terminal dysfunction. The integrative analyses of these epigenetic changes allowed for determining whether: de novo DNA methylation tracks with an initial loss of H3K4me3 in Tterm cells; and downregulating KDM5B in chronically stimulated CD8 T cells preserves H3K4me3 marks and hinders exhaustion-associated DNA methylation.

These immuno-epigenetic studies reveal if an interplay between histone H3K4me3 and de novo DNA methylation programming orchestrates terminal exhaustion of CD8 T cells. Targeting KDM5B in TEX cells enhances their ICB responses by curbing epigenetic progression towards terminal exhaustion.

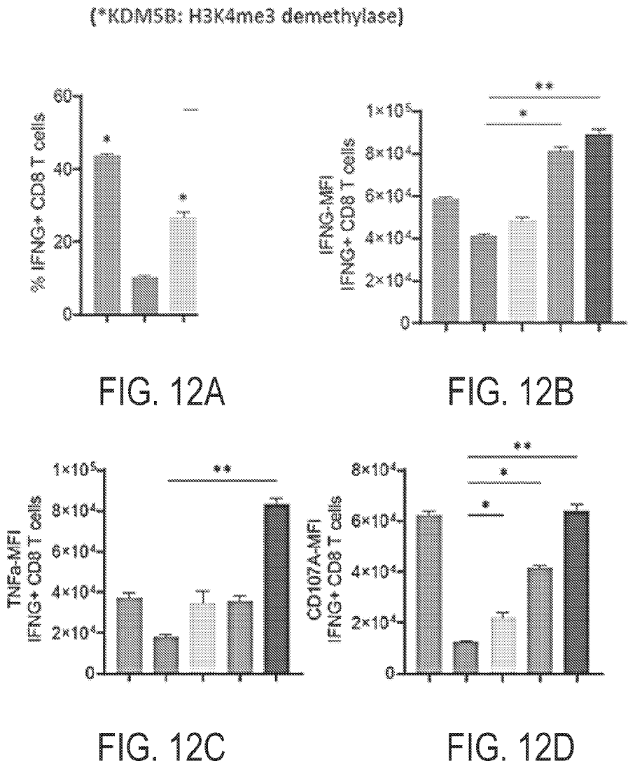

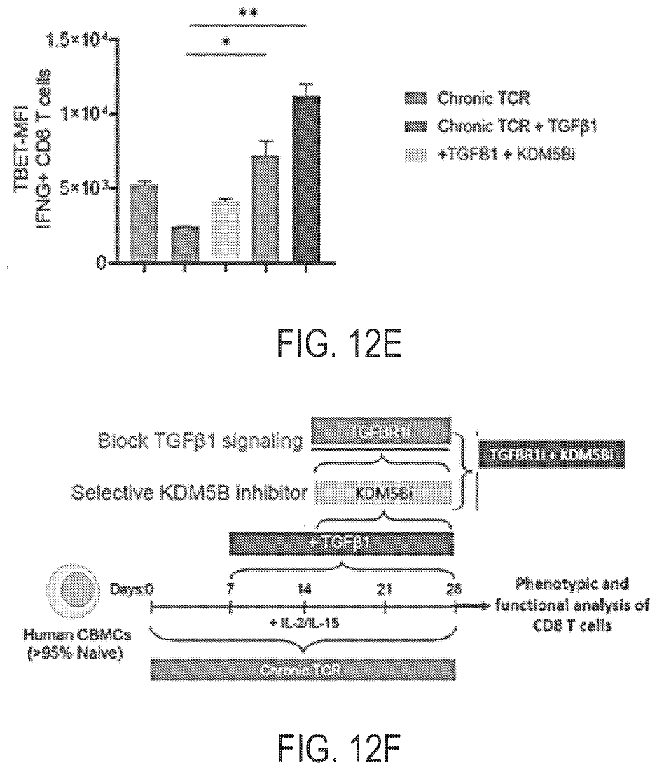

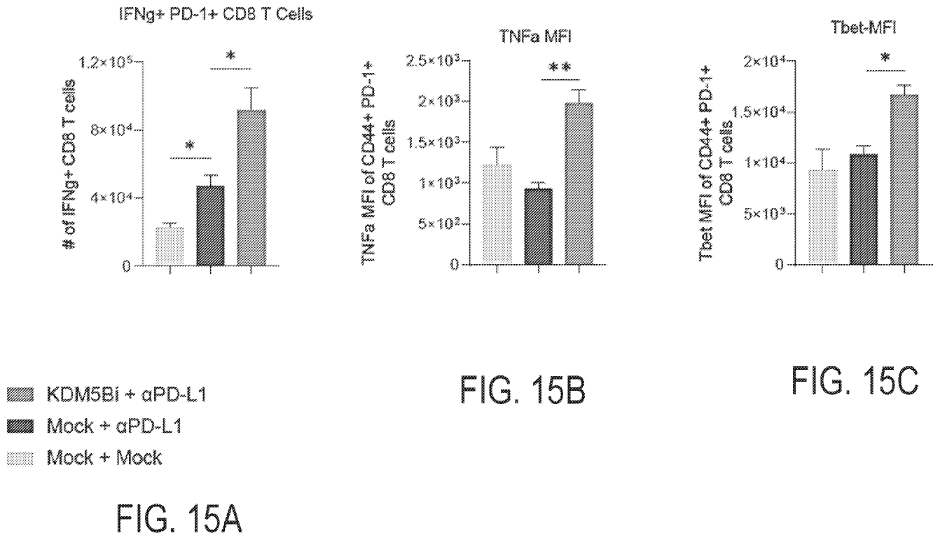

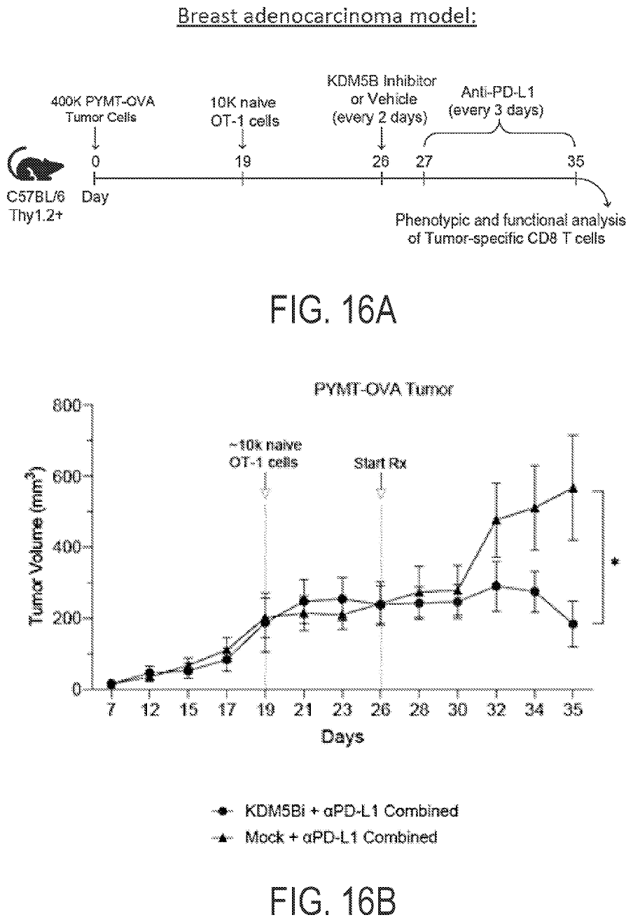

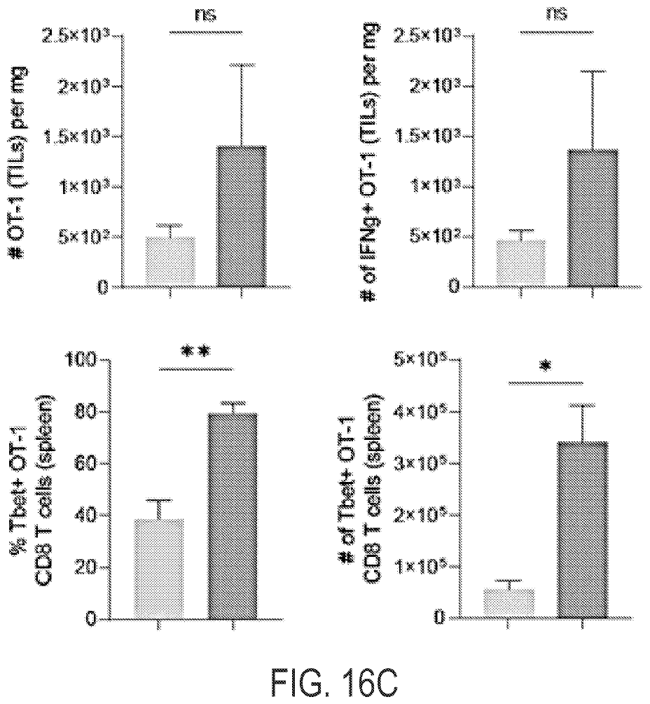

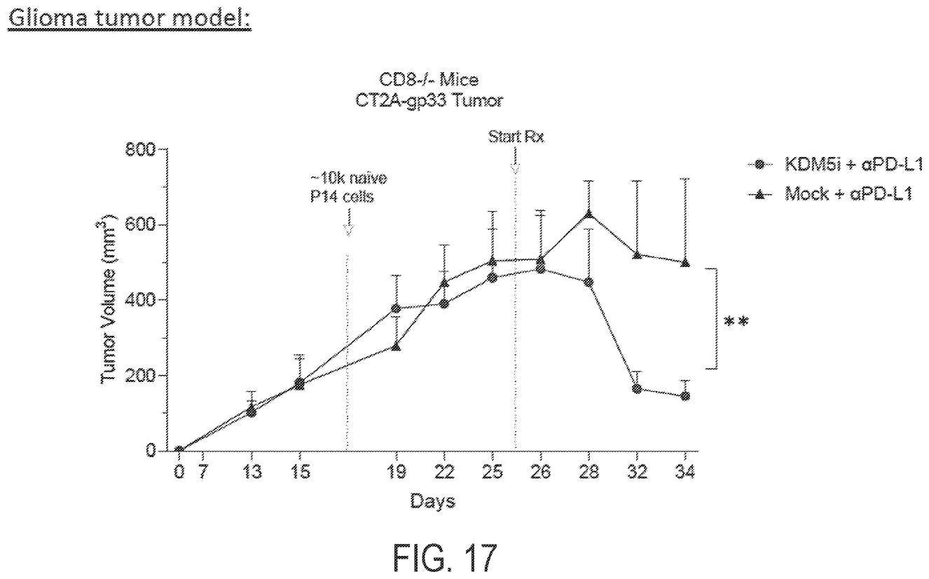

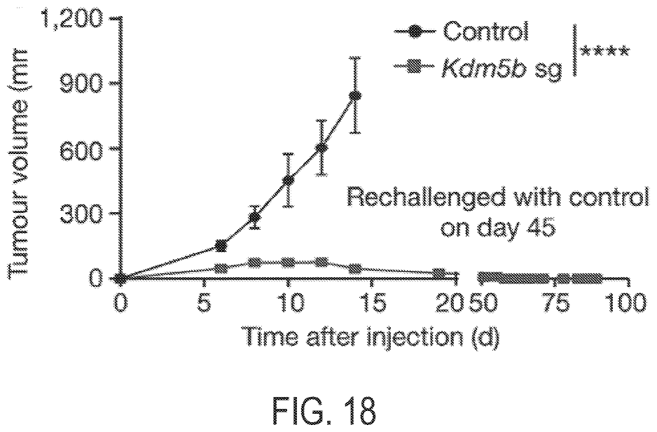

Preliminary data shows that late treatment with a small molecule inhibitor of KDM5B recovers effector and memory programs in dysfunctional human CD8 T cells, as well as improving their cytotoxic activity in vitro. FIGS. 12A-12F. Selective KDM5B inhibitor treatment shifts differentiation of Tprog cells to the cytolytic, rather than terminally exhausted state, thereby enhancing T cell responses to ICB therapy. FIGS. 15A-15C; FIG. 18; FIGS. 16A-16C; FIG. 17. Therapeutic benefits of a selective KDM5B inhibitor were tested using preclinical models of T cell exhaustion, as well as adoptive transfer experiments of Tprog cells to determine whether targeting KDM5B: skews Tprog cell differentiation towards the cytolytic state by impeding terminal exhaustion-associated DNA methylation; and 2b) synergizes with ICB therapy to enable durable TEX responses.

Discussed herein are the mechanisms by which epigenetic modifications regulate terminal T cell-exhaustion and how this epigenetic interplay can be targeted to improve the efficacy of ICB therapy.

### Research Strategy

The selective killing power of CD8 T cells can be exploited by either rejuvenating exhausted cells with ICB therapy, or by re-directing their specificities with chimeric antigen receptors (CAR) to attack cancer cells expressing tumor antigens. Still, terminal T cell-exhaustion remained a major obstacle to these immunotherapies. (McLane (2019); Sharma (2020); Long (2015)) During persistent TCR (T cell receptor) stimulation, cytotoxic T cells progressively lose their effector functions, transitioning through a less-exhausted state, including the “stem-like” or “progenitor” and “cytolytic” subsets that remain ICB-responsive, before they become terminally exhausted and refractory to ICB therapy. (McLane (2019)) This progression to terminal exhaustion may explain why many patients relapse and fail to mount durable ICB responses. In addition, the therapeutic efficacy and in-vivo persistence of CAR T cells is limited, in part, due to their progression toward exhaustion. (Long (2015); Yang (2017); Deng (2020)) Thus, a better understanding of the molecular mechanisms underlying terminal T cell-exhaustion boosts T cell immunotherapies.

Epigenetic programming is a fundamental mechanism that regulates gene expression and cell fate commitment. (Bintu (2016); Bird (2002)) We and other groups have shown that TEX cells acquire distinct epigenetic programs that lock them in a dysfunctional state. (McLane (2019); Yousif (2021); Saadey (2022); Ghoneim (2016)) These epigenetic maps provide a mechanistic basis for the maintenance of gene expression programs in TEX cells. Discussed herein are how the epigenetic changes initiated and orchestrated in TEX cell subsets whether epigenetic programming in Tterm cells can be blocked or reversed to a functional state. Bridging these gaps allows for identification and targeting of factors that apply “epigenetic brakes” to CD8 T cell function.

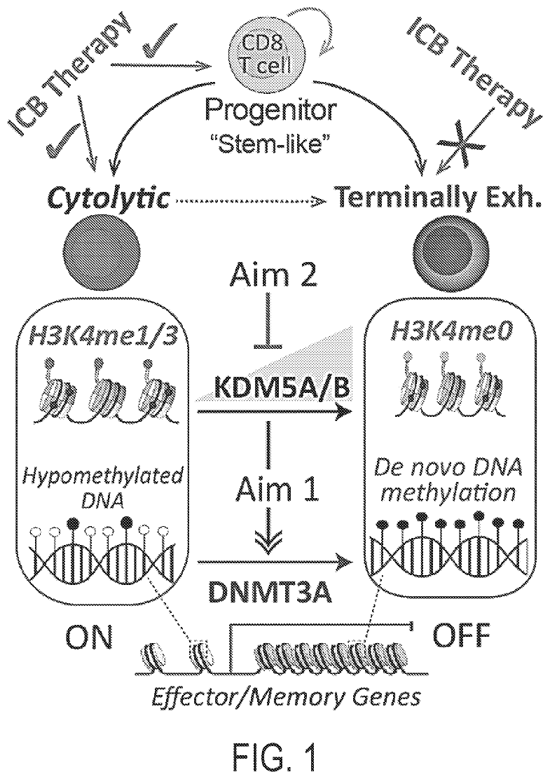

Exhaustion-associated epigenetic scarring has been a major barrier to rejuvenation efforts of TEX cells for treating chronic viral infections or cancer. De novo DNA methyltransferase 3a (Dnmt3a) enforces terminal T cell-exhaustion by epigenetically silencing key genes that regulate T cell functions. (Ghoneim (2017)) The efficacy of ICB therapy was enhanced through targeted inhibition of T cell-intrinsic de novo DNA methylation. (Ghoneim (2017)) Genetic deletion of Dnmt3a in activated CD8 T cells or therapeutic erasure of DNA methylation programs by decitabine treatment synergizes with ICB therapy to rescue the response of TEX cells. Yet, decitabine is a non-selective, highly cytotoxic drug that has adverse effects on other immune cells. One goal herein was to identify key factors regulating de novo epigenetic programs that drive CD8 T cells to terminal exhaustion, and to target these factors for enhancing T cell immunotherapies. Instead, the data revealed a novel epigenetic regulator of T cell exhaustion and therefore teach an innovative approach to enhance ICB response by blocking progression towards terminal exhaustion (FIG. 1). Discussed herein is the investigation of the role of this regulator in promoting exhaustion-associated de novo epigenetic changes, which will provide insights into novel strategies to enhance the efficacy of T cell immunotherapies by epigenetic reprogramming.

### KDM5B-Mediated Loss of H3K4Me3 Modifications Primes De Novo DNA Methylation Programming in Terminally-Exhausted CD8 T Cells.

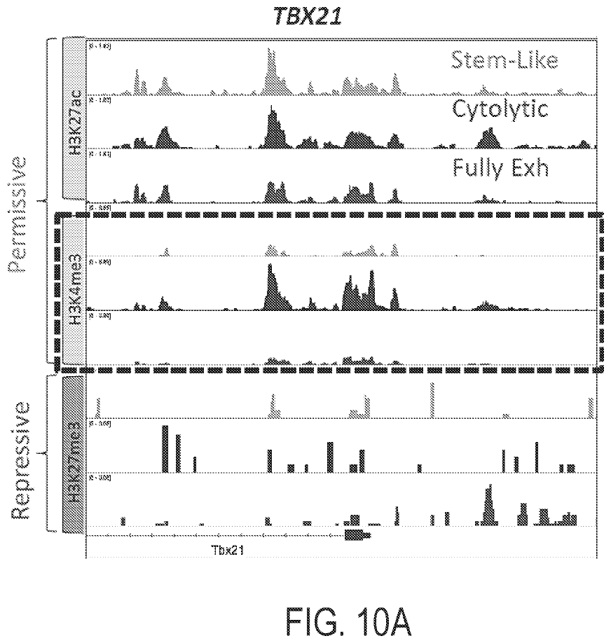

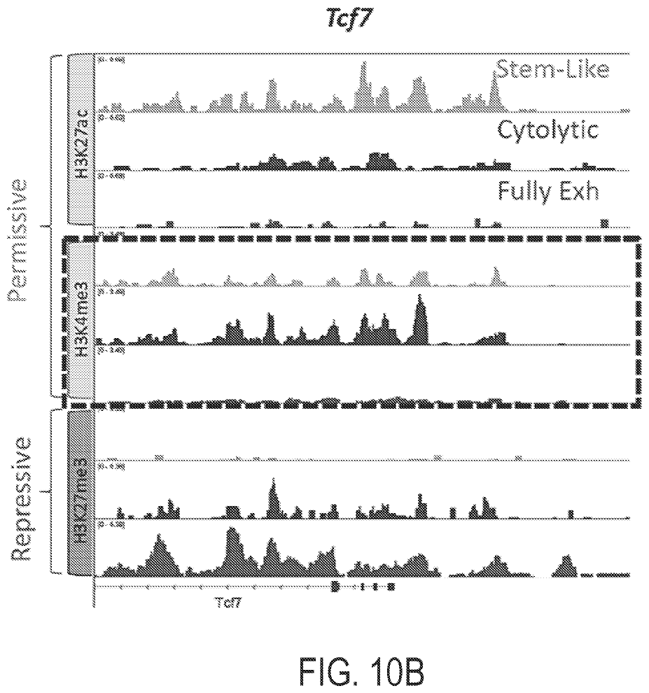

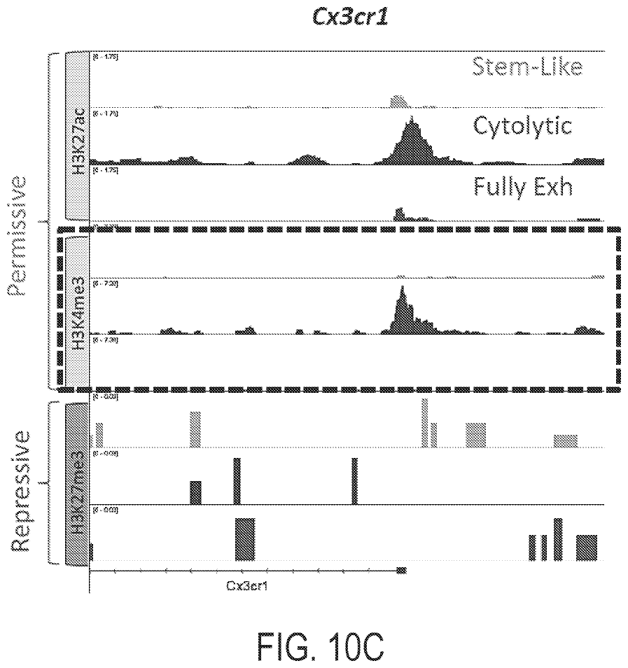

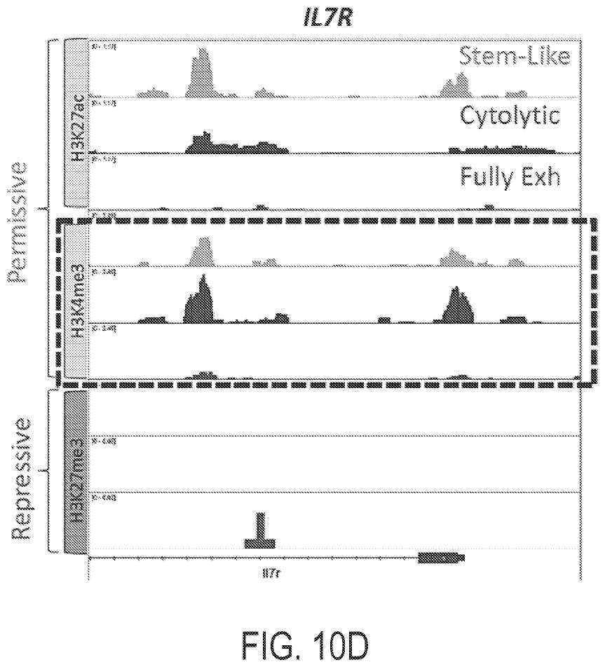

Dnmt3a-mediated epigenetic programs enforce terminal exhaustion of chronically stimulated CD8 T cells and restrain their response to ICB. Previous data reveal an important role of the permissive H3K4me3 marks in inhibiting the Dnmt3a activity, a significant loss of H3K4me3 modifications in Tterm cells and a remarkable maintenance of H3K4m3 marks at effector/memory genes in Tcyto cells. FIGS. 10A-10D. These findings imply an intricate regulatory crosstalk between H3K4me3 modifications and de novo DNA methylation, that ensures silencing of both function and reprogramming capacity of CD8 T cells as they progress towards terminal exhaustion. This data support indicate that loss of H3K4me3 marks enables de novo DNA methylation programming that skews CD8 T cell differentiation towards terminal exhaustion and offsets the generation of the cytolytic subset.

### Blocking Epigenetic Programming of Exhausted T Cells Will Enhance the Efficacy and Durability of T Cell-Immunotherapies.

Further data indicate that: a) inhibiting de novo DNA methylation synergizes ICB responses and promotes durable rejuvenation after stopping ICB treatment; b) increased KDM5B expression is coupled with terminal dysfunction; while c) therapeutic KDM5B inhibition rescues effector function in chronically stimulated human CD8 T cells. Based on these findings, targeting KDM5B activity prevents Dnmt3a-mediated silencing of functional programs in Tterm cells. Thus, the selective KDM5B inhibitor treatment provides an innovative therapeutic strategy to hinder de novo DNA methylation programming in TEX cells, rescuing their responses and promoting long-lived immunity following ICB therapy.

### Novel In-Vitro Model of Terminal T Cell-Dysfunction to Study Epigenetic (Re)Programming in Persistently Stimulated Human CD8 T Cells.

The in vitro model system establishes a stable dysfunctional program that contrasts with other recently reported in vitro CAR T cell systems. These models produced only transient dysfunction of CAR T cells, which was reversible upon resting. (Good (2021); Weber (2021)) Thus, the tractable model facilitate mechanistic investigations of human terminal T cell-dysfunction and guide complementary in vivo animal studies. State-of-the-art multi-omics were used to profile both global DNA methylation and histone H3K4 methylation in small numbers of CD8 T cell subsets, revealing the spectrum of epigenetic programs within TEX cell subsets. This approach provides insights into CD8 T cell functional plasticity and responsiveness to ICB therapy.

### Approach

Terminal exhaustion is a challenge to T cell immunotherapies. Therefore, it is helpful to identify cell-intrinsic barriers for rejuvenation of Tterm cells during chronic infections or cancer. Disclosed herein is an approach to block or reverse progression towards the terminally-exhausted state by targeting epigenetic modifiers.

### De Novo DNA Methylation is Essential for Establishing Terminal T Cell Exhaustion

Dnmt3a-mediated de novo DNA methylation plays a role in establishing T cell exhaustion. Conditional deletion of Dnmt3a (Dnmt3a cKO) in activated CD8 T cells blocks their progression towards terminal exhaustion. Furthermore, Dnmt3a-deficient antigen-specific CD8 T cells are exhaustion-resistant during lifelong chronic lymphocytic choriomeningitis virus (LCMV) infection—an animal model of severe T cell exhaustion. At the mechanistic level, virus-specific cKO CD8 T cells were maintained at higher numbers in lymphoid and non-lymphoid tissues, retained high capacity to recall effector cytokines (e.g., IFNg, IL-2, TNF), and continued to express higher levels of stemness-associated transcription factors, such as Tcf1 and Lef1, relative to the wild type (WT) CD8 T cells, despite prolonged TCR stimulation and high PD-1 expression. (Ghoneim (2017)) The data established de novo DNA methylation as a novel, cell-intrinsic mechanism that is causally linked to T cell exhaustion.

### De Novo DNA Methylation Enforces Silencing of Effector and Stemness Programs in TEX Cells Through Remodeling the Open Chromatin Landscape.

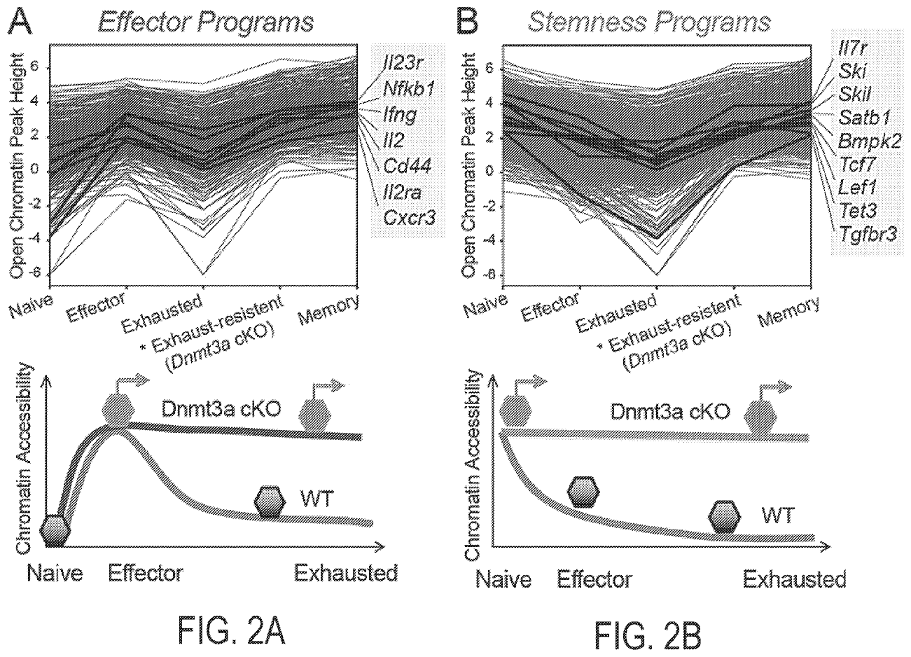

Recent studies have shown that TEX cells undergo distinct changes in global chromatin accessibility and histone modifications during chronic viral infections or cancer. (Philip (2017); Pauken (2016); Scott-Browne (2016); Sen (2016); Daniel (2022); Giles (2022)) Furthermore, exhaustion-specific open chromatin landscape can be maintained in the absence of antigen, suggesting an underlying cell-intrinsic mechanism. (Yousif (2021); Angelosano (2012); Utzschneider (2013)) To better understand the heritable nature of exhaustion-specific programs, whole-genome DNA methylation changes were profiled in antigen-specific WT and Dnmt3a-cKO CD8 T cells during acute and chronic LCMV infections on day 35 post-infection (p.i). WT cells progressively acquired >2000 newly methylated regions during naïve-to-effector-to-exhausted transitions. Many of these differentially methylated regions (DMRs) were acquired in genes that regulate T cell function, metabolic activity and self-renewal, such as Ifng, Tbx21 (encoding T-bet), Myc, and Tcf7 (encoding Tcf1). (Ghoneim (2017) Chromatin accessibility changes were profiled in antigen-specific WT and Dnmt3a cKO CD8 T cells during chronic LCMV infection to determine whether Dnmt3a-mediated epigenetic programming regulates the open chromatin landscape of TEX cells. DNA methylation and chromatin accessibility changes are inversely linked, with >60% of exhaustion-associated open chromatin regions (OCRs) regulated by Dnmt3a. The majority of Dnmt3a-regulated OCRs are acquired at the post-effector stage. cKO CD8 T cells retain chromatin openness at many effector function and stemness-related genes relative to WT exhausted cells (FIG. 2). The findings indicate that de novo DNA methylation serves as a T cell-intrinsic mechanism that, in part, shapes the open chromatin landscape enforcing terminal exhaustion. The data herein causally link de novo DNA methylation to terminal exhaustion in T cells, and that blocking T cell-intrinsic DNA methylation augments responses to ICB therapy.

### Define the Role of H3K4Me3 Modifications in Regulating Exhaustion-Associated De Novo DNA Methylation Programs.

The data show that Dnmt3a-mediated DNA methylation plays a crucial role in establishing T cell-exhaustion. H3K4me3 and H3K4me1 are chromatin hallmarks of active promoters and enhancers, respectively. Importantly, biochemical and structural studies indicate that unmethylated H3K4 residues activate the DNMT3A enzyme. (Guo (2015); Li (2011); Weber (2007); Zhang (2010)) Discussed herein are the role of H3K4me3 modifications in regulating the initiation of de novo DNA methylation in TEX cells as they differentiate towards a terminally exhausted state.

Discussed herein is KDM5B-mediated loss of H3K4me3 marks at effector/memory genes enables de novo DNA methylation programming that enforces terminal T cell-exhaustion. This is based on in vitro and in vivo models of exhaustion revealed that terminally dysfunctional human and mouse CD8 T cells: (1) express high levels of KDM5B; (2) undergo H3K4me3 demethylation, coupled with de novo DNA methylation that enforces reduced chromatin accessibility and transcriptional activity of effector and memory genes; and (3) maintenance of H3K4me3 marks inhibits DNMT3A activity. (Guo (2015); Li (2011); Weber (2007))

### Terminally Exhausted T Cells Lose H3K4Me3 Marks and Undergo De Novo DNA Methylation at Effector Memory-Related Loci.

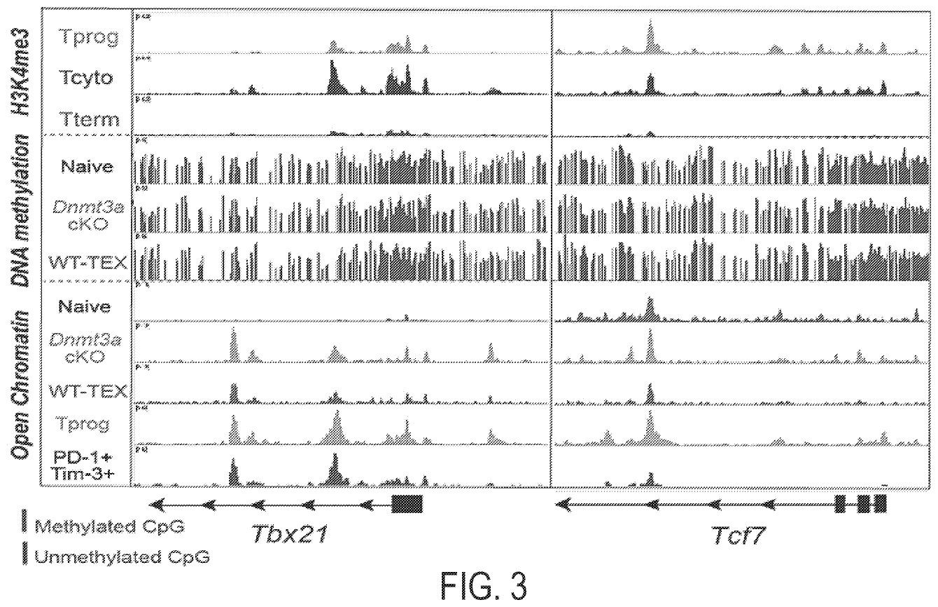

To determine whether changes in histone H3K4me3 modifications correlate with de novo DNA methylation and subsequent chromatin accessibility changes in TEX cell subsets, H3K4me3 profiles of LCMV-specific TEX subsets were integrated with the DNA methylation datasets and unpublished open chromatin landscapes of naïve and chronically stimulated Dnmt3a-cKO or WT LCMV-specific CD8 T cells, as well as chromatin accessibility profiles of LCMV-specific Tprog cells or Cxcr5− Tim-3+CD8 T cells isolated from chronically LCMV-infected animals (FIG. 3). Tterm cells lose H3K4me3 modifications at effector and memory-related genes, such as Tbx21, Tcf7, Lef1, Ifng, and Myc. These changes in H3K4me3 modifications were coupled with de novo DNA methylation programs and reduced chromatin accessibility in WT TEX cells (examples are shown in FIG. 3). In contrast, chronically stimulated Dnmt3a-deficient cells retained chromatin openness at these loci-patterns similar to those in the Tprog and Tcyto subsets. (Chen (2021)) This data indicate that, during their progression towards terminal exhaustion, CD8 T cells lose H3K4me3 marks and undergo de novo DNA methylation that enforces chromatin inaccessibility and gene silencing.

### Cytolytic CD8 T Cells Downregulate KDM5B and Maintain High H3K4Me3 Levels at Effector Genes.

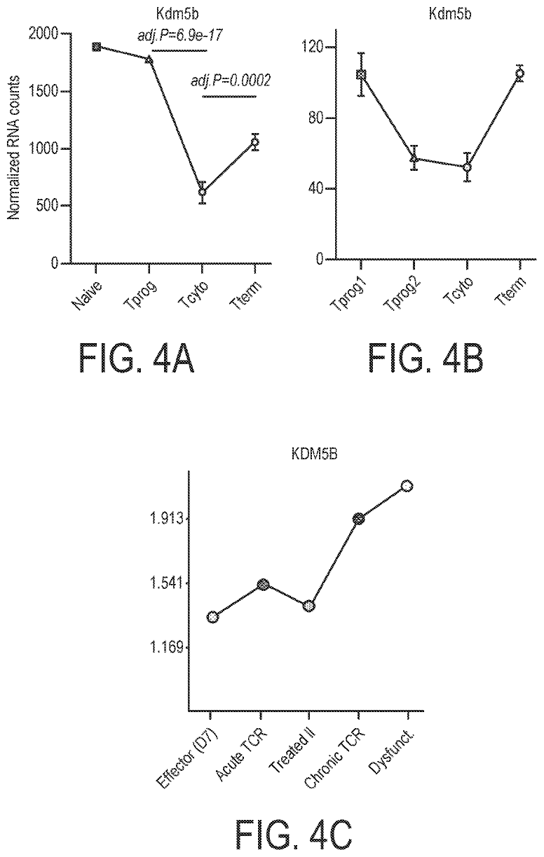

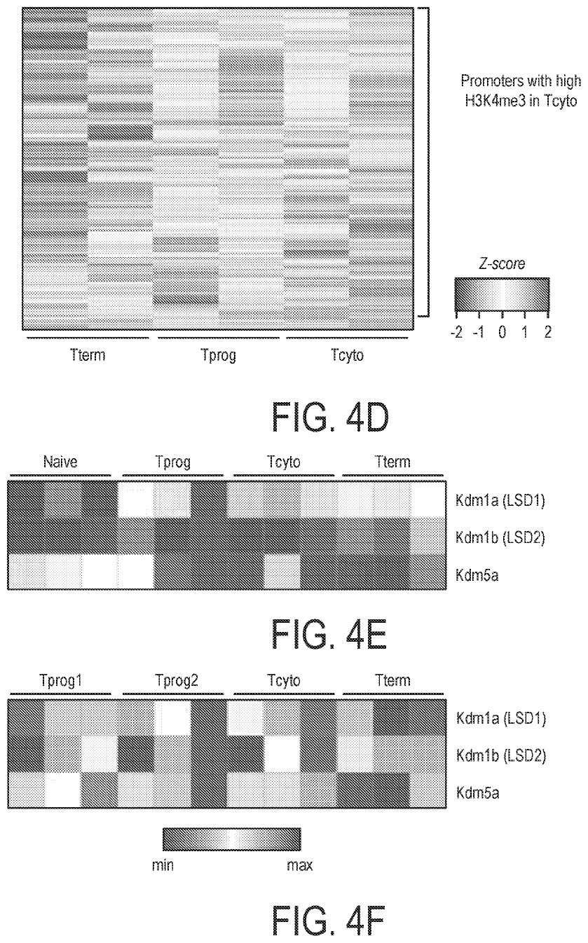

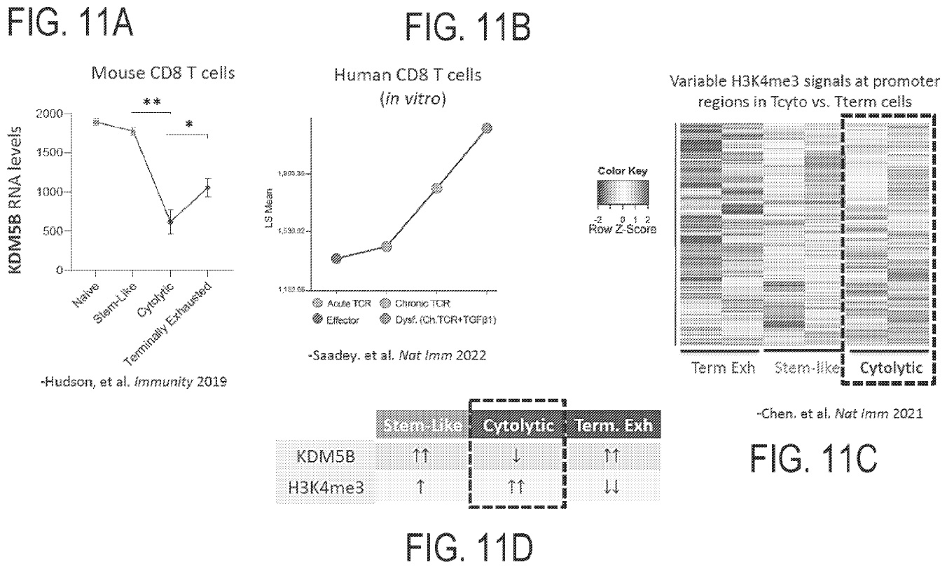

To better understand factors that affect H3K4me3 changes in TEX cells, expression levels of key H3K4 demethylases were examined in TEX cell subsets. The Tprog and Tterm subsets have a high expression of Kdm5b, while Tcyto cells and the intermediate subset “Tprog2” downregulate Kdm5b (FIG. 4A,B). Additionally, polyfunctional human CD8 T cells have lower levels of KDM5B compared to dysfunctional CD8 T cells that develop under chronic TCR plus TGF(31 stimulation conditions (FIG. 4C). (Saadey (2022)) The downregulation of Kdm5b can be linked to the maintenance of H3K4me3 marks in the Tcyto subset (FIG. 4D). Murine Tterm cells, but not dysfunctional human CD8 T cells, have increased levels of Kdm5a, which only demethylates H3K4me3/me2 marks (FIG. 4E,F). (Black (2012)) In contrast, Kdm5b enzyme can demethylate tri-, di-, and mono-methylated H3K4 residues resulting in unmethylated H3K4 tails that activate DNMT3A enzyme. (Guo (2015); Li (2011); Zhang (2010)). FIGS. 11A-11D.

Determining Whether De Novo DNA Methylation Tracks with an Initial Loss of H3K4Me3 in Tterm Cells.

The integrating epigenetic analysis demonstrate that Tterm cells undergo H3K4 demethylation at effector and memory-related genes that also gain DNA methylation programs and lose chromatin accessibility within WT TEX cells. To rigorously define the temporal role of H3K4me3 changes in regulating the progressive acquisition of DNA methylation programs during the development of terminal exhaustion, H3K4me3 ChIP-sequencing and whole-genome bisulfite sequencing (WGBS) assays of virus-specific effector CD8 T cells and TEX subsets were performed (Table 1), isolated from spleens of chronically LCMV-infected animals on days 8 and post-infection (p.i), respectively. In addition, H3K4me3 changes in acutely stimulated or chronically stimulated dysfunctional human CD8 T cells were profiled. These epigenetic profiles reveal the spectrum of changes in H3K4me3 and DNA methylation programs within effector CD8 T cells as they develop terminal dysfunction. These data pinpoint the temporal impact of H3K4me3 changes on CD8 T cell's de novo DNA methylation programming that drives and enforces a terminal dysfunction fate.

The integrative epigenetic analyses reveals an inverse relationship between H3K4me3 and DNA methylation programs at effector and memory-related loci within mouse and human CD8 T cells as they develop terminal dysfunction. It is possible that Tprog cells undergo H3K4 demethylation at genes that later acquire DNA methylation events during cell proliferation and differentiation to terminal exhaustion. To dissect these epigenetic events, Tprog cells were alternatively isolated from chronically LCMV-infected animals on day 35 p.i and adoptively transferred into congenially distinct, infection-matched animals. After 10-14 days, H3K4me3 and DNA methylation changes were isolated and profiled in the adoptively transferred cells that develop a Tterm phenotype under chronic stimulation settings. Tracking epigenetic changes pre- and post-adoptive transfer reveals the kinetics of H3K4me3 loss as Tprog cells acquire DNA methylation programs that enforce terminal exhaustion. depleted chronic LCMV infection model were used to ensure the lifelong persistence of viral antigens.

### To Determine Whether Downregulating KDM5B in Chronically Stimulated CD8 T Cells Preserves H3K4Me3 Marks and Hinders Exhaustion-Associated DNA Methylation.

The highly cytolytic subset of TEX cells express lower levels of KDM5B relative to Tterm cells, suggesting a negative impact of KDM5B on CD8 T cell functions. To determine whether KDM5B activity is essential for the initiation of exhaustion-associated DNA methylation programming, KDM5B was knocked down in ex vivo-stimulated, cord blood-derived human CD8 T cells using our optimized shRNA-retroviral vector (RV) transduction protocol (day 7). Then, the genetically modified or control CD8 T cells were maintained under chronic TCR plus TGF(31 stimulation conditions that establish terminal dysfunction. (Saadey (2022)) On day 28, WGBS, H3K4me3 ChIP-seq, and RNA-seq, and measure effector functions and cytolytic activity of KD and control human CD8 T cells were performed. In addition, the in vitro model of mouse T cell exhaustion using TCR-transgenic Thy1.1+P14 CD8 T cells was used (Saadey (2022)), and knockdown Kdm5b in ex vivo-stimulated P14 cells (day 6), followed by adoptive transfer of KD or control P14 cells into chronically LCMV-infected C57BL/6 animals on day 8 p.i. On day 28, P14 cells were isolated and measure their phenotypic and functional changes and profile their DNA methylation epigenomes. Comparing DNA methylation changes in chronically stimulated KD versus control CD8 T cells reveals the impact of modulating H3K4me3 levels on exhaustion-specific DNA methylation programs.

We anticipate that downregulating KDM5B will preserve H3K4me3/me1 marks in mouse and human CD8 T cells which will prevent the acquisition of de novo DNA methylation, thereby curbing their progression to a terminal dysfunction state at the functional, transcriptional and epigenetic levels. It is possible that the RV-transduction process decreases the survival of human CD8 T cell under in vitro chronic stimulation settings. As an alternative approach, we will inhibit KDM5B activity in human CD8 T cells using a selective KDM5B inhibitor on days 14-28, followed by integrated functional and epigenetic analyses.

TABLE 1
Surface markers to identify and sort TEX cell subsets.
Subsets of polyclonal exhausted CD8 T cells (CD8+ CD44+ PD-1+):

| | |
|---|---|
| I) Tprog cells | Ly108+ Cx3cr1− Tim-3− |
| II) Tcyto cells | Ly108− Cx3cr1+ Tim-3−/+ |
| III) Tterm cells | Ly108− Cx3cr1− Tim-3+ |

Determine Whether Targeting KDM5B in TEX Cells Enhances their ICB Responses by Curbing Epigenetic Progression Towards Terminal Exhaustion.

The data indicate that blocking de novo DNA methylation in CD8 T cells renders them exhaustion-resistant and promotes durable responses to ICB therapy. Tested herein is the therapeutic efficacy of targeting KDM5B activity in TEX cells for enhancing ICB responses and investigated herein is the molecular mechanism underlying this new therapeutic strategy.

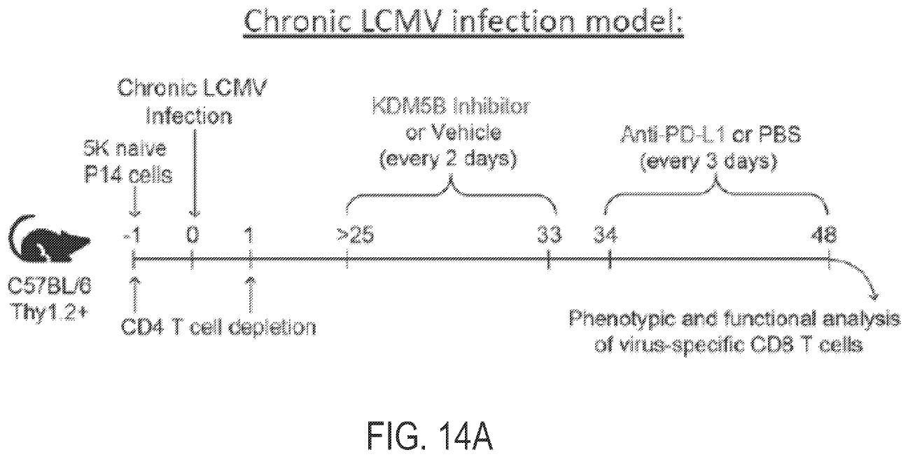

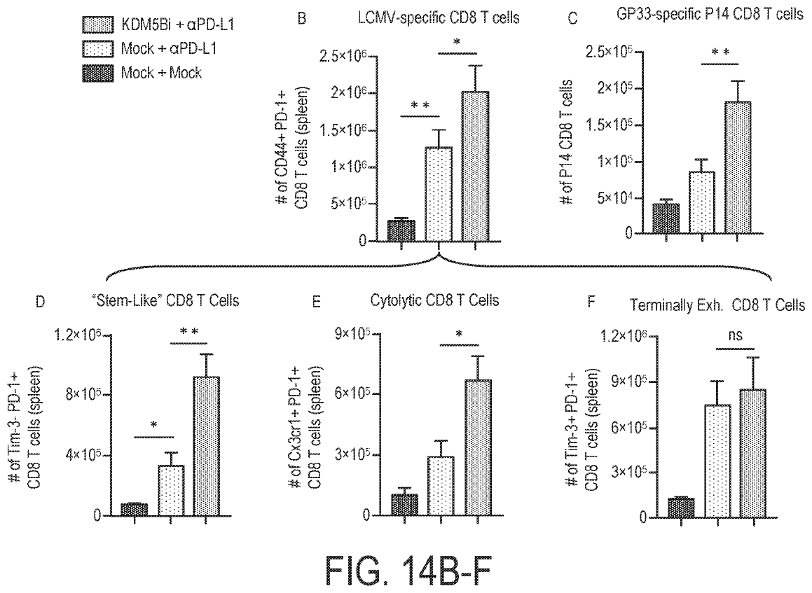

Discussed herein is whether therapeutic targeting of KDM5B in chronically stimulated CD8 T cells synergizes their ICB responses by impeding epigenetic progression of Tprog cells towards terminal exhaustion. This discussion is based on three observations: (1) Blocking de novo DNA methylation in persistently stimulated CD8 T cells skews their open chromatin landscape toward a functional signature and preserves their rejuvenation responses after ICB is discontinued. (2) Therapeutic KDM5B blockade reactivates effector and memory programs in dysfunctional human T cells in vitro, and (3) enhances TEX cell responses to ICB therapy in vivo. FIGS. 14A-14F.

Blocking De Novo DNA Methylation in CD8 T Cells Preserves their Potential for Rejuvenation after Cessation of ICB.

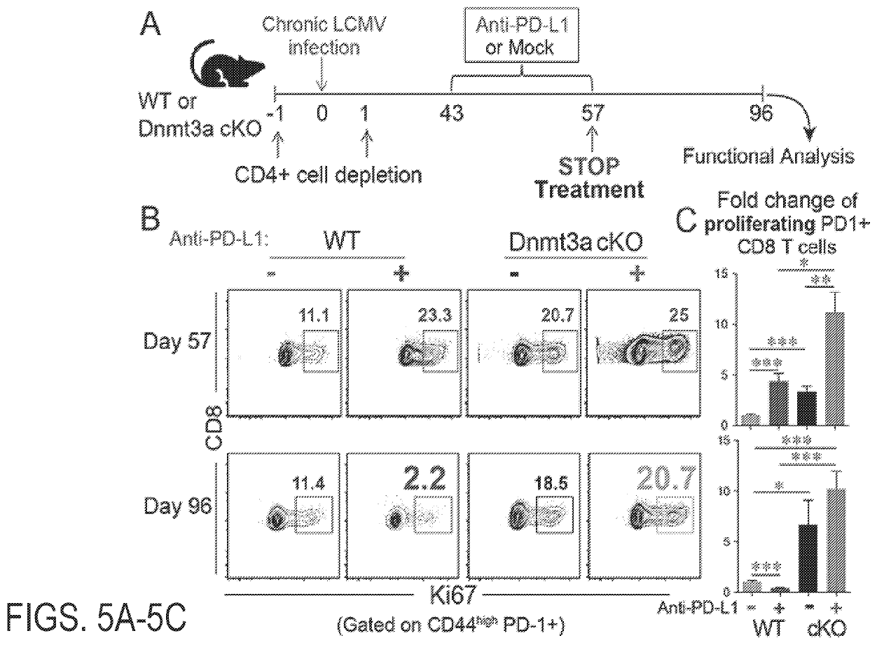

Many patients fail to develop durable protection against tumor relapses after ICB treatment is discontinued, further establishing the need to develop complementary therapeutic strategies for generating durable rejuvenation of TEX cells. Based on the data herein, de novo DNA methylation in TEX cells substantially prolongs their proliferation activity after discontinuation of ICB treatment. In contrast to WT CD8 T cells, Dnmt3a-deficient cells were maintained at significantly higher numbers during lifelong chronic LCMV infection and exhibited high proliferative capacity for >1 month after cessation of ICB treatment (FIG. 5). These unpublished findings indicate that blocking epigenetic programs within TEX cells may provide an effective strategy to preserve the proliferative response and memory potential after ICB therapy.

Therapeutic Targeting of KDM5B Recovers Effector and Memory Programs in Dysfunctional CD8 T Cells and Enhances their ICB Responses.

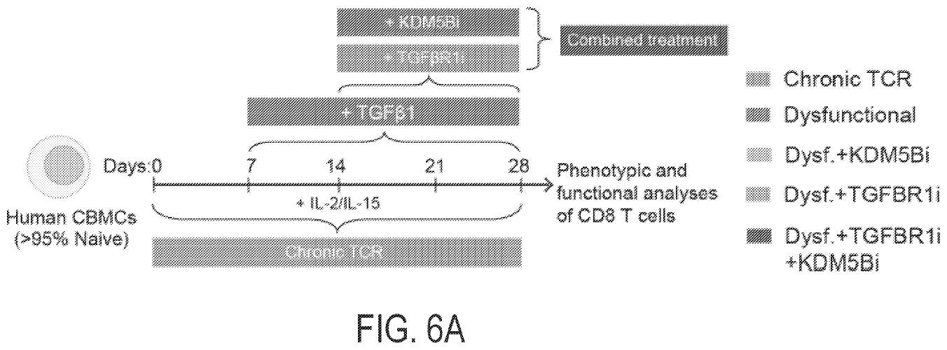

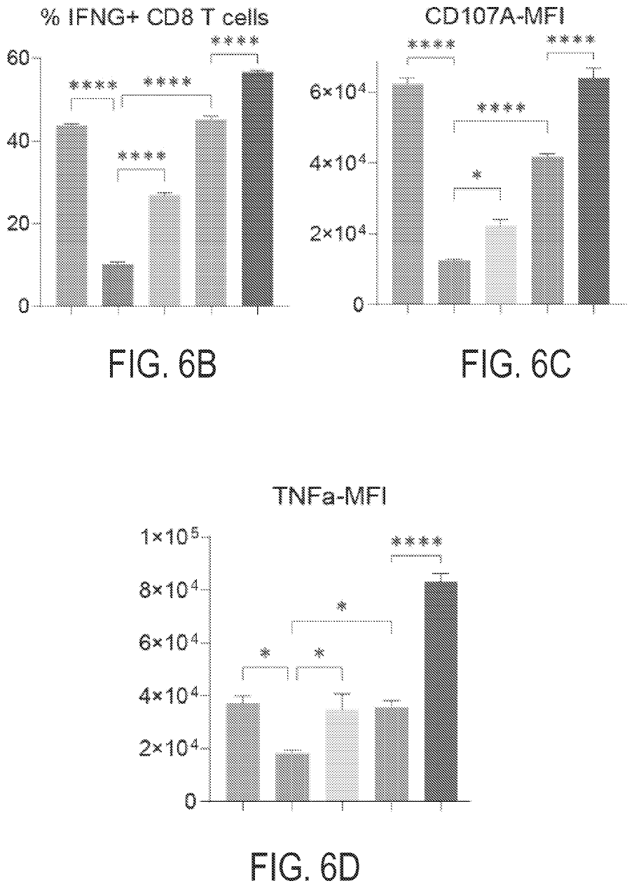

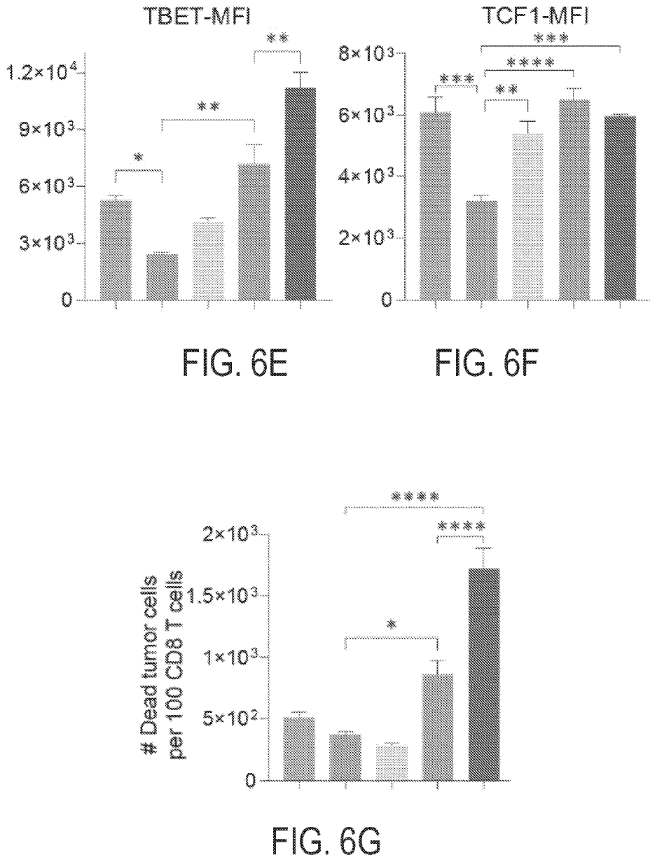

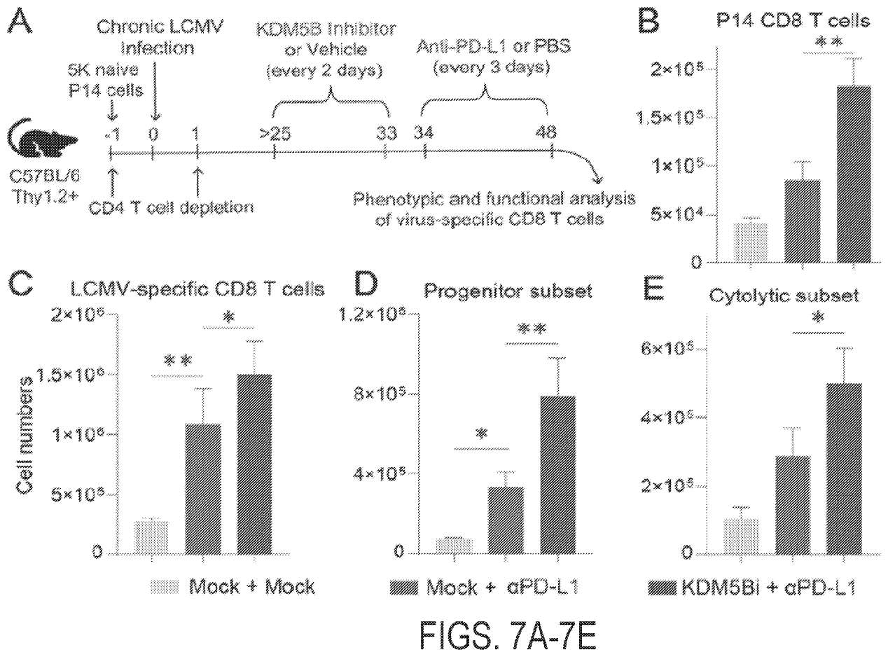

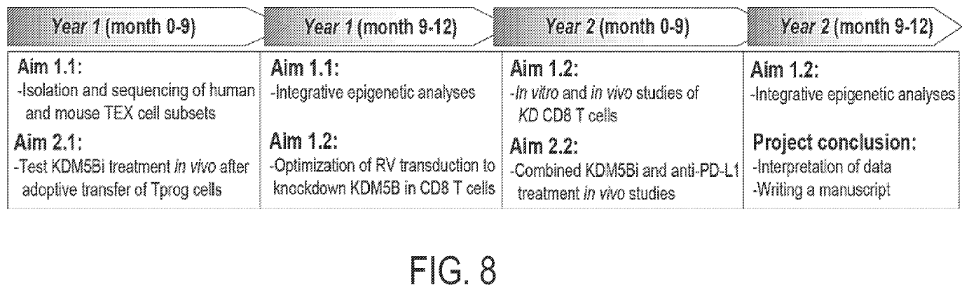

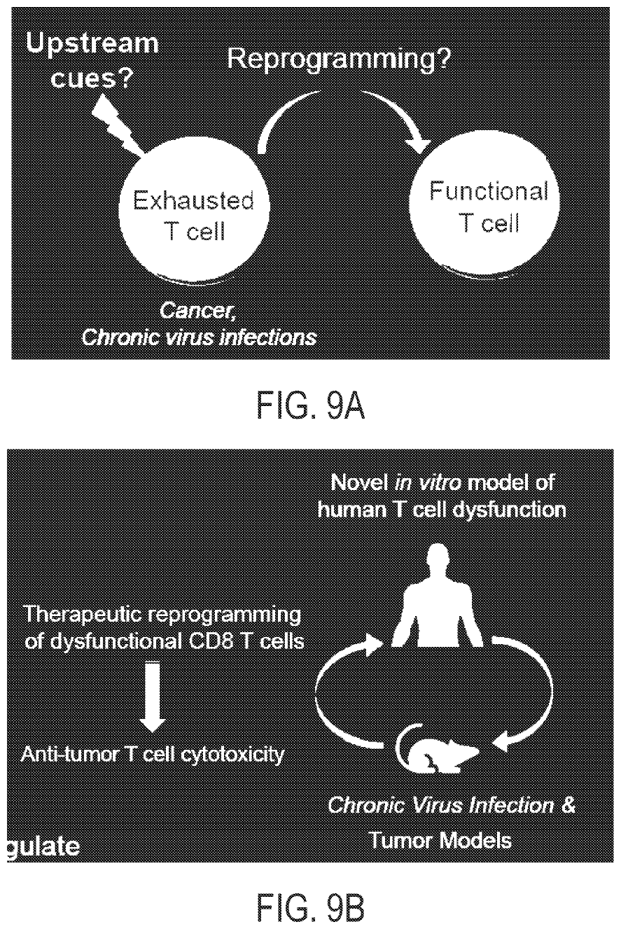

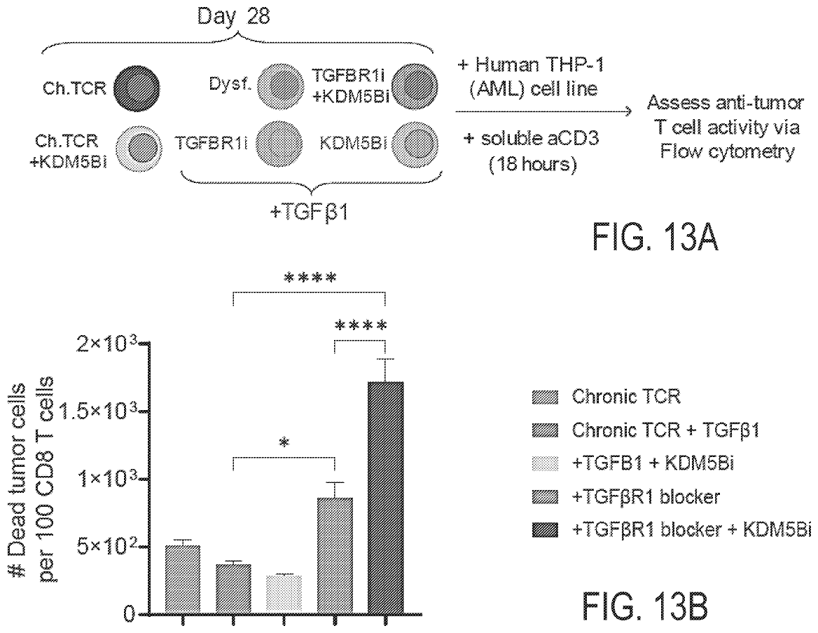

To test the therapeutic efficacy of targeting KDM5B in dysfunctional CD8 T cells, chronically TCR plus TGFβ1-stimulated human T cells were treated with a selective inhibitor of KDM5B (Johansson (2016)) on day 14-28 with or without TGFBR1 blocker. While TGFβ1 blockade recovered effector functions of chronically stimulated CD8 T cells, KDM5Bi treatment further potentiated the reactivation of polyfunctionality and memory expression programs, and augmented tumor-killing activity (FIG. 6). KDM5Bi monotherapy significantly restored effector cytokine secretion and expression of memory-related transcription factors in dysfunctional human CD8 T cells (FIG. 6, FIGS. 13A-13B). The efficacy of KDM5Bi therapy was also tested during lifelong chronic LCMV infection in vivo. Late KDM5Bi treatment (days 25-33 p.i) enhanced the responses of virus-specific TEX cells to PD-L1 blockade, with significant expansion of both Tprog and Tcyto cells (FIG. 7). These findings demonstrate the therapeutic efficacy of KDM5B blockade to enhance the rejuvenation of TEX cells.

### To Determine Whether Targeting KDM5B Skews Tprog Cell Differentiation Towards the Cytolytic State by Impeding Exhaustion-Associated DNA Methylation.

Data showed that KDM5B inhibition can potentiate the recovery of effector functions of chronically stimulated human CD8 T cells in vitro. Here, it was tested if a selective KDM5Bi treatment can enhance CD8 T cell responses by blocking their epigenetic progression towards terminal exhaustion in vivo using CD4-depleted chronic LCMV infection model. On day 30 p.i, virus-specific Thy1.1+Tprog cells were isolated, followed by CTV-labeling and adoptive transfer into infection-matched C57BL/6 animals. KDM5Bi or vehicle treatment then started on days 31-41 p.i. Following treatment regimen, the quantity, phenotype, proliferative potential, and functional capacity was measured of adoptively transferred CD8 T cells using flow cytometry. In addition, Oxford Nanopore-seq technology was used to measure DNA methylation levels at exhaustion-specific DMRs in the transferred CD8 T cells, following a protocol that was previously optimized for small cell numbers in the laboratory. (Saadey (2022)) The data reveal the effects of therapeutic KDM5B blockade on the Tprog-to-Tterm differentiation at the functional and epigenetic levels.

The KDM5Bi treatment increased the quantity of Tcyto cells while impeding the development of Tterm cells by decreasing the acquisition of de novo DNA methylation events at effector and memory genes. However, KDM5Bi therapeutic effects were mediated by modulating CD4 T cell differentiation, such as regulatory or follicular helper CD4 T cells. Therefore, Tprog cells were alternatively transferred into infection-matched Ragi-KO mice which lack T and B cells, followed by KDM5Bi therapy. This approach determined whether the therapeutic benefits of KDM5B inhibition are CD4 T cell-independent.

To Test Whether Selective KDM5Bi Treatment Synergizes with ICB Therapy to Generate Durable TEX Responses.

The data indicate that blocking de novo DNA methylation programming in TEX cells promotes their durable responses to ICB therapy. Discussed herein is testing the durability of the ICB responses in the treatment-modulated P14 CD8 T cells under both chronic LCMV infection and gp33-expressing C2TA tumor settings. After blocking Kdm5b activity in the transferred CD8 T cells by KDM5Bi therapy (days 22-32 p.i; days 16-26 post-tumor implantation), chimeric mice were treated with anti-PD-L1 for 2 weeks. changes in phenotype, function, and proliferation response of P14 cells were then tracked over 1 month after cessation of PD-L1 blockade. The memory potential of the rejuvenated CD8 T cells was tested by transferring CTV-labeled, control or KDM5Bi-treated P14 cells after stopping ICB into LCMV-immune or tumor-naive mice. Then, their self-renewal capacity was assessed by tracking of the CTV dilution of the transferred T cells. Their recall effector function was tested by challenging the recipient mice with the same tumor cells or a high dose of LCMV clone 13.

KDM5Bi treatment-induced reprogramming of exhausted P14 cells synergized their proliferation response and promote long-lived memory function after ICB treatment. Short-term KDM5Bi treatment may not induce permanent epigenetic reprogramming of TEX cells KDM5B blockade may extend during anti-PD-L1 treatment to protect the rejuvenated CD8 T cells from converting to the terminal-exhaustion state under settings of persistent antigen exposure.

There is also a possibility that some C2TA tumors continue growing after stopping PD-L1 blockade. Alternatively, the rejuvenated CD8 T cells were isolated from tumor-bearing mice and their memory tested and effector function recalled by transferring them into LCMV-immune or naive mice as described above. Discussed herein is the therapeutic significance of targeting KDM5B enzyme in TEX cells. Further provided are insights into designing novel approaches to block terminal exhaustion and enhance the efficacy of T cell-based immunotherapies.

### Example 2: H3K4 Demethylases KDM5A/B Regulating De Novo DNA Methylation Programs to Mitigate Terminal Exhaustion

### Introduction

Cytotoxic CD8 T lymphocytes provide adaptive defense against tumors or virus-infected cells. However, under prolonged antigen exposure, CD8 T cells lose their killing function and become exhausted, failing to eliminate cancer or chronic viral infections. T cell exhaustion occurs progressively, transitioning through three major subsets: (a) progenitor (Tprog) cells that act as a chronic source for other subsets, (b) a highly cytolytic subset (Tcyto), and (c) terminally exhausted (Tterm) cells that lose the effector functions. Immune checkpoint blockade (ICB) is a revolutionary cancer immunotherapy that reinvigorates the Tprog and Tcyto subsets by blocking signals from inhibitory receptors (e.g., CTLA-4, PD-1). Yet, Tterm cells remain refractory to ICB, perhaps explaining why many patients remain nonresponsive whose anti-tumor T cells are terminally exhausted. The epigenome of Tterm cells plays a role in locking their fate commitment.

As such, the long-term goal is to understand how terminal exhaustion is acquired in T cells, and to block or reverse this process to boost T cell immunotherapies.

De novo DNA methylation, mediated by the Dnmt3a enzyme, silences T cell function and enforces terminal exhaustion, thereby restraining the efficacy of ICB therapy. However, key regulators of de novo epigenetic programs that govern T cell dysfunction remain largely unknown.

Disclosed herein is harnessing the crosstalk among epigenetic mechanisms in CD8 T cells. A novel in vitro model that establishes an epigenetically stabilized, terminal dysfunction in human CD8 T cells was used to investigate these mechanisms. The data revealed an role for the activating H3K4me3 marks in regulating CD8 T cell (dys)function. Loss of H3K4me1/3 marks at effector/memory genes enables de novo DNA methylation programming that enforces terminal exhaustion of CD8 T cells. However, therapeutic targeting of the H3K4 demethylases, KDM5A/B, in CD8 T cells blocks their progression towards terminal exhaustion, enhancing the ICB response.

Discussed herein is the casual link between de novo DNA methylation and terminal exhaustion in T cells, and that blocking T cell-intrinsic DNA methylation augments responses to ICB therapy.

Further, discussed herein is whether KDM5A/B-mediated loss of H3K4me3 marks at effector/memory genes enables de novo DNA methylation programming that enforces terminal T cell-exhaustion. This is based on three observations—The in vitro and in vivo models of exhaustion revealed that terminally dysfunctional human and mouse CD8 T cells: (1) express high levels of KDM5A/B; and (2) undergo H3K4me3 demethylation, coupled with de novo DNA methylation that enforces reduced chromatin accessibility and transcriptional activity of effector and memory genes. (3) Maintenance of H3K4me3 marks inhibits DNMT3A activity. Discussed herein is whether therapeutic targeting of KDM5A/B in chronically stimulated CD8 T cells synergizes their ICB responses and revives their memory potential by impeding epigenetic progression of Tprog cells towards terminal exhaustion. This is based on three observations: (1) Blocking de novo DNA methylation in persistently stimulated CD8 T cells skews their open chromatin landscape toward a functional signature and preserves their rejuvenation responses after ICB is discontinued. (2) Therapeutic KDM5B blockade reactivates effector and memory programs in dysfunctional human T cells in vitro, and (3) enhances TEX cell responses to ICB therapy in vivo.

### Strategy

Discussed herein is exploiting the selective killing power of CD8 T cells by either rejuvenating exhausted cells with ICB therapy, or by re-directing their specificities with chimeric antigen receptors (CAR) to attack cancer cells expressing tumor antigens. Yet, terminal T cell-exhaustion remains a major obstacle to these immunotherapies. During persistent TCR (T cell receptor) stimulation, cytotoxic T cells progressively lose their effector functions, transitioning through a less-exhausted state, including the “stem-like” or “progenitor” and “cytolytic” subsets that remain ICB-responsive, before they become terminally exhausted and refractory to ICB therapy. This progression to terminal exhaustion may explain why many patients relapse and fail to mount durable ICB responses. In addition, the therapeutic efficacy and in-vivo persistence of CAR T cells is limited, in part, due to their progression toward exhaustion. Thus, a better understanding of the molecular mechanisms underlying terminal T cell-exhaustion is crucial to boost T cell immunotherapies.

Epigenetic programming is a fundamental mechanism that regulates gene expression and cell fate commitment. TEX cells acquire distinct epigenetic programs that lock them in a dysfunctional state. Examined herein are how these epigenetic changes initiated and orchestrated in TEX cell subsets whether epigenetic programming can be blocked or reversed in Tterm cells to a functional state. Bridging these gaps allows targeting and identification of factors that apply “epigenetic brakes” to CD8 T cell function.

Exhaustion-associated epigenetic scarring remains a major barrier to rejuvenation efforts of TEX cells for treating chronic viral infections or cancer. The work was the first to reveal that the de novo DNA methyltransferase 3a (Dnmt3a) enforces terminal T cell-exhaustion by epigenetically silencing key genes that regulate T cell functions. Discussed herein is an approach to enhance the efficacy of ICB therapy through targeted inhibition of T cell-intrinsic de novo DNA methylation. Genetic deletion of Dnmt3a in activated CD8 T cells or therapeutic erasure of DNA methylation programs by decitabine treatment synergizes with ICB therapy to rescue the response of TEX cells. Yet, decitabine is a non-selective, highly cytotoxic drug that has adverse effects on other immune cells. The goal herein was to identify key factors regulating de novo epigenetic programs that drive CD8 T cells to terminal exhaustion, and to target these factors for enhancing T cell immunotherapies. Instead, the data (described below) reveal novel epigenetic regulators of T cell exhaustion and suggest an innovative approach to enhance ICB response by blocking progression towards terminal exhaustion (FIG. 1). Discussed herein is the role of these regulators in promoting exhaustion-associated de novo epigenetic changes, which will provide insights into novel strategies to unlock the efficacy and durability of T cell immunotherapies.

KDM5A/B-mediated loss of H3K4me3 modifications primes de novo DNA methylation programming in terminally exhausted CD8 T cells: Dnmt3a-mediated epigenetic programs enforce terminal exhaustion of chronically stimulated CD8 T cells and restrain their response to ICB. data reveal: a) an role of the permissive H3K4me3 marks in inhibiting the Dnmt3a activity; b) a significant loss of H3K4me3 modifications in Tterm cells; and c) a remarkable maintenance of H3K4m3 marks at effector/memory genes in Tcyto cells. These findings imply an intricate regulatory crosstalk between H3K4me3 modifications and de novo DNA methylation, that ensures silencing of both function and reprogramming capacity of CD8 T cells as they progress towards terminal exhaustion. This data support the conceptually innovative hypothesis that loss of H3K4me1/3 marks enables de novo DNA methylation programming that skews CD8 T cell differentiation towards terminal exhaustion and offsets the generation of the cytolytic subset.

Blocking epigenetic programming of exhausted T cells will enhance the efficacy and durability of T cell-immunotherapies: the and data indicate that: a) inhibiting de novo DNA methylation synergizes ICB responses and promotes durable rejuvenation after stopping ICB treatment; b) increased KDM5A/B expression is coupled with terminal dysfunction; while c) therapeutic KDM5A/B inhibition rescues effector function in chronically stimulated human CD8 T cells. Based on these findings, targeting KDM5A/B activity prevents Dnmt3a-mediated silencing of functional programs in Tterm cells. Thus, the selective KDM5A/B inhibitor treatment will provide an innovative therapeutic strategy to hinder de novo DNA methylation programming in TEX cells, rescuing their responses and promoting long-lived immunity following ICB therapy.

The in-vitro model of terminal T cell-dysfunction to study epigenetic (re)programming in persistently stimulated human CD8 T cells, discussed herein, establishes a stable dysfunctional program that contrasts with other in vitro CAR T cell systems. These models produced only transient dysfunction of CAR T cells, which was reversible upon resting. Thus, the tractable model will facilitate mechanistic investigations of human terminal T cell-dysfunction and guide the complementary in vivo animal studies. Used herein are state-of-the-art multi-omics to profile both global DNA methylation and histone H3K4 methylation in small numbers of CD8 T cell subsets, revealing the spectrum of epigenetic programs within TEX cell subsets. The innovative approach will provide invaluable insights into CD8 T cell functional plasticity and responsiveness to ICB therapy.

### Approach

Terminal exhaustion is a major challenge to T cell immunotherapies. Therefore, it is useful to identify cell-intrinsic barriers for rejuvenation of Tterm cells during chronic infections or cancer. Discussed herein is a unique approach to block or reverse progression towards the terminally exhausted state by targeting epigenetic modifiers.

### De Novo DNA Methylation Contributes to Terminal T Cell Exhaustion.

Dnmt3a-mediated de novo DNA methylation plays a role in establishing T cell exhaustion. Conditional deletion of Dnmt3a (Dnmt3a cKO) in activated CD8 T cells blocks their progression towards terminal exhaustion.

Furthermore, Dnmt3a-deficient antigen-specific CD8 T cells are exhaustion-resistant during lifelong chronic lymphocytic choriomeningitis virus (LCMV) infection—an animal model of severe T cell exhaustion. At the mechanistic level, virus-specific cKO CD8 T cells were maintained at higher numbers in lymphoid and non-lymphoid tissues, retained high capacity to recall effector cytokines (e.g., IFNg, IL-2, TNF), and continued to express higher levels of stemness-associated transcription factors, such as Tcf1 and Lef1, relative to the wild type (WT) CD8 T cells, despite prolonged TCR stimulation and high PD-1 expression. This data established de novo DNA methylation as a novel, cell-intrinsic mechanism that is causally linked to T cell exhaustion.

### De Novo DNA Methylation Enforces Silencing of Effector and Stemness Programs in TEX Cells Through Remodeling the Open Chromatin Landscape.

TEX cells undergo distinct changes in global chromatin accessibility and histone modifications during chronic viral infections or cancer. Furthermore, exhaustion-specific open chromatin landscape can be maintained in the absence of antigen, suggesting an underlying cell-intrinsic mechanism. To better understand the heritable nature of exhaustion-specific programs, profiled herein are whole-genome DNA methylation changes in antigen-specific WT and Dnmt3a-cKO CD8 T cells during acute and chronic LCMV infections on day 35 post-infection (p.i). Cells progressively acquired >2000 newly methylated regions during naive-to-effector-to-exhausted transitions. Many of these differentially methylated regions (DMRs) were acquired in genes that regulate T cell function, metabolic activity and self-renewal, such as Ifng, Tbx21 (encoding T-bet), Myc, and Tcf7 (encoding Tcf1). Profiled herein are chromatin accessibility changes in antigen-specific WT and Dnmt3a cKO CD8 T cells during chronic LCMV infection to determine whether Dnmt3a-mediated epigenetic programming regulates the open chromatin landscape of TEX cells. DNA methylation and chromatin accessibility changes are inversely linked, with >60% of exhaustion-associated open chromatin regions (OCRs) regulated by Dnmt3a; The majority of Dnmt3a-regulated OCRs are acquired at the post-effector stage; and cKO CD8 T cells retain chromatin openness at many effector function and stemness-related genes relative to WT exhausted cells (FIG. 2). Overall, these findings indicate that de novo DNA methylation serves as a T cell-intrinsic mechanism that, in part, shapes the open chromatin landscape enforcing terminal exhaustion.

The Role of the H3K4 Demethylases KDM5A/B in Regulating De Novo DNA Methylation Programs that Enforce Terminal Exhaustion.

The data show that Dnmt3a-mediated DNA methylation plays a crucial role in establishing T cell-exhaustion, but it is not yet clear if specific histone modifications control the initiation of DNA methylation programming. H3K4me3 and H3K4me1 are chromatin hallmarks of active promoters and enhancers, respectively. Furthermore, MLL1-dependent H3K4me3 programming was useful for promoting the stemness features of antigen-experienced CD8 T cells. biochemical and structural studies indicate that unmethylated H3K4 residues activate the DNMT3A enzyme.

H3K4me3 marks are more enriched in Tcyto cells than in Tterm cells, the H3K4 histone demethylases, KDM5A/B, are downregulated in the Tcyto subset but maintained in Tterm cells, and H3K4me3 enrichment generally inhibits Dnmt3a-mediated DNA methylation activity. Yet, it remains unknown whether the loss of H3K4me1/3 modifications primes chronically stimulated CD8 T cells for terminal exhaustion by enabling de novo DNA methylation programming. To better understand this epigenetic crosstalk, employed herein are cutting-edge approaches, including the tractable in vitro model of human T cell dysfunction and a complementary in vivo model of chronic LCMV infection. Profiled herein dynamic changes in H3K4me1/3 levels and DNA methylation programs as CD8 T cells progress towards terminal dysfunction. the integrative analyses of these epigenetic changes will allow us to determine whether: 1a) de novo DNA methylation tracks with an initial loss of H3K4me1/3 at effector and memory-associated genes in mouse and human TEX cells; 1b) targeting KDM5A/B in chronically stimulated CD8 T cells preserves H3K4me1/3 marks and hinders exhaustion-associated DNA methylation; 1c) KDM5A/B-deficient CD8 T cells maintain chromatin accessibility and transcriptional activity of T cell functional programs; and 1d) unique transcription factors regulate epigenetic routing towards Tcyto versus Tterm. Discussed herein is the direct interplay between histone H3K4me1/3 and de novo DNA methylation orchestrates terminal exhaustion.

### Terminally Exhausted T Cells Lose H3K4Me3 Marks and Undergo De Novo DNA Methylation at Effector Memory-Related Loci.

To determine whether changes in histone H3K4me3 modifications correlate with de novo DNA methylation and subsequent chromatin accessibility changes in TEX cell subsets, integrated herein are the H3K4me3 profiles of LCMV-specific TEX subsets with the DNA methylation datasets and open chromatin landscapes of naive and chronically stimulated Dnmt3a-cKO or WT LCMV-specific CD8 T cells, as well as chromatin accessibility profiles of LCMV-specific Tprog cells or Cxcr5− Tim-3+CD8 T cells isolated from chronically LCMV-infected animals (FIG. 3). Tterm cells lose H3K4me3 modifications at effector and memory-related genes, such as Tbx21, Tcf7, Lef1, Ifng, and Myc. these changes in H3K4me3 modifications were coupled with de novo DNA methylation programs and reduced chromatin accessibility in WT TEX cells (examples are shown in FIG. 3). In contrast, chronically stimulated Dnmt3a-deficient cells retained chromatin openness at these loci-patterns similar to those in the Tprog and Tcyto subsets. At the global level,

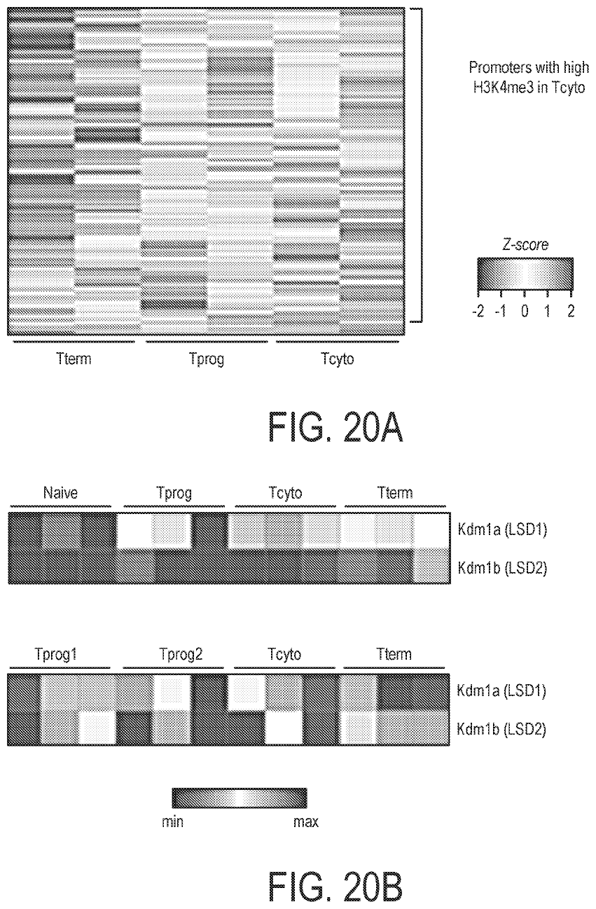

H3K4me3 marks were preserved at the promoters of numerous effector genes within Tcyto cells. However, they were absent in Tterm cells and partially reduced in Tprog cells (FIG. 20A). This data indicate that Tterm cells lose H3K4me3 marks and undergo de novo DNA methylation, enforcing chromatin inaccessibility and silencing of memory T cell programs. While changes in H3K4me1/2 marks remain to be investigated in TEX subsets, it is unknown whether preserving H3K4me1-3 marks can prevent de novo DNA methylation events that drive terminal exhaustion.

### Cytolytic CD8 T Cells Downregulate Specific H3K4 Demethylases.

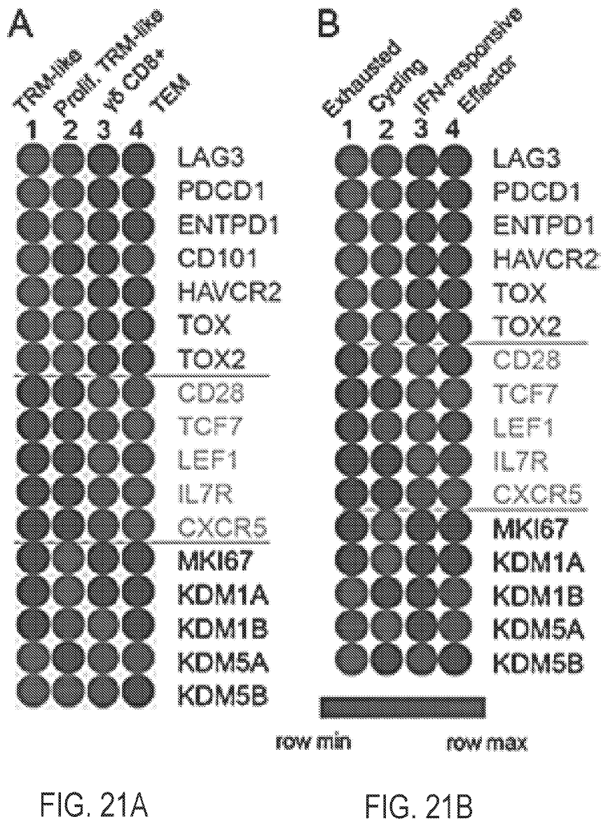

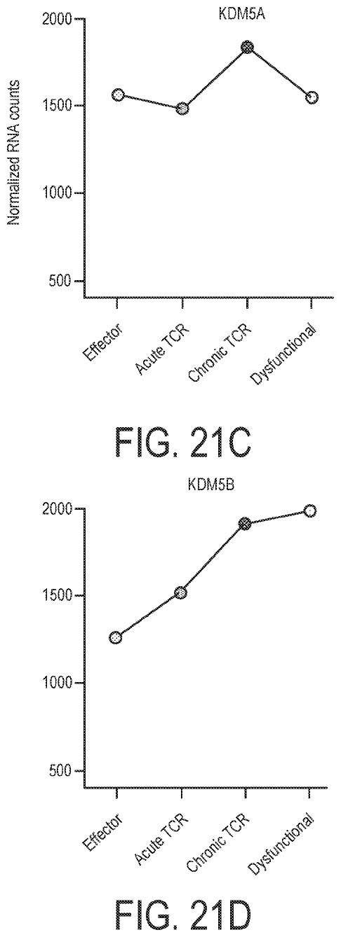

To better understand factors that affect H3K4 methylation changes in TEX cells, examined herein are the expression levels of key H3K4 demethylases in TEX cell subsets. the expression levels of Kdm1a and Kdm1b are similar among TEX subsets (FIG. 20B). However, Kdm5a is upregulated in the Tterm subset, while both the Tprog and Tterm cells maintained elevated expression of Kdm5b (FIG. 20C-F). In contrast, Tcyto cells and the intermediate subset “Tprog2” downregulate both Kdm5a and Kdm5b (FIG. 20C-F). Furthermore, functional memory human CD8 T cells have lower expression levels of KDM5B, but not KDM5A, compared to dysfunctional tumor reactive CD8 T cells within breast cancer (FIG. 21A) or brain metastases from melanoma patients (FIG. 21). Similarly, the in vitro model of human T cell dysfunction shows that effector human CD8 T cells exhibit reduced levels of KDM5B, while maintaining comparable levels of KDM5A, relative to dysfunctional CD8 T cells that arise under conditions of chronic TCR plus TGF(31 stimulation (FIG. 21C,D). The variations observed in the expression patterns of KDM5A between mouse and human CD8 T cell subsets highlight the need to discern its relative contribution to the progressive loss of H3K4me3 modifications as CD8 T cells undergo terminal exhaustion. KDM5B enzyme can fully erase H3K4 methylation by demethylating tri-, di-, and mono-methylated H3K4 residues resulting in unmethylated H3K4 tails, which activate DNMT3A enzyme.

In contrast, KDM5A enzyme can demethylate H3K4me3/me2 marks only resulting in H3K4me1, which remains as a permissive mark enriched in active enhancers and gene regulatory elements and retains the ability to inhibit de novo DNA methylation events. the majority of exhaustion-associated de novo DNA methylation events are acquired at enhancers and distal regulatory elements rather than promoter regions. Overall, these data suggest that KDM5B, and possibly KDM5A, play an important role in orchestrating DNMT3A-mediated epigenetic progression towards terminal exhaustion by removing H3K4me1-3 marks. As such, it is imperative to determine the differential role of KDM5A and KDM5B in regulating the epigenomes of TEX subsets.

Cytolytic CD8 T Cells Maintain their Effector Functions Through Cell-Intrinsic Epigenetic Mechanisms.

TEX subsets spatially organize in distinct niches. In tumor settings, Tprog cells are enriched within the tumor-draining lymph nodes, tertiary lymphoid structures or tumor periphery, while Tcyto and/or Tterm subsets are more localized within the tumor parenchyma. Similarly, virus-specific CD8 T cell subsets are spatially organized in chronically LCMV-infected animals, with Tprog cells residing primarily in the splenic white-pulp, while PD-1+Cxcr5-Tcf1-cells, including Tcyto and Tterm cells, preferentially migrate to the peripheral tissues or the splenic red-pulp where viral antigen loads are high. These intriguing findings suggest that, despite Tcyto and Tterm cells being exposed to comparable levels of antigens and/or signals from the microenvironment, Tcyto cells retain their cytotoxic and effector functions through stable cell-intrinsic mechanisms. Indeed, Tcyto cells have distinct histone methylation maps and open chromatin landscape compared to Tterm cells.

De Novo DNA Methylation Tracks with an Initial Loss of H3K4Me1 3 at Effector and Memory-Associated Genes in Mouse and Human TEX Cells.

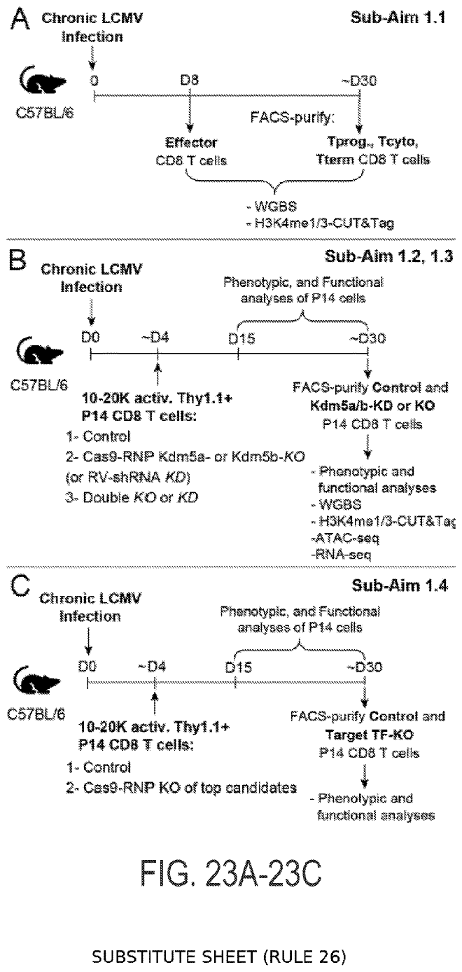

The integrating epigenetic analysis demonstrate that Tterm cells undergo H3K4 demethylation at effector and memory-related genes that also gain DNA methylation programs and lose chromatin accessibility within WT TEX cells. To rigorously define the temporal role of H3K4me1/3 changes in regulating the progressive acquisition of DNA methylation programs during the development of terminal exhaustion, performed herein are CUT&Tag-sequencing of H3K4me1 and H3K4me3 marks and whole-genome bisulfite sequencing (WGBS) assays of virus-specific effector CD8 T cells and TEX subsets (Table 1), isolated from spleens of chronically LCMV-infected animals on days 8 and 30 post-infection (p.i), respectively (FIG. 23A).

Discussed herein are H3K4me1/3 changes in acutely stimulated or chronically stimulated dysfunctional human CD8 T cells. These epigenetic profiles will reveal the spectrum of progressive changes in H3K4me1/3 and DNA methylation programs within effector CD8 T cells as they develop terminal dysfunction. These data will pinpoint the temporal impact of H3K4me1/3 changes on CD8 T cell's de novo DNA methylation programming that drives and enforces a terminal dysfunction fate.

The integrative epigenetic analyses will reveal an inverse relationship between H3K4me1/3 and DNA methylation programs at gene regulatory elements of effector and memory-related loci within mouse and human CD8 T cells as they develop terminal dysfunction. It is possible that Tprog cells undergo initial H3K4 demethylation at genes that later acquire DNA methylation events during cell proliferation and differentiation to terminal exhaustion. To dissect these epigenetic events, alternatively isolate herein are Tprog cells from chronically LCMV-infected animals on day 28 p.i and adoptively transfer them into congenically distinct, infection-matched animals. After 7-10 days, isolated and profiled herein are H3K4me1/3 and DNA methylation changes in the adoptively transferred cells that develop a Tterm phenotype under chronic stimulation settings. Tracking epigenetic changes pre- and post-adoptive transfer will reveal the kinetics of H3K4me1/3 loss as Tprog cells acquire DNA methylation programs that enforce terminal exhaustion. Used herein is a CD4-depleted chronic LCMV infection model in these settings to ensure the lifelong persistence of viral antigens.

### Targeting KDM5A B in Chronically Stimulated CD8 T Cells Preserves H3K4Me1/3 Marks and Hinders Exhaustion-Associated DNA Methylation.

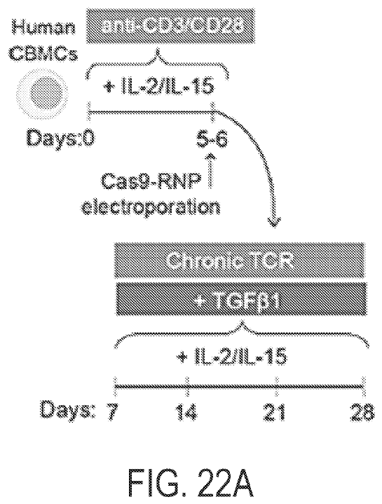

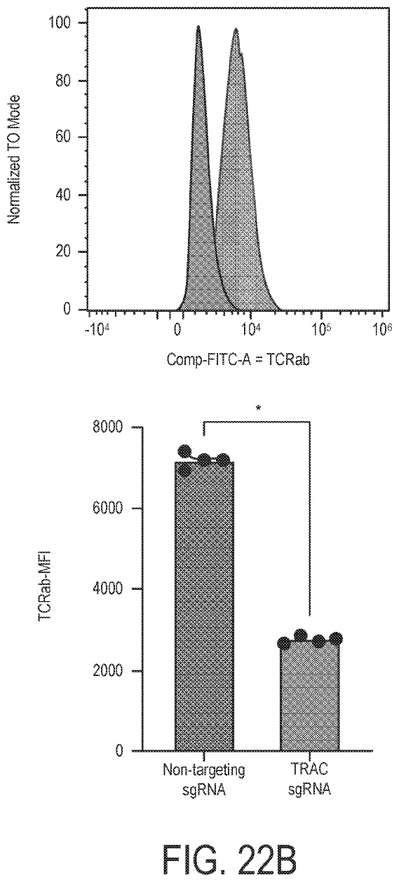

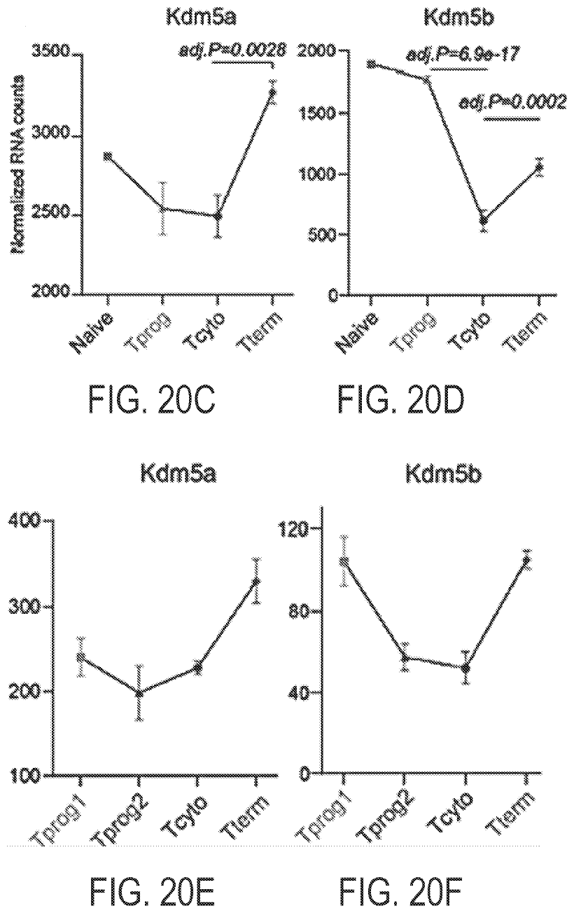

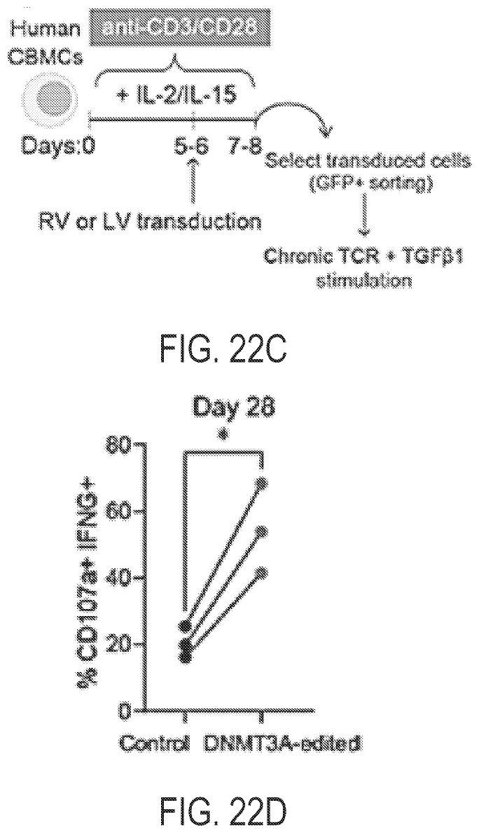

The highly cytolytic subset of TEX cells express lower levels of KDM5A/B relative to Tterm cells, suggesting a negative impact of KDM5A/B on CD8 T cell functions. To determine whether the activity of KDM5A/B enzymes contribute to the initiation of exhaustion-associated DNA methylation programming, (KO)KDM5A or KDM5B are knocked out in ex vivo-stimulated, cord blood-derived human CD8 T cells using the optimized Cas9-RNP electroporation protocol (day 7)—example is shown for knocking out TRAC gene in human CD8 T cells to confirm the feasibility (FIG. 22A,B). Then, the genetically modified or control CD8 T cells will be maintained under chronic TCR plus TGF(31 stimulation conditions that establish terminal dysfunction. On day 28, performed herein is H3K4me1/3 CUT&Tag-seq and WGBS of KO and control human CD8 T cells. Similarly, Kdm5a or Kdm5b are KO in ex vivo-stimulated TCR-transgenic mouse gp33-specific P14 CD8 T cells using the optimized in vitro model of mouse T cell exhaustion, followed by adoptive transfer of control or Cas9-edited Thy1.1+P14 cells into chronically LCMV-infected C57BL/6 animals. On day 30 p.i, discussed herein is the isolation of P14 cell subsets and the assessment of changes in their H3K4me1/3 and DNA methylation epigenomes (FIG. 23B). Comparing DNA methylation changes in chronically stimulated KO versus control CD8 T cells will reveal the impact of modulating H3K4me1/3 levels on exhaustion-specific DNA methylation programs.

Targeting KDM5A or KDM5B will preserve H3K4me3 or H3K4me1/3 marks, respectively, in mouse and human CD8 T cells, which will prevent the acquisition of exhaustion-specific de novo DNA methylation programs. It is possible that knocking out KDM5A or KDM5B decreases the survival of Cas9-edited CD8 T cells under in vitro chronic stimulation settings. As an alternative approach, KDM5A/B expression is downregulated using the shRNA-retroviral transduction approach. The RV-transduction protocol in human CD8 T cells (example is shown in FIG. 22C,D) was successfully optimized. It is also possible that KDM5A and KDM5B exhibit redundant enzymatic activity in the gradual loss of H3K4me3 marks during T cell exhaustion. When such redundancy is observed, both enzymes are double-KO or KD in CD8 T cells to enhance the preservation of H3K4me1/3 marks.

### KDM5A B-Deficient CD8 T Cells Maintain Chromatin Accessibility and Transcriptional Activity of T Cell Functional Programs.

The findings establishes a causal role of de novo DNA methylation in enforcing a terminal dysfunctional state in chronically stimulated murine and human CD8 T cells (FIG. 22D). To determine whether targeting H3K4 demethylation curbs the epigenetic progression of CD8 T cells to terminal exhaustion, changes were tracked in effector functions and cytolytic activity of KDM5A- or KDM5B-KO and control human CD8 T cells under chronic TCR+TGF(31 stimulation, quantifying cytokine production (e.g., IFNG, TNF, IL-2) after PMA-stimulation, and the levels of degranulation marker CD107a, TBET, Perforin, Granzyme B, and Kᵢ-67, as well as the expression of memory-related molecules TCF1 and CD28 by multi-color spectral cytometry. In addition, the genome-wide effects of targeting KDM5A or KDM5B on chromatin accessibility and transcriptional activity of human T cell programs were elucidated by performing ATAC-seq and RNA-seq, respectively, on day 28. Discussed herein is assessing phenotypic and functional changes in the adoptively transferred KO or control P14 cells from chronically LCMV-infected animals on day 30 p.i by measuring: a) degranulation and effector cytokine production (e.g., CD107a, IFNg, TNF, IL-2) after ex vivo stimulation with the gp33 peptide; b) quantities and distribution of TEX subsets within secondary lymphoid (spleen) and peripheral tissues (liver, lungs, and blood) on day 30 p.i; and c) proliferation activity and expression levels of sternness-associated markers (e.g., Kᵢ67, Tcf1, CD62L) and surface inhibitory receptors (e.g., PD-1, Lag3, Tim-3, 2B4, . . . etc). Furthermore, KO or control P14 cells were FACS-purified from spleens of chronically LCMV-infected animals on day 30 p.i for ATAC-seq and RNA-seq assays (FIG. 23B). Integrating these comprehensive epigenetic maps and transcriptional networks in TEX subsets will reveal unique regulators that mediate epigenetic routing towards Tcyto versus Tterm cells.

Targeting KDM5A/B will prevent terminal dysfunction of chronically stimulated human or mouse CD8 T cells. Therefore, KDM5B (or KDM5A)-deficient human or mouse CD8 T cells will maintain their effector functions and proliferation activity under chronic stimulation settings. The enhanced effector functions in KO versus control T cells will be coupled with increased chromatin accessibility and transcription of effector and memory-associated genes. It is possible that redundant activities of KDM5A and KDM5B in CD8 T cells result in partial recovery of effector functions. Alternatively, both enzymes in CD8 T cells were double-KO or KD and changes measured in T cell functions and chromatin accessibility.

Unique Transcription Factors that Regulate Epigenetic Routing Towards Tcyto Versus Tterm.

Although Tcyto and Tterm cells are exposed to comparable levels of chronic antigens and microenvironmental signals, Tcyto cells preserve their functionality and responsiveness to ICB therapy, reflecting distinct transcriptional programming in TEX subsets. TFs play a role in regulating some aspects of TEX differentiation (e.g., TCF1, BACH2, NFAT, TOX, RUNX3, BATF, IRF4, IRF7, KLF2). Discussed herein are which TFs shape the epigenetic histone-DNA methylation reprogramming of Tprog cells towards the Tcyto versus Tterm states. The multiomics datasets herein are used to identify TFs with motifs enriched in differentially H3K4/DNA-methylated and accessible regions in Tcyto versus Tterm cells (i.e., defined by increased H3K4me1/3 deposition, DNA-hypomethylation, and chromatin openness). These TFs will be ranked and prioritized based on their prevalence, significance of motif enrichment, and transcriptional activity in TEX subsets. Discussed herein are the leading 5-10 TF candidates from each subset and determine their role in the epigenetic regulation and differentiation of TEX subsets. Leading candidates were evaluated by employing the Cas9-RNP gene-editing approach using 4-6 sgRNAs/target in ex vivo stimulated P14 cells. Control or edited P14 cells are adoptively transferred into chronically LCMV-infected animals (FIG. 23C). On day 30 p.i, quantified herein is the distribution of the P14 subsets and isolate them for sequencing analysis to identify the gene edits enriched in each TEX subset.

Targeting the top TFs enriched in regions with increased H3K4me1/3 and DNA-hypomethylation will skew the differentiation trajectory towards either the Tcyto or Tterm state based on their role in the epigenetic remodeling. For example, targeting TFs that promote the maintenance of H3K4me1/3 modifications will shift the differentiation to a terminal exhaustion state, which will be coupled with reduced effector functions of the edited P14 cells. In contrast, targeting TFs that facilitate KDM5A/B recruitment and binding to gene regulatory elements in Tterm cells will reduce the erasure of H3K4me1/3 modifications, thereby maintaining a functional state in CD8 T cells. It is possible that targeting specific TF(s) impacts the early expansion of the Tprog precursor cells. To address this possibility, utilized herein is the tet-inducible Cas9- or shRNA-RV transduction system in P14 cells to knock out or downregulate these TFs at the late phase of the chronic infection (~day 21 p.i) and assess changes in the differentiation of TEX subsets.

Targeting KDM5A/B H3K4 Demethylases in TEX Cells Enhances their ICB Responses and Epigenetically Revives their Memory Potential.

The data indicate that blocking de novo DNA methylation in CD8 T cells renders them exhaustion-resistant and promotes durable responses to ICB therapy. Further discussed herein is the role of KDM5A/B in terminal T cell-exhaustion. Discussed herein are therapeutic efficacy of targeting KDM5A/B activity in TEX cells for enhancing ICB responses and the molecular mechanism underlying this new therapeutic strategy.

The data show that late treatment with a small molecule inhibitor of KDM5A/B recovers effector and memory programs in dysfunctional human CD8 T cells. This treatment also improves their cytotoxic activity in vitro and enhances ICB responses during chronic viral infection in vivo. Selective H3K4 demethylase inhibitor treatment shifts differentiation of Tprog cells to the cytolytic, rather than terminally exhausted state, thereby enhancing T cell responses to ICB therapy. Discussed herein are the therapeutic benefits of a selective KDM5A/B inhibitor using preclinical models of T cell exhaustion, as well as adoptive transfer experiments to determine whether targeting KDM5A/B: 2a) skews Tprog cell differentiation towards the cytolytic state by curbing epigenetic progression towards terminal exhaustion; 2b) enhances TEX responses against solid tumors during ICB therapy; and 2c) enables durable T cell rejuvenation by reviving their recall responses and memory potential.

Blocking De Novo DNA Methylation in CD8 T Cells Preserves their Potential for Rejuvenation after Cessation of ICB.

Many patients fail to develop durable protection against tumor relapses after ICB treatment is discontinued. Therefore, it is crucial to develop complementary therapeutic strategies for generating durable rejuvenation of TEX cells. The work demonstrates that blocking de novo DNA methylation in TEX cells substantially prolongs their proliferation activity after discontinuation of ICB treatment. In contrast to WT

CD8 T cells, Dnmt3a-deficient cells were maintained at significantly higher numbers during lifelong chronic LCMV infection and exhibited high proliferative capacity for >1 month after cessation of ICB treatment (FIG. 5). These findings indicate that blocking epigenetic programs within TEX cells may provide an effective strategy to preserve the proliferative response and memory potential after ICB therapy.

Therapeutic Targeting of KDM5A B Recovers Effector and Memory Programs in Dysfunctional CD8 T Cells and Enhances their ICB Responses.

To test the therapeutic efficacy of targeting KDM5A/B in dysfunctional CD8 T cells, chronically TCR plus TGFβ1-stimulated human T cells were treated with the selective inhibitor of KDM5A/B, GSK467 (25 μM), on days 14-28 with or without TGFBR1 blocker. While TGFβ1 blockade recovered effector functions of chronically stimulated CD8 T cells, KDM5A/Bi treatment further potentiated the reactivation of polyfunctionality and memory expression programs, and augmented tumor-killing activity (FIG. 6A-6G). KDM5A/Bi monotherapy significantly restored effector cytokine secretion and expression of memory-related transcription factors in dysfunctional human CD8 T cells (FIG. 6A-6G). Also discussed herein is the efficacy of KDM5A/Bi therapy during lifelong chronic LCMV infection in vivo. late KDM5A/Bi treatment (days 25-33 p.i) enhanced the responses of virus-specific TEX cells to PD-L1 blockade, with significant expansion of both Tprog and Tcyto cells (FIG. 7A-7E). These exciting findings demonstrate the therapeutic efficacy of KDM5A/B blockade to enhance the rejuvenation of TEX cells.

### Targeting KDM5A B Skews Tprog Cell Differentiation Towards the Cytolytic State by Impeding Exhaustion-Associated DNA Methylation.

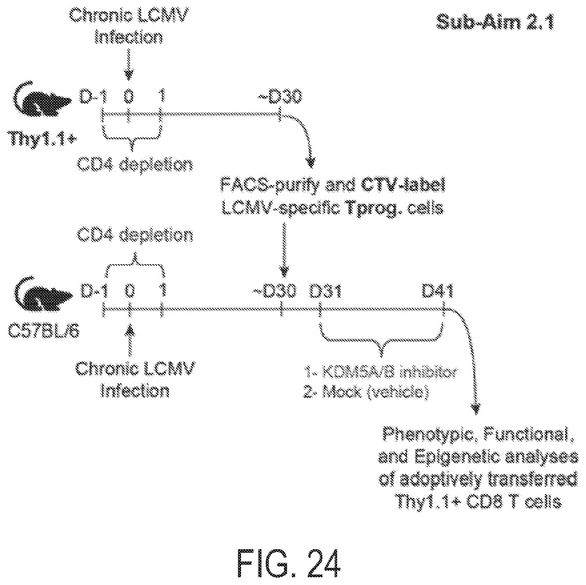

The data showed that KDM5A/B inhibition can potentiate the recovery of effector functions of chronically stimulated human CD8 T cells in vitro. Also discussed herein is whether a selective KDM5A/Bi treatment can enhance CD8 T cell responses by blocking their epigenetic progression towards terminal exhaustion in vivo using CD4-depleted chronic LCMV infection model. On day 30 p.i, isolated herein is virus-specific Thy1.1+Tprog cells, followed by CTV-labeling and adoptive transfer into infection-matched C57BL/6 animals. KDM5A/Bi or vehicle treatment was started on days 31-41 p.i (FIG. 24). Following treatment regimen, quantity, phenotype, proliferative potential, and functional capacity of adoptively transferred CD8 T cells was measured using spectral cytometry, as described herein. In addition, Oxford Nanopore-seq technology was used to measure DNA methylation levels at exhaustion-specific DMRs in the transferred CD8 T cells, following a protocol previously optimized for small cell numbers in the laboratory. These data will reveal the effects of therapeutic KDM5A/B blockade on the Tprog-to-Tterm differentiation at the functional and epigenetic levels.

KDM5A/Bi treatment will increase the quantity of Tcyto cells while impeding the development of Tterm cells by decreasing the acquisition of de novo DNA methylation events at effector and memory genes. The in vitro experiments indicate that KDM5A/Bi treatment enhances the cytolytic activity and effector functions of dysfunctional human T cells through direct effects on CD8 T cells (FIG. 6A-6G). The effects observed in vivo after systemic KDM5A/Bi treatment may be influenced not only by modulating CD8 T cells but also by affecting other immune cells, such as regulatory or follicular helper CD4 T cells. To address this possibility, Tprog cells are alternatively transferred into infection-matched Rag1-KO mice which lack T and B cells, followed by KDM5A/Bi therapy. This approach will determine whether the therapeutic benefits of KDM5A/B inhibition are CD4 T cell independent.

### Selective KDM5A Bi Treatment Enhances TEX Responses Against Solid Tumors During ICB Therapy.

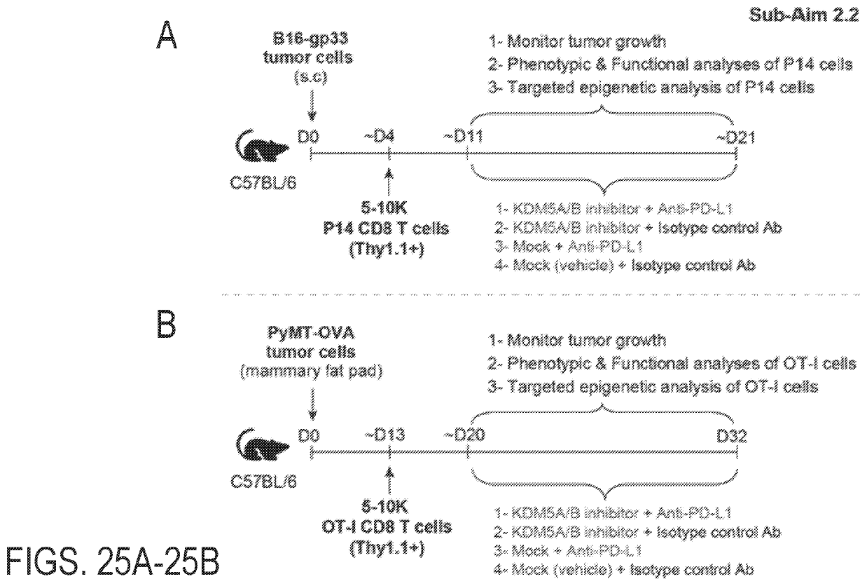

The data showed that therapeutic blockade of KDM5A/B enzymes potentiates the TEX responses to ICB therapy during chronic viral infection. Targeting KDM5B in melanoma cells leads to enhanced tumor rejection by derepressing the expression of endogenous retroviral elements in tumor cells and reducing immune evasion of tumor cells. These findings provide a strong rationale for testing the in vivo efficacy of a selective KDM5A/B inhibitor as an adjunctive treatment to ICB therapy in ICB-refractory tumors. Discussed herein is whether the pharmacological inhibition of KDM5A/B recovers the responses of anti-tumor CD8 T cells to ICB treatment during solid tumors. Utilized herein are two tumor models known to be refractory to PD-1/PD-L1 blockade therapy, including orthotopic OVA-expressing PyMT breast adenocarcinoma and GP33-expressing B16-F10 melanoma in C57BL/6 animals. One week following the adoptive transfer of Thy1.1+TCR-transgenic CD8 T cells (OVA-specific OT-I or GP33-specific P14 cells) into tumor-bearing animals, mono- or combined therapy using KDM5A/Bi plus anti-PD-L 1 treatment was started (FIG. 25A-25B). Changes in tumor growth was tracked and measure the quantity, phenotype, proliferative potential, and functional capacity of adoptively transferred anti-tumor CD8 T cells measured within tumors and draining lymph nodes using spectral cytometry as described herein. In addition, tumor-infiltrating CD8 T cells are isolated and Oxford Nanopore-seq technology used to measure DNA methylation levels at exhaustion-specific DMRs, as described above.

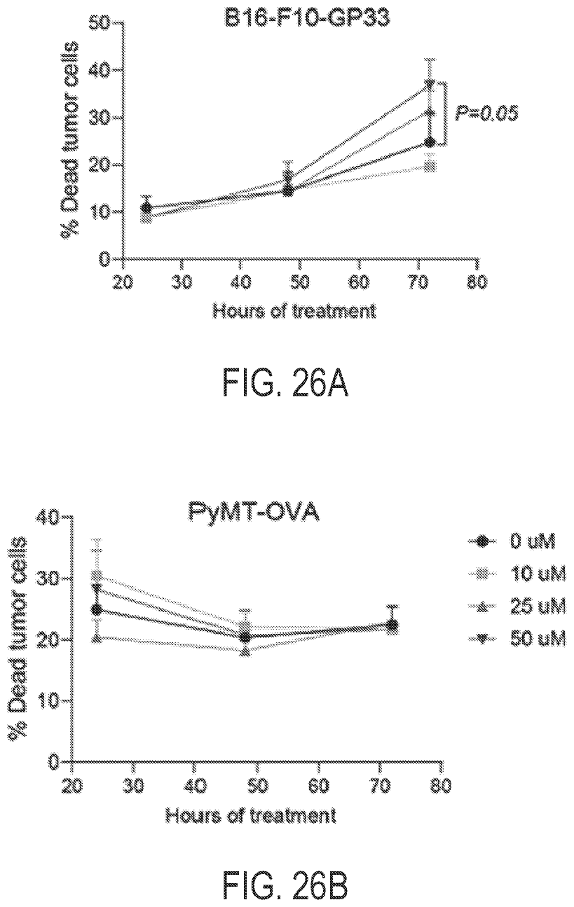

Selective KDM5A/B inhibitor therapy will recover the responses of anti-tumor CD8 T cells to ICB treatment leading to significant tumor control in combined treated animals. It is possible that these effects are driven, in part, by KDM5A/Bi-mediated effects on tumor cell growth or survival. Indeed, the data indicate that short-term KDM5A/B inhibitor treatment increases the frequencies of dead B16 melanoma cells in vitro (FIG. 26A-26B), consistent with the role of KDM5B in promoting the growth of melanoma cells. While the potential effects of KDM5A/Bi therapy on tumor growth remain beneficial, discussed herein are the relative effects of KDM5A/Bi treatment on CD8 T cells versus tumor cells through the adoptive transfer of control or KDM5A/B-KO P14 cells (generated as described herein) into B16-GP33 tumor-bearing animals followed by anti-PD-L1 treatment one week later. Further discussed herein are the effects of KDM5A/Bi monotherapy on tumor growth in vivo in B16 or PyMT tumor-bearing, immunodeficient NSG animals without prior transfer of tumor-specific CD8 T cells. Confirmed herein is the relative contribution of targeting KDM5A/B in tumor and CD8 T cells to the treatment outcomes.

Selective KDM5A Bi Treatment Enables Durable T Cell Rejuvenation by Reviving their Recall Responses and Memory Potential.

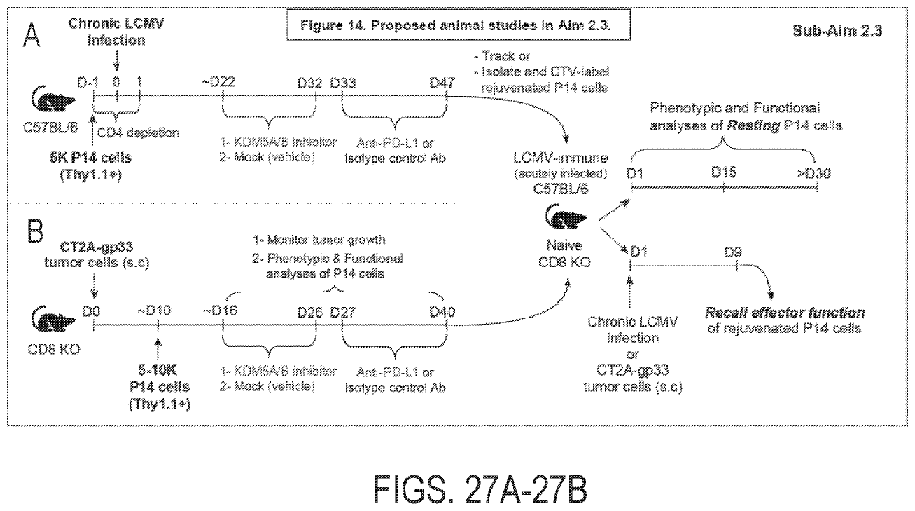

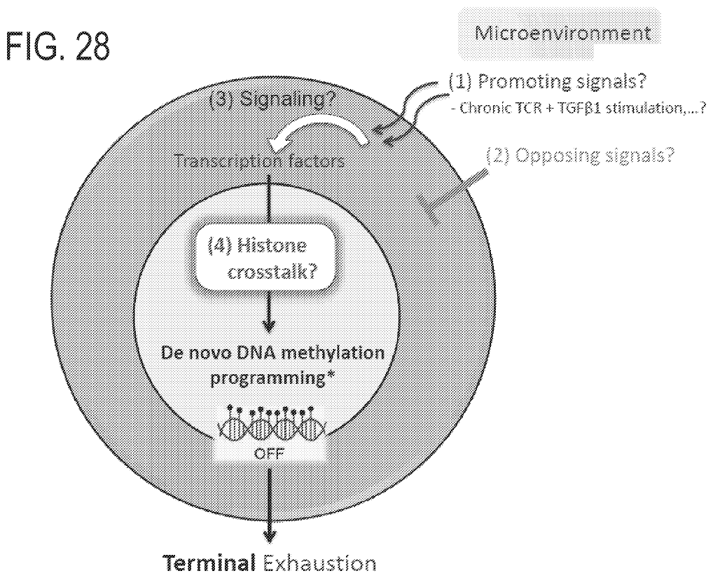

The data indicate that blocking de novo DNA methylation programming in TEX cells promotes their durable responses to ICB therapy. Discussed herein is the durability of the ICB responses in the treatment-modulated P14 CD8 T cells under both chronic LCMV infection and gp33-expressing tumor settings. Employed herein is the CT2A-GP33 glioma, rather than B16-GP33 melanoma, model as the data indicate that the CT2A-GP33 tumors have slower in vivo growth kinetics compared to that of the B16 melanoma model. Thus, the CT2A tumor model will facilitate the administration of a sequential treatment regimen, resembling the treatment plan and duration proposed for the chronically LCMV-infected animals (FIG. 27A-27B). After the administration of KDM5A/Bi therapy (days 22-32 p.i; days 16-26 post-tumor implantation), chimeric mice are treated with anti-PD-L1 for 2 weeks. changes in phenotype, function, and proliferation response of P14 cells are tracked over 1 month after cessation of PD-L1 blockade. Discussed herein is the memory potential of the rejuvenated CD8 T cells by transferring CTV-labeled, control or KDM5A/Bi-treated P14 cells after stopping ICB into LCMV-immune or tumor-naive mice. Assessed herein is their self-renewal capacity by tracking the CTV dilution of the transferred T cells. Also discussed herein is their recall effector function by challenging the recipient mice with the same tumor cells or a high dose of LCMV clone 13 (FIG. 27A-27B). Employed herein are CD8-KO animals as recipients for CT2A-GP33 tumor cells to facilitate the expansion of adoptively transferred anti-tumor P14 cells. This approach will enable the isolation of an ample number of CD8 T cells for subsequent investigations into memory and recall potential of rejuvenated CD8 T cells.

KDM5A/Bi treatment-induced reprogramming of exhausted P14 cells will synergize their proliferation response and promote long-lived memory function after ICB treatment. In some examples, KDM5A/B blockade can be extended during anti-PD-L1 treatment to protect the rejuvenated CD8 T cells from converting to the terminal-exhaustion state under settings of persistent antigen exposure. There is also a possibility that some C2TA tumors may continue growing after stopping PD-L1 blockade. Alternatively, rejuvenated CD8 T cells are isolated from tumor-bearing mice and test their memory and recall effector function by transferring them into LCMV-immune or naive mice as described above. While the in vitro experiments indicate that KDM5A/Bi treatment can recover effector and memory programs through direct modulation of human CD8 T cells (FIG. 6A-6G), it remains possible that the in vivo effects following systemic KDM5A/Bi treatment are mediated by modulating other immune cells in addition to CD8 T cells. To confirm the CD8 T cell-intrinsic function of KDM5A/B in silencing the memory potential of TEX cells, the adoptive transfer experiments are repeated using KDM5A/B-KO or KD P14 cells followed by PD-L1 blockade therapy as described above.

Discussed herein is the therapeutic significance of targeting KDM5A/B enzymes in TEX cells and provides useful insights into designing novel therapeutics that selectively target KDM5A or KDM5B enzyme in TEX cells (e.g., an anti-PD-1-KDM5A/Bi conjugation system) to block terminal exhaustion and promote durable responses to T cell-based immunotherapies.

### Example 3: H3K4 Demethylation T Cell Exhaustion

### Histone Modifications Regulate De Novo DNA Methylation

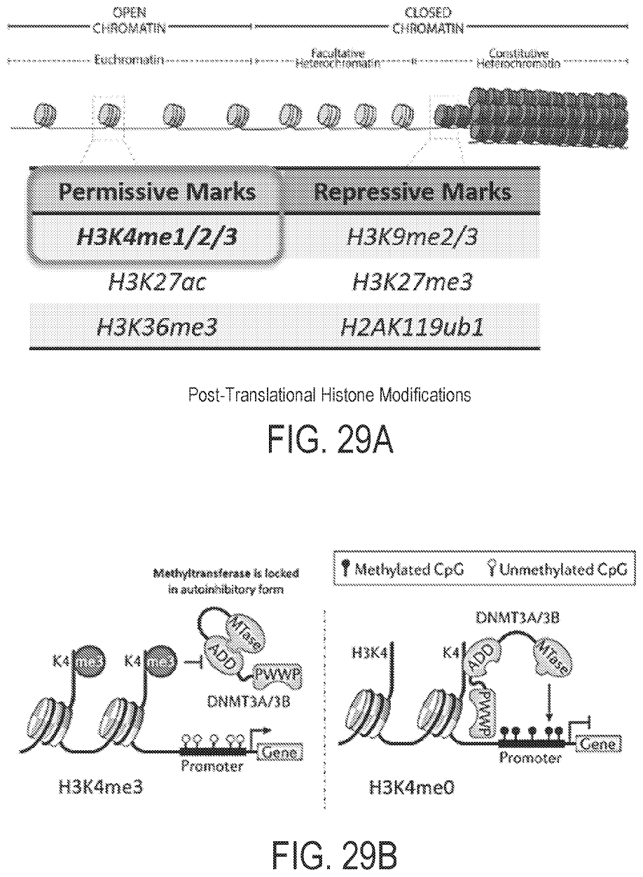

Some activating histone marks such as H3K4me3 block de novo DNA methylation. (FIGS. 29A-29B). The domain of DNA methyltransferase 3A (DNMT3A) and DNMT3B (ADD) interacts with unmodified histone H3 lysine 4 (H3K4), which relieves the autoinhibitory interaction between this domain and the methyltransferase (MTase) catalytic domain. This promotes de novo DNAme and transcriptional repression, especially at CpG island (CGI) promoters devoid of H3K4me3.

Conversely, methylation of H3K4 inhibits the interaction between this histone tail residue and the ADD domain, protecting marked CGI promoters against de novo DNAme. The ADD domain is unable to interact with methylated H3K4 and remains in its autoinhibitory state in the presence of H3K4me. The higher the H3K4 methylation state, the greater the inhibitory effect on DNMT3A/B, effectively preventing DNAme-mediated silencing of H3K4me3-marked promoters.

Loss of H3K4Me3 is Coupled with De Novo DNA Methylation in Terminally TEX Cells

There are three subsets of exhausted CD8 T cells: progenitor, cytolytic, and terminally exhausted T cells. Cut&Tag-seq was performed on all three subsets for histone marks. H3K4me3, a permissive mark, was enriched in the cytolytic population but then later was lost during the progression of terminal exhaustion in CD8 T cells. A similar pattern was seen in both function-related genes such as TBX21-Tbet and memory-related genes such as Tcf7 and IL7R genes. There is overlap between differentially methylated regions that acquire de novo DNA methylation in exhausted CD8 T cells with genomic regions that lose histone H3K4me3 marks as CD8 T cells become terminally exhausted.

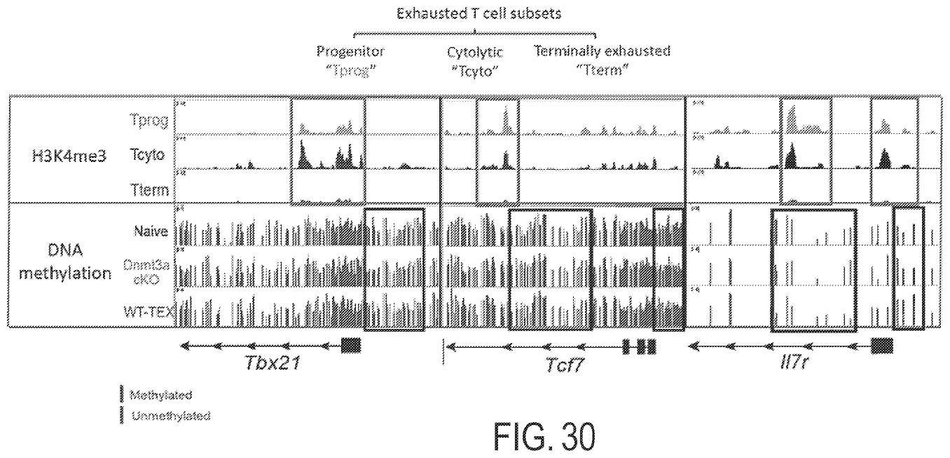

To determine whether changes in histone H3K4me3 modifications correlate with de novo DNA methylation and subsequent chromatin accessibility changes in TEX cell subsets, H3K4me3 profiles of LCMV-specific TEX subsets were integrated with DNA methylation datasets and chromatin landscapes of naive and chronically stimulated Dnmt3a-cKO or WT LCMV-specific CD8 T cells, as well as chromatin accessibility profiles of LCMV-specific Tprog cells or Cxcr5− Tim-3+CD8 T cells isolated from chronically LCMV-infected animals. Tterm cells lose H3K4me3 modifications at effector and memory-related genes, such as Tbx21, Tcf7, Lef1, Ifng, and Myc. These changes in H3K4me3 modifications were coupled with de novo DNA methylation programs and reduced chromatin accessibility in WT TEX cells (FIG. 30) In contrast, chronically stimulated Dnmt3a-deficient cells retained chromatin openness at these loci patterns similar to those in the Tprog and Tcyto subsets.

### Cytolytic TEX Cells Maintain High H3K4Me3 Levels at Effector Genes

H3K4me3 marks were preserved at the promoters of numerous effector genes within Tcyto cells, as evidence by promoters with high H3K4me3 signals in Tcyto vs. Tterm cells exhausted mouse CD8 T cells). However, they were absent in Tterm cells and partially reduced in Tprog cells. (FIG. 20A) This data indicates that Tterm cells lose H3K4me3 marks and undergo de novo DNA methylation, enforcing chromatin inaccessibility and silencing of memory T cell programs.

### In Vitro Model of Human T Cell Dysfunction

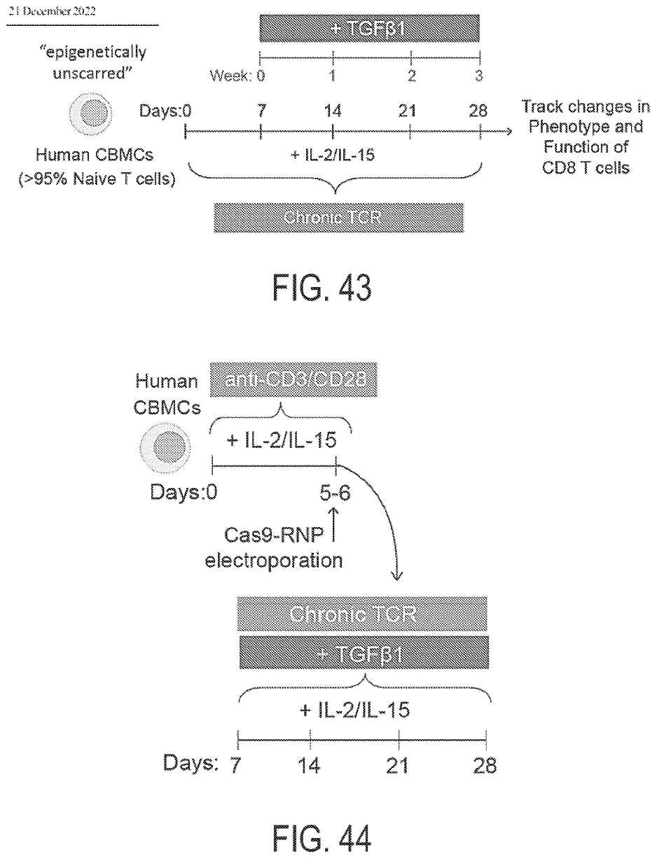

An in vitro test was used to determine whether TGFB1 can drive exhaustion or induce dysfunction on chronically stimulated human CD8 T cells. (FIG. 43) The T cells were stimulated for 28 days with a CD3, but then during effector timepoint on Day 7 TGFB was introduce for up to 3 weeks and the phenotype and function tracked.

Frequency of IFNg and TNFa cytokine producing or functional T cells significantly decreases over the course of the 28 days with the TGFB exposure compared with the Chronic TCR alone, thereby indicating that terminal dysfunction in human CD8 T cells was epigenetically stabilized. Looking at different durations of TGFB, the cells that were exposed to TGFB for longer periods of time showed the most significantly reduced percentages of functional T cells.

### Genetic Targeting of Histone H3K4 Demethylases in Human CD8 T Cells Via CRISPR-Cas9 RNP

The approach for genetic targeting of histone H3K4 demethylases in human CD 8 T cells via CRISPR-Cas9 RNP was performed by ex vivo stimulation of human CBMCs by anti-CD3/CD28, followed by Cas9 editing of target genes on day 7 using Cas9-RNP, and after overnight resting, chronic TCR or chronic TCR+TGF(31 stimulation was continue. (FIG. 44)

Targeting the H3K4 Demethylases KDM5A and KDM5B Enhances the Effector Function of Chronically Stimulated Human CD8 T cells

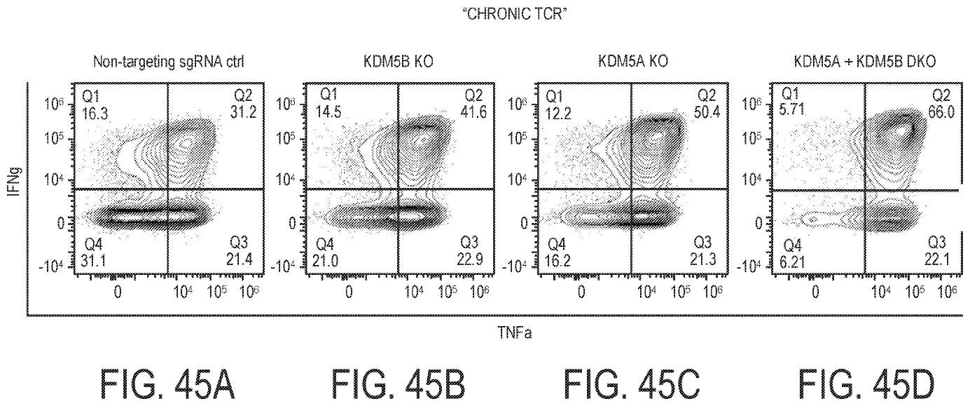

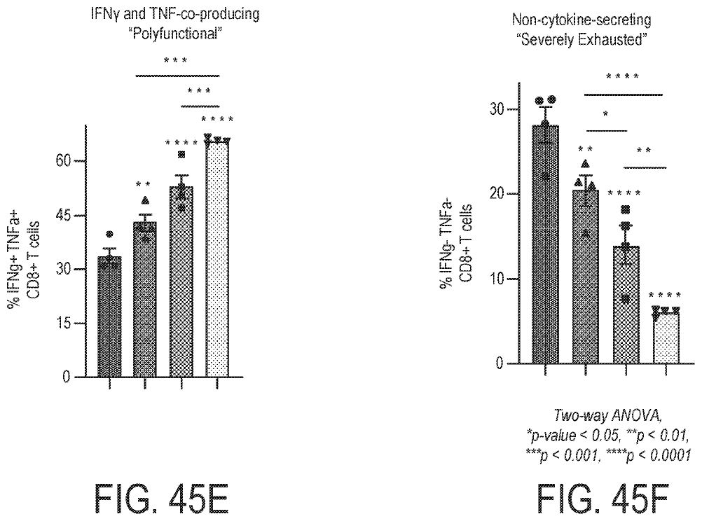

Chronic TCR: To further confirm the cell-intrinsic role of KDM5A and KDM5B in driving T cell dysfunction, the CRISPR-Cas9 gene editing approach was optimized to genetically knock out KDM5B, KDM5A, or both (DKO—double knock out) in activated human CD8 T cells on day 7, followed by chronic TCR or TCR plus TGFB1 stimulation settings. (FIGS. 45A-45F) It was found that KDM5A or KDM5B-edited CD8 T cells show significant improvement in their polyfunctionality while reducing the frequency of the severely exhausted cells that lack IFNG and TNF secretion. DKO CD8 T cells showed the most striking recovery in the effector functions and the least frequency of the severely exhausted cells. This data confirms the cell-intrinsic role of both KDM5A and KDM5B in driving terminal exhaustion in chronically stimulated human CD8 T cells and suggests non-redundant roles of both enzymes in enforcing T cell exhaustion.

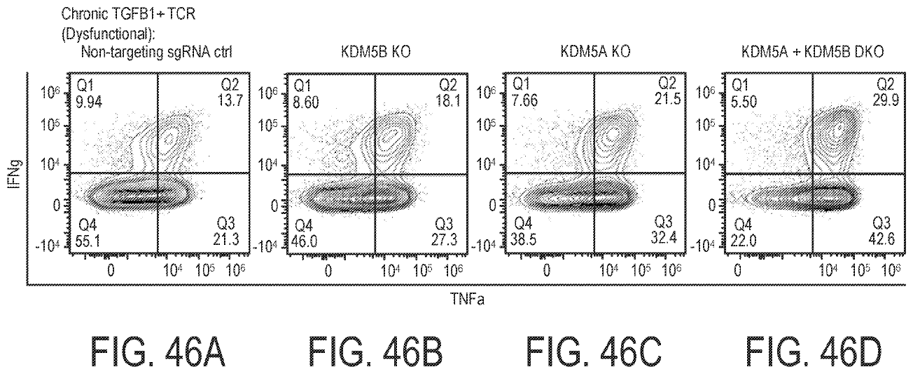

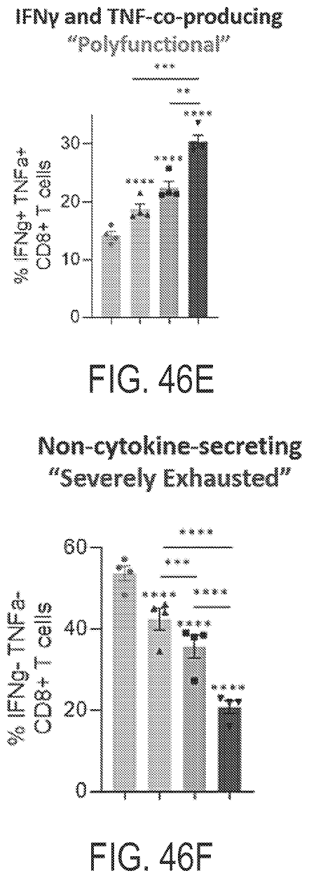

Chronic TGFB1+TCR (Dysfunctional): To further confirm the cell-intrinsic role of KDM5A and KDM5B in driving T cell dysfunction, we optimized the CRISPR-Cas9 gene editing approach to genetically knock out KDM5B, KDM5A, or both (DKO-double knock out) in activated human CD8 T cells on day 7, followed by chronic TCR or TCR plus TGFB1 stimulation settings. (FIGS. 46A-46F) It was found that KDM5A or KDM5B-edited CD8 T cells show significant improvement in their polyfunctionality while reducing the frequency of the severely exhausted cells that lack IFNG and TNF secretion. DKO CD8 T cells showed the most striking recovery in the effector functions and the least frequency of the severely exhausted cells. This data confirms the cell-intrinsic role of both KDM5A and KDM5B in driving terminal exhaustion in chronically stimulated human CD8 T cells and suggests non-redundant roles of both enzymes in enforcing T cell exhaustion.

### Genetic Targeting of KDM5A and KDM5B

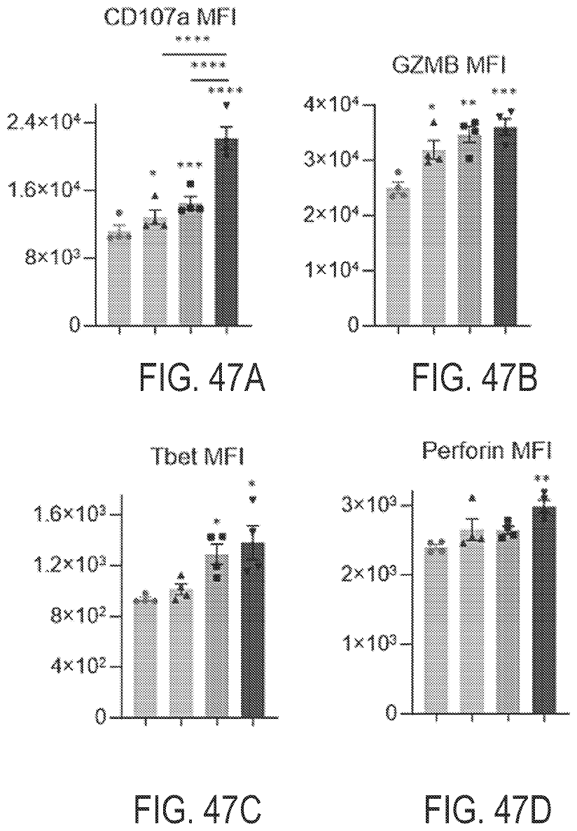

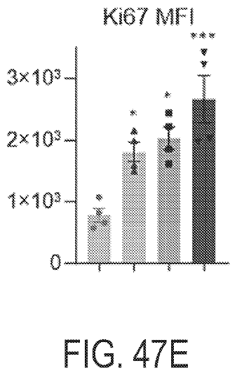

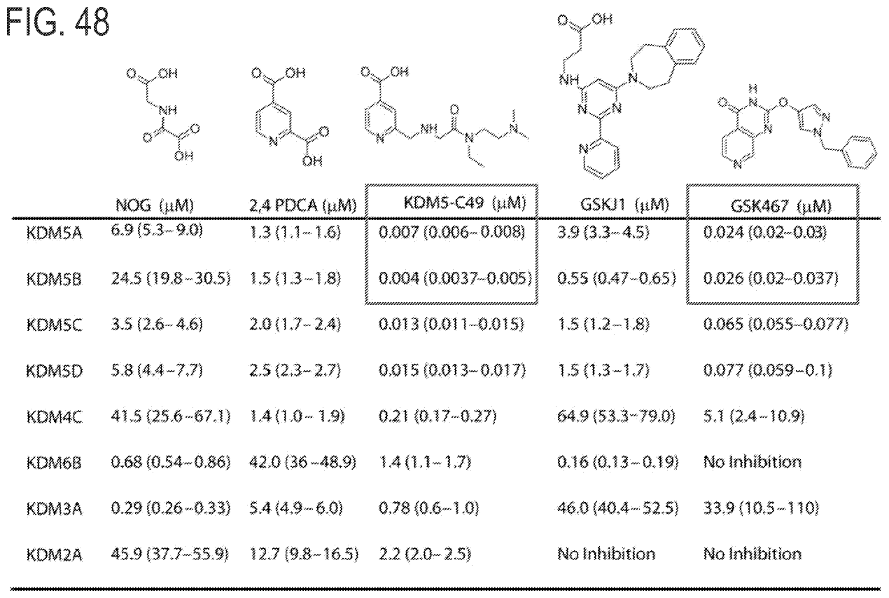

Genetic targeting of KDM5A and KDM5B boosts the cytotoxic and degranulation activities and enhances proliferation capacity of dysfunctional human CD8 T cells, as shown by the protein expression levels of CD107a, GZMB, TBET, and Perforin in CD8+ T cells after PMA/Ionomycin stimulation (day 28). (FIGS. 47A-47D)

### Therapeutic Inhibition of KDM5A B-Mediated H3K4 Demethylation Enhances Effector Functions of Human TEX Cells

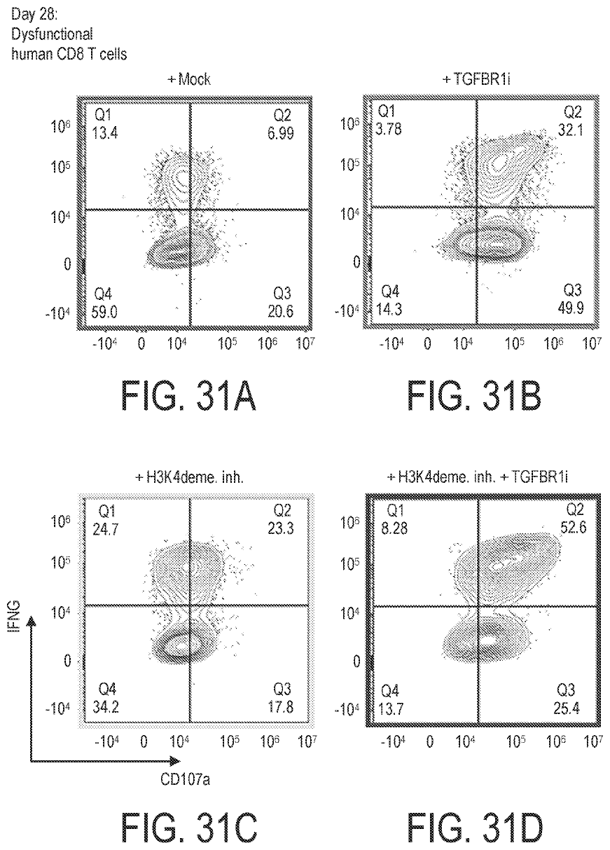

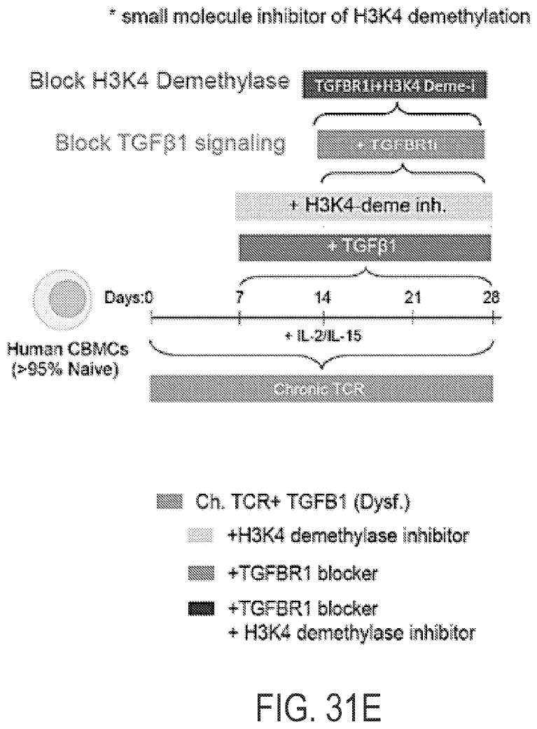

To test the therapeutic efficacy of targeting KDMSA/B in dysfunctional human CD8 T cells, the in vitro model of human CD8 T cell dysfunction was used and chronically TCR plus TGFβ1-stimulated human T cells were treated with the selective inhibitor of KDMSA/B, GSK467 (25 μM), on days 14-28 with or without TGFBR1 blocker. (FIGS. 31A-3E) While TGFβ1 blockade recovered effector functions of chronically stimulated CD8 T cells, KDMSA/B inhibitor treatment further potentiated the reactivation of polyfunctionality and memory expression programs. KDMSA/Bi monotherapy significantly restored effector cytokine secretion and expression of memory-related transcription factors in dysfunctional human CD8 T cells.

### Therapeutic Inhibition of KDM5A B-Mediated H3K4 Demethylation Enhances Effector Functions of Human TEX Cells

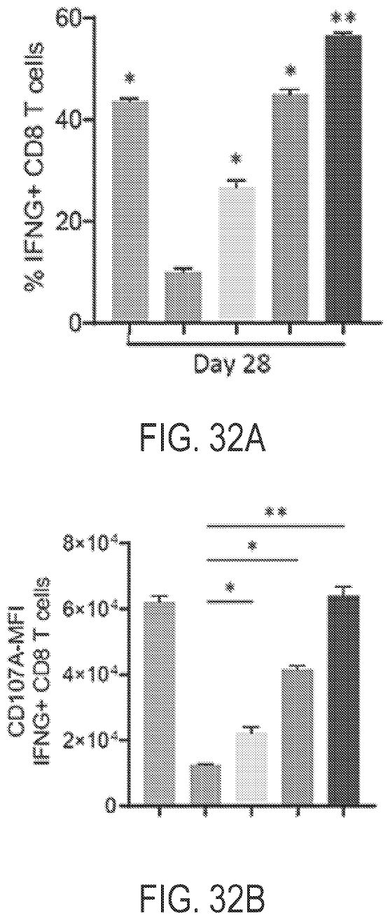

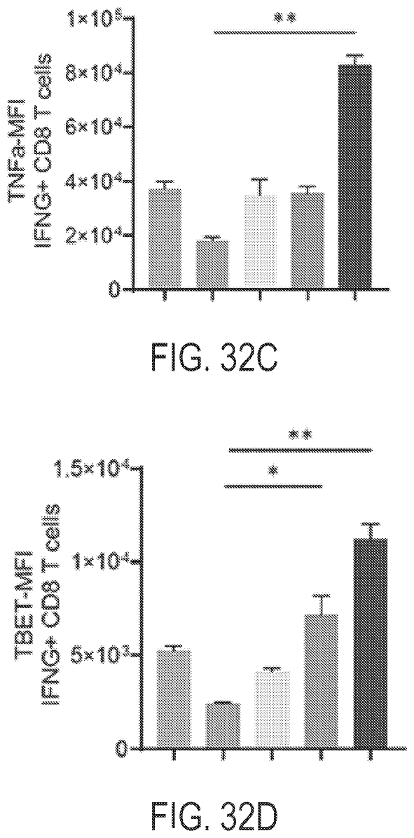

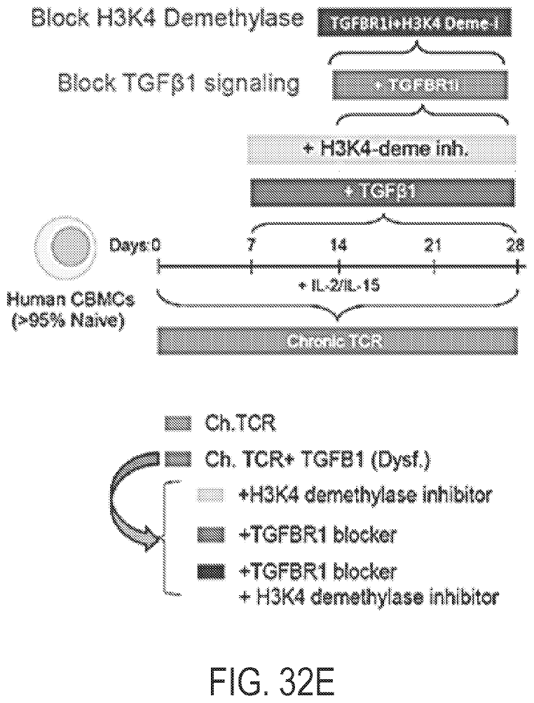

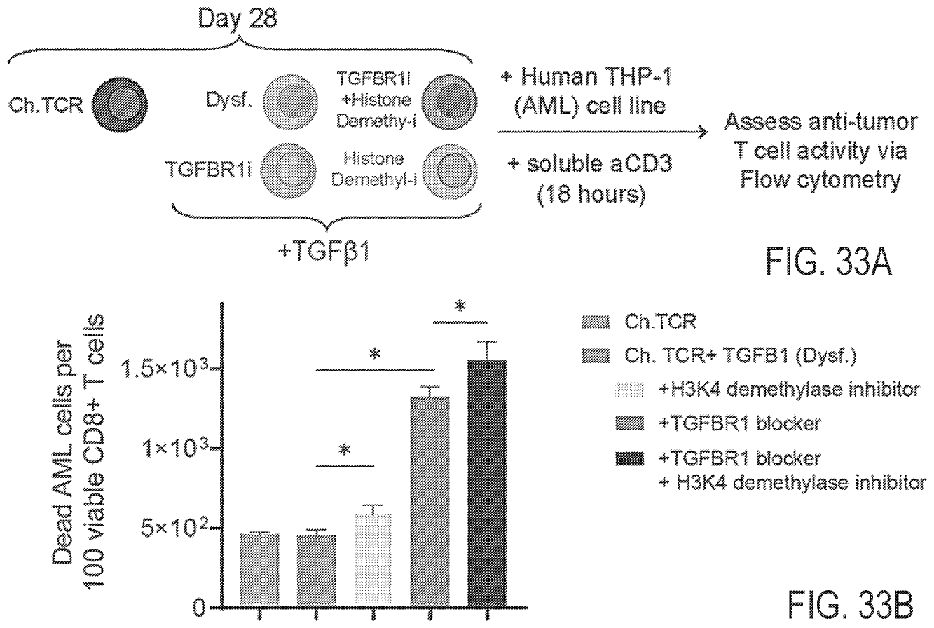

GSK467: To test the therapeutic efficacy of targeting KDM5A/B in dysfunctional human CD8 T cells, the in vitro model of human CD8 T cell dysfunction was employed and chronically TCR plus TGFβ1-stimulated human T cells were treated with the selective inhibitor of KDM5A/B, GSK467 (25 μM), on days 14-28 with or without TGFBR1 blocker. (FIGS. 32A-32E) While TGFβ1 blockade recovered effector functions of chronically stimulated CD8 T cells, KDM5A/B inhibitor treatment further potentiated the reactivation of polyfunctionality and memory expression programs. KDM5A/Bi monotherapy significantly restored effector cytokine secretion and expression of memory-related transcription factors in dysfunctional human CD8 T cells.

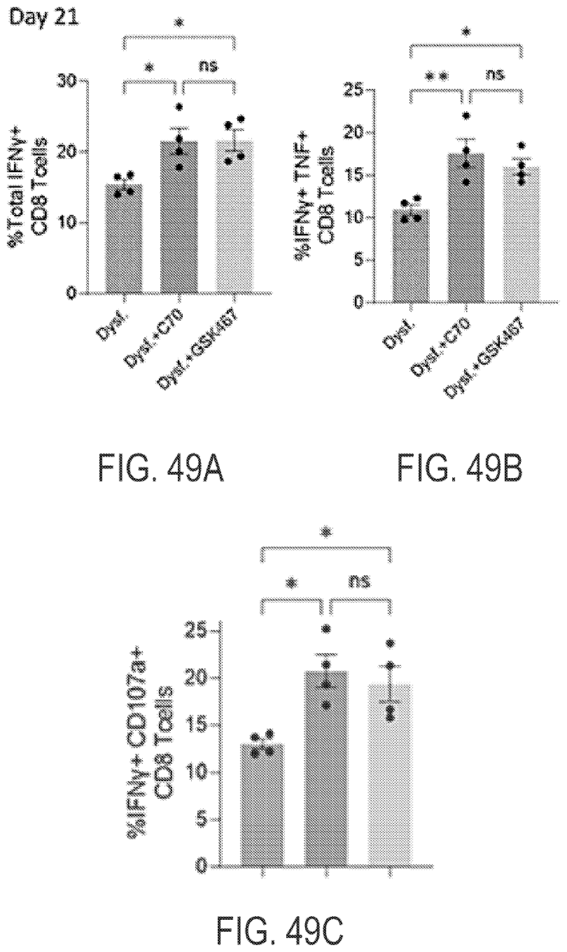

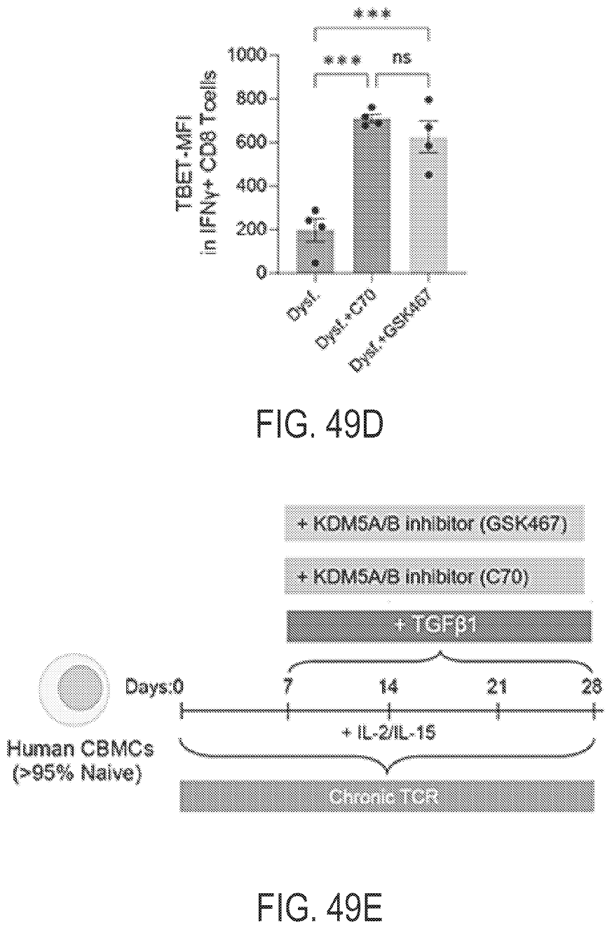

CD70 and/or GSK467: To test the therapeutic efficacy of targeting KDM5A/B in dysfunctional human CD8 T cells, the in vitro model of human CD8 T cell dysfunction was employed and chronically TCR plus TGFβ1-stimulated human T cells were treated with another selective inhibitor of KDM5A/B, C70 compound (25 μM) which is a derivative of KDM5-C49, on days 14-28. KDM5A/B inhibitor treatment using either GSK467 or C70 drugs showed similar activity and significantly enhanced polyfunctionality and memory expression programs in dysfunctional human CD8 T cells. (FIGS. 49A-49E)

Therapeutic Inhibition of KDM1A (LSD1) does not Enhance Effector Functions of Human TEX Cells

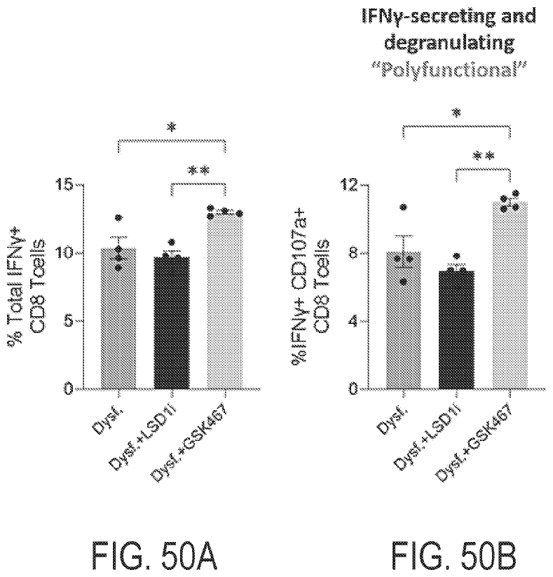

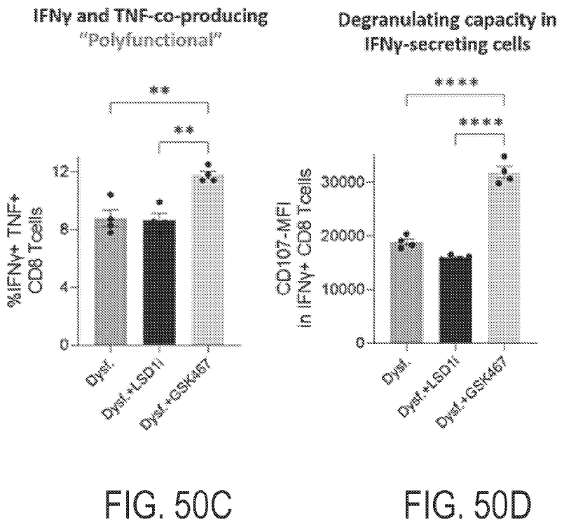

To determine whether the improved effector functions after therapeutic inhibition of KDM5A and KDM5B enzymes can be also attained by inhibiting another H3K4 demethylase KDM1A (LSD1), efficacy of another small molecules inhibitor of KDM1A (LSD1) enzyme (25 uM) was tested and it was found that this treatment didn't improve the effector functions or degranulation activity of dysfunctional human CD8 T cells. (FIGS. 50A-50D)

### Targeting H3K4 Demethylation Reduces De Novo DNA Methylation at Effector Genes

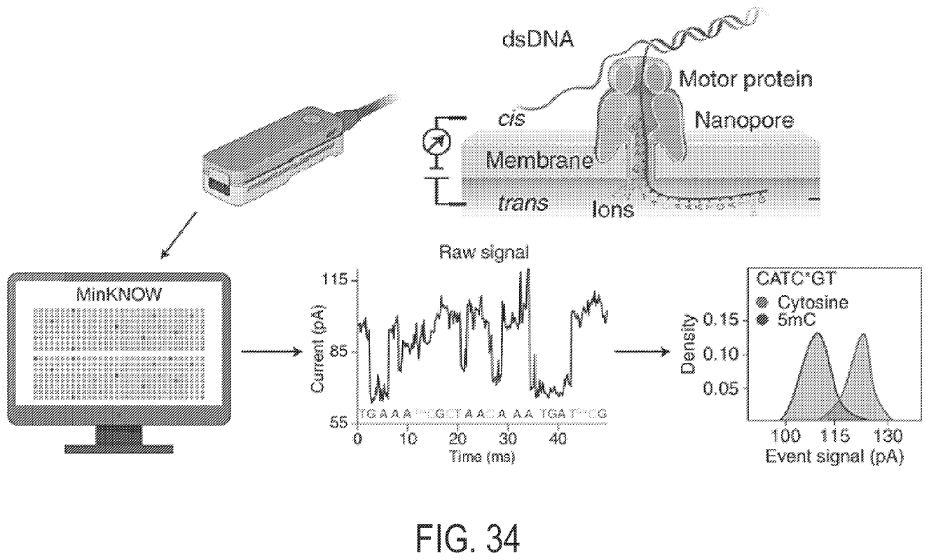

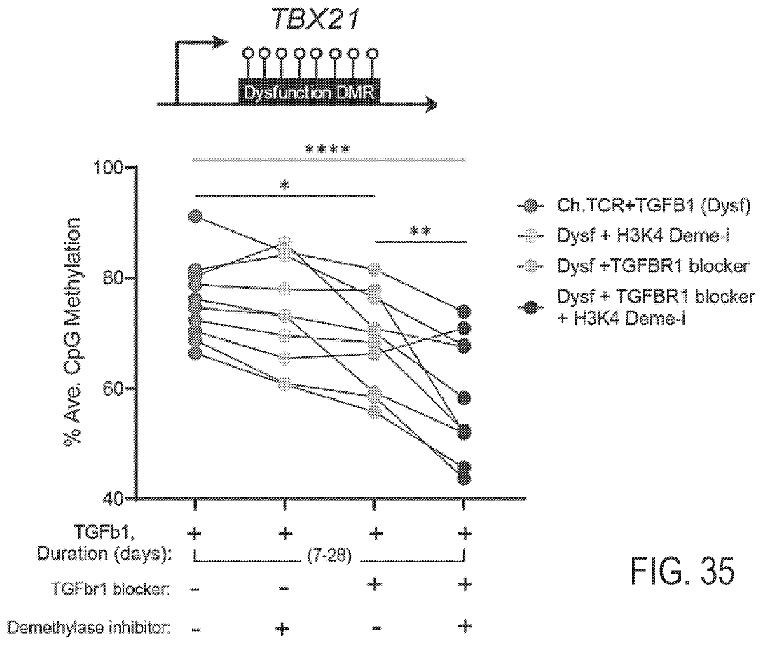

To determine whether targeting H3K4 demethylation can reduce de novo DNA methylation programs at the effector function-related genes in human CD8 T cells, the changes in DNA methylation levels were assessed at the dysfunction-associated differentially methylated region in dysfunctional human CD8 T cells after GSK467 (KDM5A/B inhibitor) with or without TGFBR1 inhibitor (Repsox) treatment. (FIGS. 34 and 35) Using a targeted epigenetic assay optimized by using Oxford Nanopore technology, it was found that therapeutic targeting of H3K4 demethylation significantly reduced the acquisition of de novo DNA methylation marks at the TBX21 locus (that encodes TBET transcription factor which is a master regulator of IFNG secretion and effector function in CD8 T cells). These data indicate that maintaining H3K4 methylation can prevent the process of de novo DNA methylation programming that enforces terminal dysfunction of human CD8 T cells.

### The Impact of Therapeutic Blockade of H3K4 De-Me on TEX Responses to ICB Therapy

The efficacy of KDM5A/B inhibitor (GSK467) therapy was tested during lifelong chronic LCMV infection in animals. This model induces a severe level of T cell exhaustion. (FIG. 14A)

### Therapeutic Blockade of H3K4 De-Me Boosts the Responses of Anti-Viral CD8 T Cells to ICB

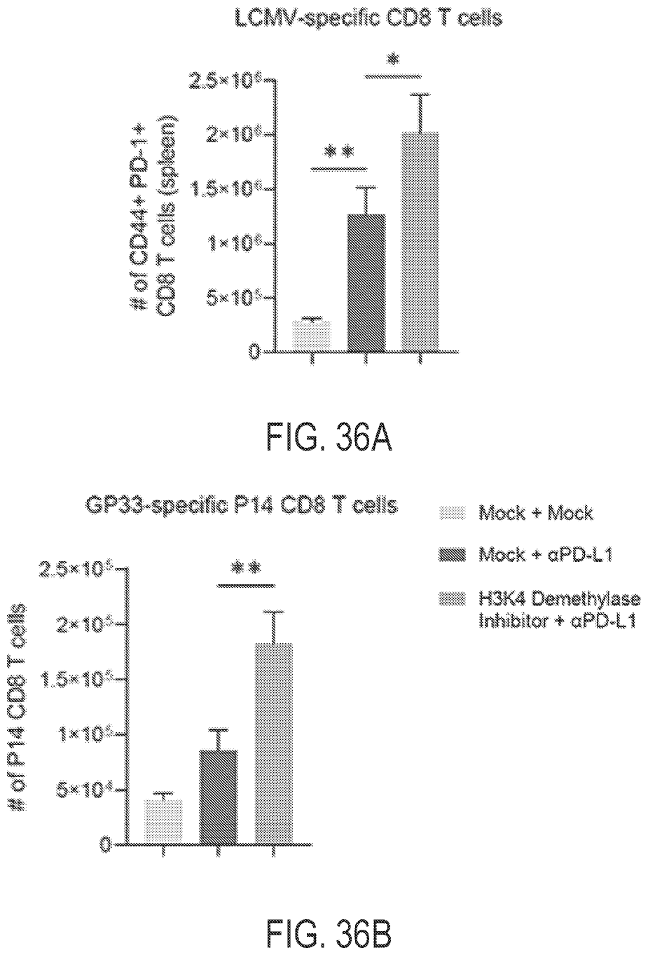

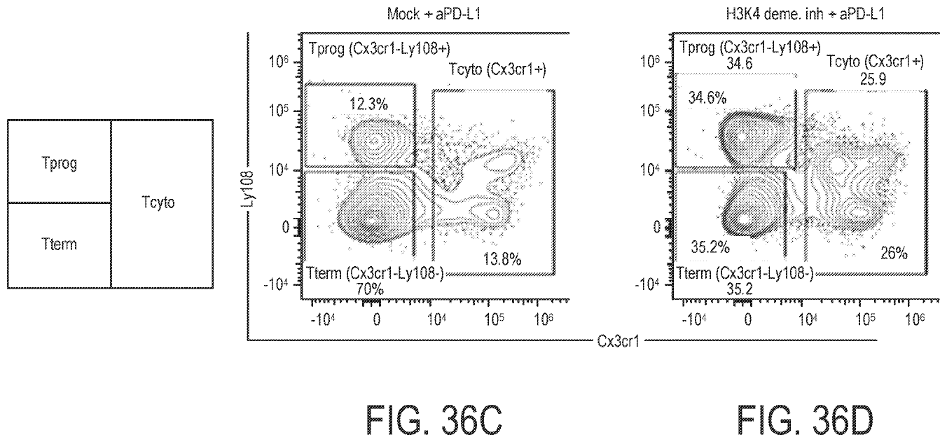

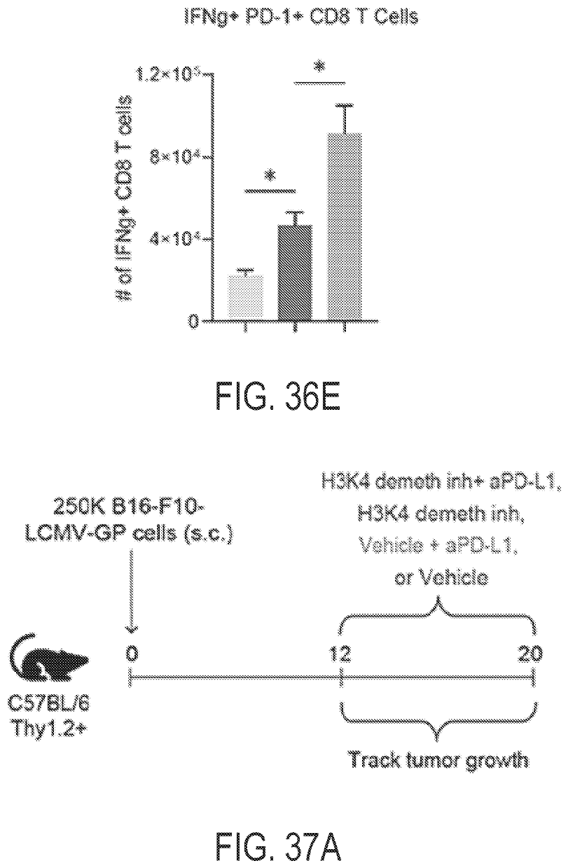

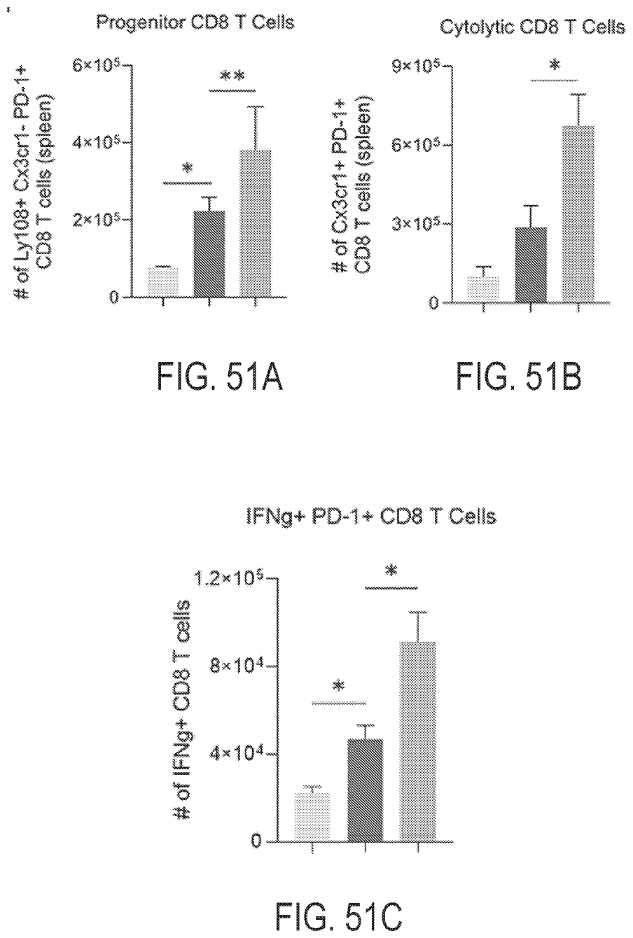

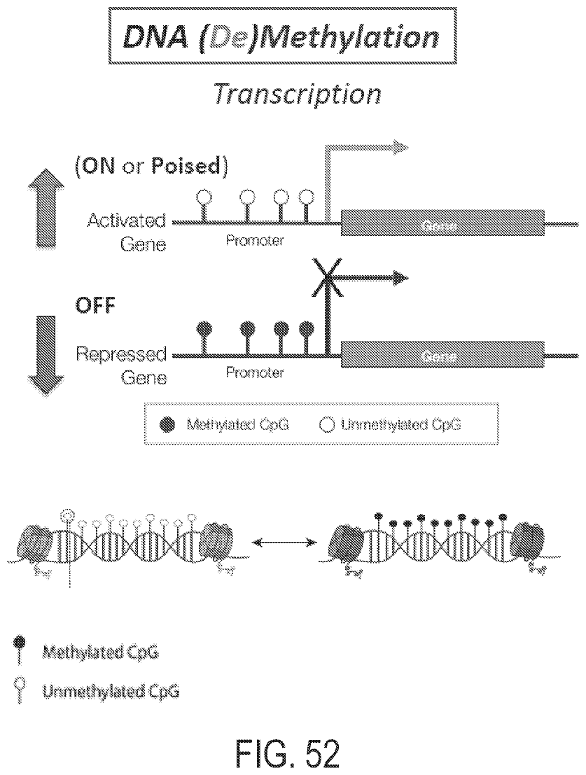

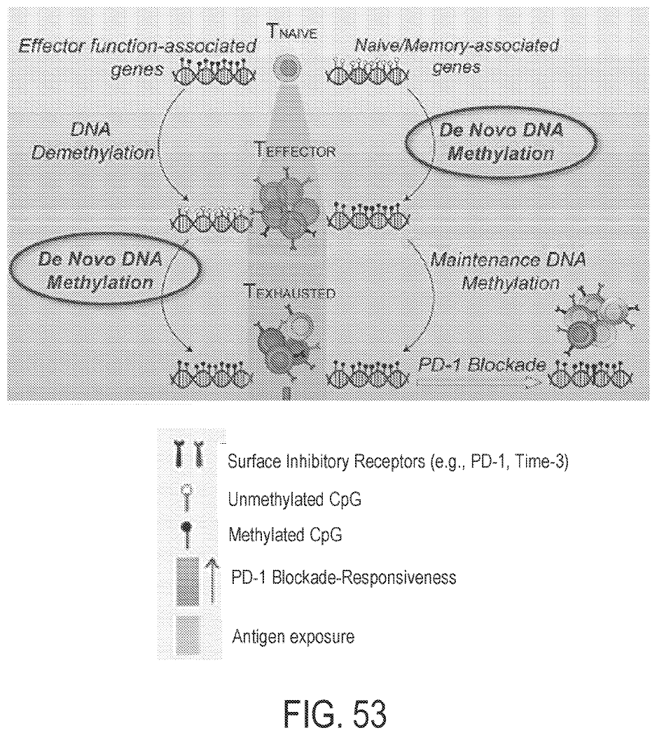

Late GSK467 (KDM5A/Bi) treatment (5 mg/Kg, every 2 days by i.p injection on days 25-33 p.i) enhanced the responses of virus-specific exhausted CD8 T cells to PD-L1 blockade, with significant expansion of both Tprog and Tcyto cells, as well as IFNG-secreting virus-specific CD8 T cells. These findings demonstrate the therapeutic efficacy of KDM5A/B blockade to enhance the rejuvenation of exhausted T cells. (FIGS. 36A-36E, 51A-51C)

### Therapeutic Blockade of H3K4 De-Me Boosts the Responses of Anti-Tumor CD8 T Cells to ICB

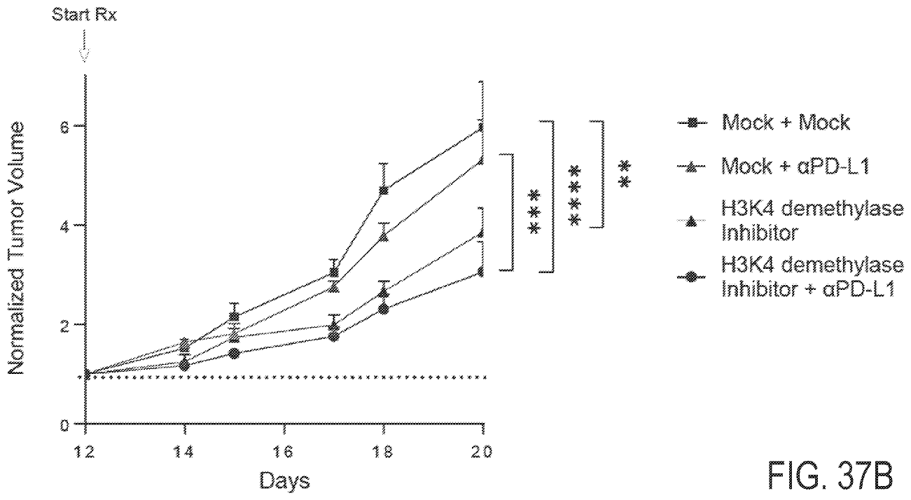

B16-F10 Melanoma Model: The efficacy of KDM5A/B inhibitor (GSK467) therapy was tested using B16-F10 melanoma model that is refractory to immune checkpoint blockade (e.g., anti-PD-L1) therapy. Dose was 5 mg/Kg, every 2 days by i.p injection on days 12-20 post-tumor inoculation. GSK467 monotherapy resulted in significant tumor control which was further potentiated with combination PD-L1 blockade therapy. (FIGS. 37A-37B)

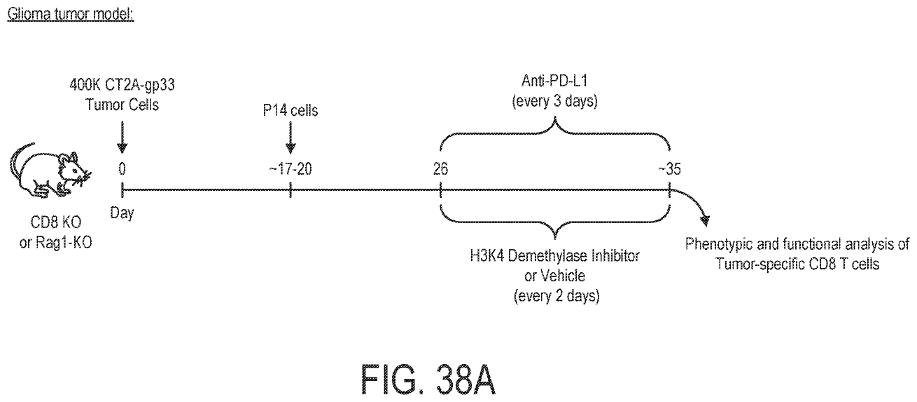

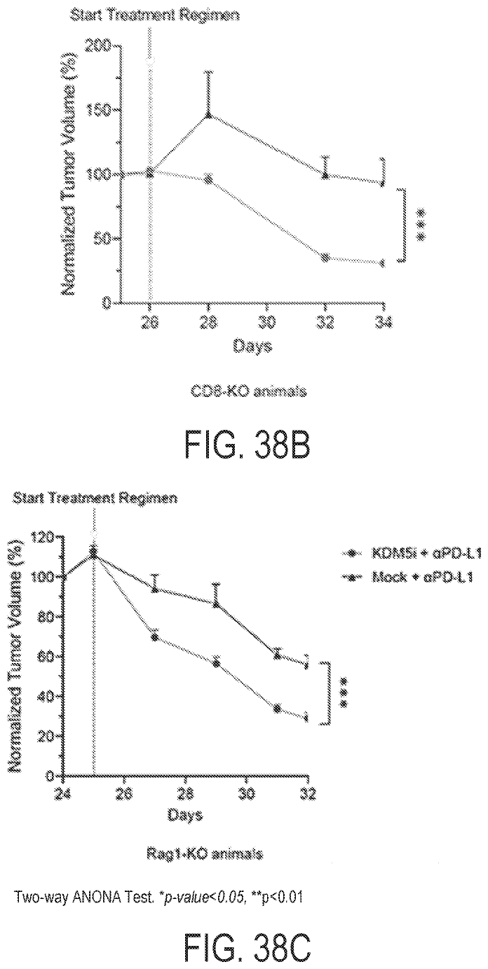

CT2A-gp33 Glioma Model: The efficacy of GSK467 therapy was tested using CT2A-gp33 glioma model with gp33-specific P14 CD8 T cells adoptively transferred on day 17-20. It was found that combined GSK467 plus PD-L1 blockade therapy resulted in significant tumor control using either CD8-KO or Ragi-KO animals. (FIGS. 38A-38C)

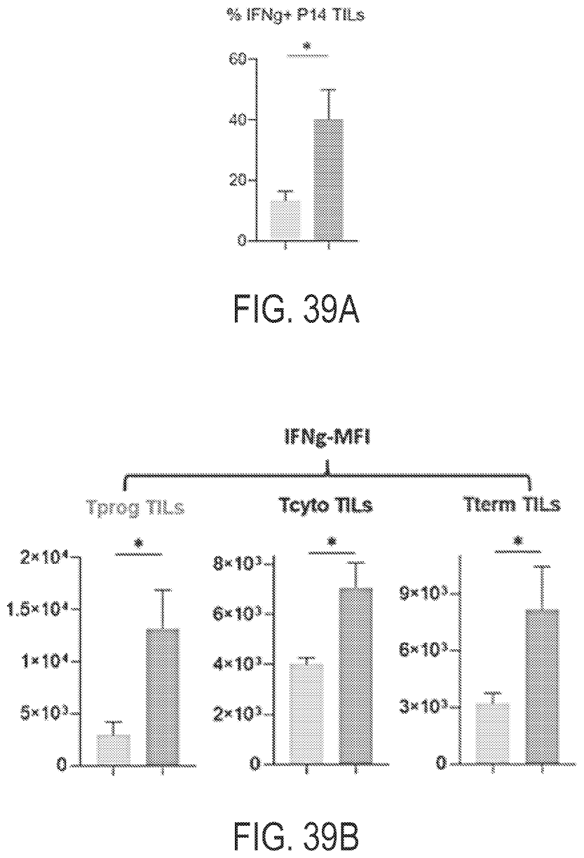

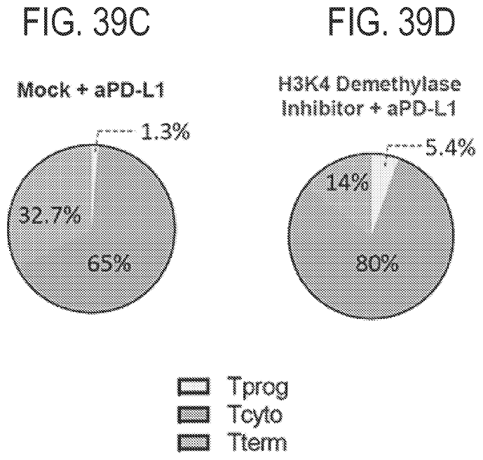

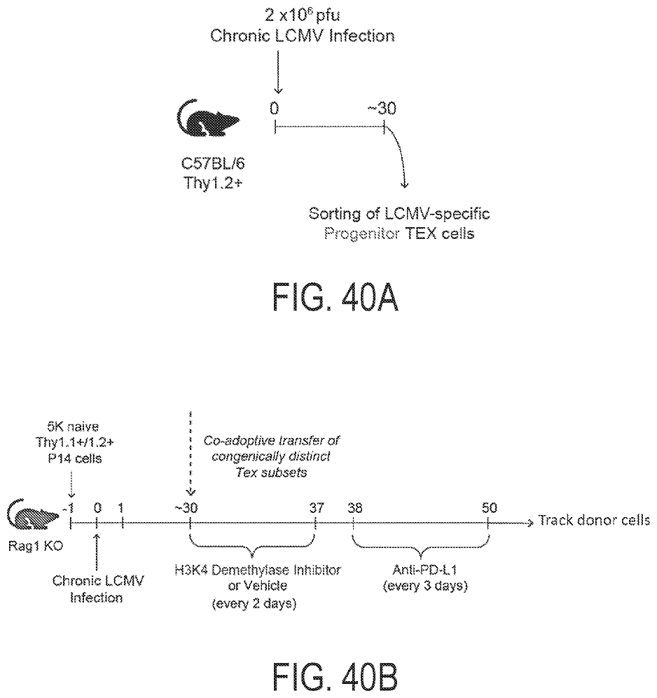

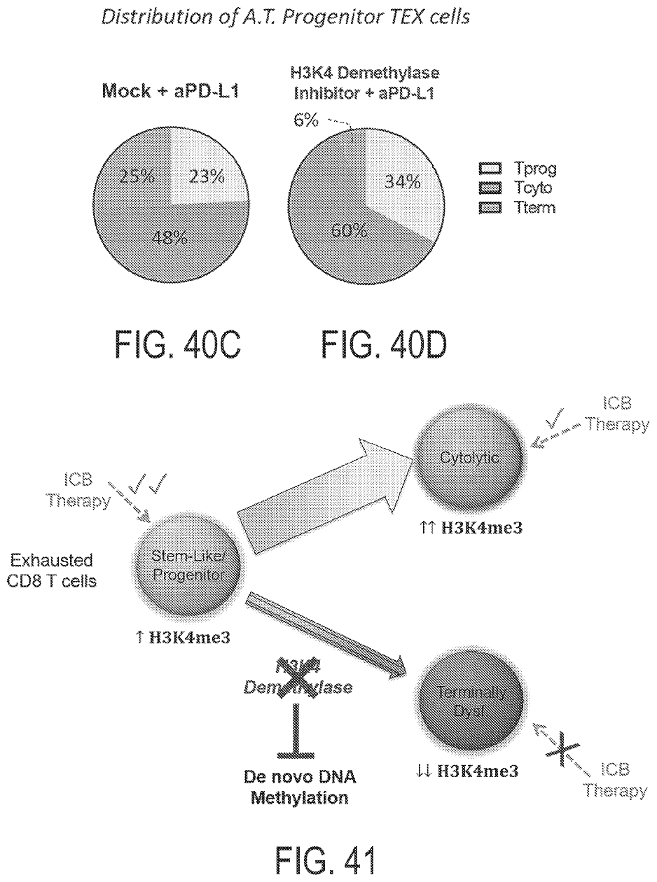

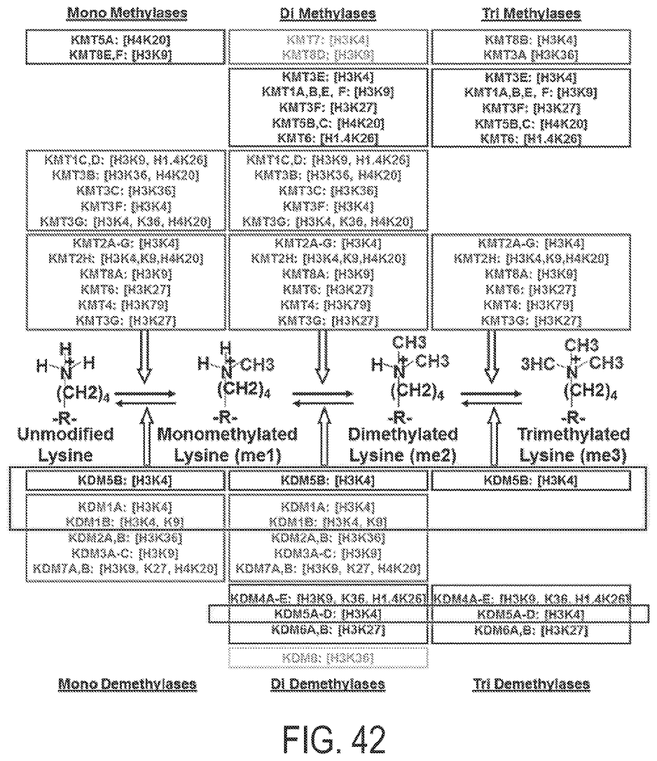

The analysis of tumor-infiltrating lymphocytes revealed that combined GSK467 plus PD-L1 blockade treatment induced significant increase in the quantities of tumor-specific CD8 T cells as well as increased frequencies of IFNG-secreting tumor-specific CD8 T cells. (FIGS. 39A-39D)

To confirm the therapeutic efficacy of GSK467 treatment in different cancer settings, the treatment efficacy was tested in orthotopic model of breast cancer that is refractory to PD-L1 blockade therapy. It was found that combined GSK467 plus PD-L1 blockade therapy resulted in a significant breast tumor control in treated animals compared to PD-L1 blockade only. (FIGS. 16A-16C)

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### REFERENCES

Abdel-Hakeem, M. S. et al. Epigenetic scarring of exhausted T cells hinders memory differentiation upon eliminating chronic antigenic stimulation. Nat Immunol 22, 1008-1019 (2021).
Alvarez-Breckenridge, C. et al. Microenvironmental Landscape of Human Melanoma Brain Metastases in Response to Immune Checkpoint Inhibition. Cancer Immunology Research 10, 996-1012 (2022).
Angelosanto, J. M., Blackburn, S. D., Crawford, A. & Wherry, E. J. Progressive loss of memory T cell potential and commitment to exhaustion during chronic viral infection. J Virol 86, 8161-8170 (2012).
Barber, D. L. et al. Restoring function in exhausted CD8 T cells during chronic viral infection. Nature 439, 682-687 (2006).
Begley, C. G. & Ellis, L. M. Drug development: Raise standards for preclinical cancer research. Nature 483, 531-533 (2012).
Beltra, J., et al. Developmental Relationship of Four Exhausted CD8+ T Cell Subsets Reveals Underlying Transcriptional and Epigenetic Landscape Control Mechanisms. Immunity 52:5, 825-841 (2020).
Bintu, L. et al. Dynamics of epigenetic regulation at the single-cell level. Science 351, 720-724 (2016).
Bird, A. DNA methylation patterns and epigenetic memory. Genes Dev 16, 6-21 (2002).
Black, J. C., Van Rechem, C. & Whetstine, J. R. Histone lysine methylation dynamics: establishment, regulation, and biological impact. Mol Cell 48, 491-507 (2012).
Chen, Y. et al. BATF regulates progenitor to cytolytic effector CD8+ T cell transition during chronic viral infection. Nat Immunol 22, 996-1007 (2021).
Daniel, B. et al. Divergent clonal differentiation trajectories of T cell exhaustion. Nat Immunol (2022) doi:10.1038/s41590-022-01337-5.
Deng, Q. et al. Characteristics of anti-CD19 CAR T cell infusion products associated with efficacy and toxicity in patients with large B cell lymphomas. Nat Med 26, 1878-1887 (2020).
Ford, B. R. & Poholek, A. C. Regulation and Immunotherapeutic Targeting of the Epigenome in Exhausted CD8 T Cell Responses. The Journal of Immunology 210, 869-879 (2023).
Gadepalli, V. S., Ozer, H. G., Yilmaz, A. S., Pietrzak, M. & Webb, A. BISR-RNAseq: an efficient and scalable RNAseq analysis workflow with interactive report generation. BMC Bioinformatics 20, 670 (2019).
Getzler, A. J. et al. Ml11 pioneers histone H3K4me3 deposition and promotes formation of CD8+T stem cell memory. bioRxiv 2023.01.18.524461 (2023) doi:10.1101/2023.01.18.524461.
Ghoneim, H. E. et al. De Novo Epigenetic Programs Inhibit PD-1 Blockade-Mediated T Cell Rejuvenation. Cell 170, 142-157.e19 (2017).
Ghoneim, H. E., Zamora, A. E., Thomas, P. G. & Youngblood, B. A. Cell-Intrinsic Barriers of T Cell-Based Immunotherapy. Trends Mol Med 22, 1000-1011, (2016).
Giles, J. R. et al. Shared and distinct biological circuits in effector, memory and exhausted CD8+ T cells revealed by temporal single-cell transcriptomics and epigenetics. Nat Immunol (2022) doi:10.1038/s41590-022-01338-4.
Good, C. R. et al. An NK-like CAR T cell transition in CAR T cell dysfunction. Cell 184, 6081-6100.e26 (2021).
Guo, X. et al. Structural insight into autoinhibition and histone H3-induced activation of DNMT3A. Nature 517, 640-644 (2015).
Hudson, W. H. et al. Proliferating Transitory T Cells with an Effector-like Transcriptional Signature Emerge from PD-1+ Stem-like CD8+ T Cells during Chronic Infection. Immunity 51, 1043-1058.e4 (2019).
Im, S. J. et al. Defining CD8+ T cells that provide the proliferative burst after PD-1 therapy. Nature 537, 417-421 (2016).
Janssen, S. M., Lorincz, M. C. Interplay between chromatin marks in development and disease. Nat Rev Genet 23, 137-153 (2022).
Johansson, C. et al. Structural analysis of human KDM5B guides histone demethylase inhibitor development. Nat Chem Biol 12, 539-545 (2016).
Kasmani, M. Y. et al. Clonal lineage tracing reveals mechanisms skewing CD8+ T cell fate decisions in chronic infection. J Exp Med 220, e20220679 (2023).
Kaya-Okur, H. S. et al. CUT&Tag for efficient epigenomic profiling of small samples and single cells. Nat Commun 10, 1930 (2019).
Kim, D., Langmead, B. & Salzberg, S. L. HISAT: a fast spliced aligner with low memory requirements. Nat Methods 12, 357-360 (2015).
Langmead, B. & Salzberg, S. L. Fast gapped-read alignment with Bowtie 2. Nat Methods 9, 357-359 (2012).
Li, B.-Z. et al. Histone tails regulate DNA methylation by allosterically activating de novo methyltransferase. Cell Res 21, 1172-1181 (2011).
Liao, Y., Smyth, G. K. & Shi, W. feature Counts: an efficient general purpose program for assigning sequence reads to genomic features. Bioinformatics 30, 923-930 (2014).
Long, A. H. et al. 4-1BB costimulation ameliorates T cell exhaustion induced by tonic signaling of chimeric antigen receptors. Nat Med 21, 581-590 (2015).
McLane, L. M., Abdel-Hakeem, M. S. & Wherry, E. J. CD8 T Cell Exhaustion During Chronic Viral Infection and Cancer. Annu Rev Immunol 37, 457-495 (2019).
Meers, M. P., Tenenbaum, D. & Henikoff, S. Peak calling by Sparse Enrichment Analysis for CUT&RUN chromatin profiling. Epigenetics & Chromatin 12, 42 (2019).
Milner, J. J. et al. Runx3 programs CD8+ T cell residency in non-lymphoid tissues and tumours. Nature
Mombaerts, P. et al. RAG-1-deficient mice have no mature B and T lymphocytes. Cell 68, 869-877 (1992).
Nakashima, H. et al. Modeling tumor immunity of mouse glioblastoma by exhausted CD8+ T cells. Sci Rep 8, 208 (2018).
Nat Immunol 18, 573-582 (2017).
Pauken, K. E. et al. Epigenetic stability of exhausted T cells limits durability of reinvigoration by PD-1 blockade. Science 354, 1160-1165 (2016).
Peng, V. et al. Whole-genome profiling of DNA methylation and hydroxymethylation identifies distinct regulatory programs among innate lymphocytes. Nat Immunol23, 619-631 (2022).
Philip, M. et al. Chromatin states define tumour-specific T cell dysfunction and reprogramming. Nature 545, 452-456 (2017).
Pircher, H., Burki, K., Lang, R., Hengartner, H. & Zinkernagel, R. M. Tolerance induction in double specific T-cell receptor transgenic mice varies with antigen. Nature 342, 559-561 (1989).
Ramirez, F. et al. deepTools2: a next generation web server for deep-sequencing data analysis. Nucleic Acids Res 44, W160-165 (2016).
Saadey, A. A. et al. Rebalancing TGFβ1/BMP signals in exhausted T cells unlocks responsiveness to immune checkpoint blockade therapy. Nat Immunol (2022) doi:10.1038/s41590-022-01384-y.
Saadey, A. A., Yousif, A. & Ghoneim, H. E. Chapter Seven—Epigenetic programming of the immune responses in cancer. in Cancer Immunology and Immunotherapy (eds. Amiji, M. M. & Milane, L. S.) 197-235 (Academic Press, 2022). doi:10.1016/B978-0-12-823397-9.00007-7.
Sakamoto, Y. et al. Long-read whole-genome methylation patterning using enzymatic base conversion and nanopore sequencing. Nucleic Acids Res 49, e81 (2021).
Savas, P. et al. Single-cell profiling of breast cancer T cells reveals a tissue-resident memory subset associated with improved prognosis. Nat Med 24, 986-993 (2018).
Scharer C D, Bally A P, Gandham B, Boss J M. Cutting Edge: Chromatin Accessibility Programs CD8 T Cell Memory. J Immunol. 2017 Mar. 15; 198(6):2238-2243. doi: 10.4049/jimmunol.1602086. Epub 2017 Feb 8. PMID: 28179496; PMCID: PMC5341694.
Scott-Browne, J. P. et al. Dynamic Changes in Chromatin Accessibility Occur in CD8+ T Cells Responding to Viral Infection. Immunity 45, 1327-1340 (2016).
Sen, D. R. et al. The epigenetic landscape of T cell exhaustion. Science 354, 1165-1169 (2016).
Sharifi-Zarchi, A. et al. DNA methylation regulates discrimination of enhancers from promoters through a H3K4me1-H3K4me3 seesaw mechanism. BMC Genomics 18, 964 (2017).
Sharma, P. & Allison, J. P. Dissecting the mechanisms of immune checkpoint therapy. Nat Rev Immunol 20, 75-76 (2020).
Simpson, J. T. et al. Detecting DNA cytosine methylation using nanopore sequencing. Nat Methods 14, 407-410 (2017).
Sudmeier, L. J. et al. Distinct phenotypic states and spatial distribution of CD8+ T cell clonotypes in human brain metastases. Cell Rep Med 3, 100620 (2022).
Tooley, K. A., Escobar, G. & Anderson, A. C. Spatial determinants of CD8+ T cell differentiation in cancer. Trends Cancer 8, 642-654 (2022).
Tsuda, S. & Pipkin, M. E. Transcriptional Control of Cell Fate Determination in Antigen-Experienced CD8 T Cells. Cold Spring Harb Perspect Biol 14, a037945 (2022).
Utzschneider, D. T. et al. T cells maintain an exhausted phenotype after antigen withdrawal and population reexpansion. Nat Immunol 14, 603-610 (2013).
Wang, Y. Single-molecule long-read sequencing reveals the chromatin basis of gene expression. Genome Res 29, 1329-1342, (2019).
Weber, E. W. et al. Transient rest restores functionality in exhausted CAR-T cells through epigenetic remodeling. Science 372, eaba1786 (2021).
Weber, M. et al. Distribution, silencing potential and evolutionary impact of promoter DNA methylation in the human genome. Nat Genet 39, 457-466 (2007).
Welsh, R. M. & Seedhom, M. O. Lymphocytic choriomeningitis virus (LCMV): propagation, quantitation, and storage. Curr Protoc Microbiol Chapter 15, Unit 15A.1 (2008).
Wu, H. et al. Detection of differentially methylated regions from whole-genome bisulfite sequencing data without replicates. Nucleic Acids Res 43, e141 (2015).
Xi, Y. & Li, W. BSMAP: whole genome bisulfite sequence MAPping program. BMC Bioinformatics 10, 232 (2009).
Yang, Y. et al. TCR engagement negatively affects CD8 but not CD4 CAR T cell expansion and leukemic clearance. Sci Transl Med 9, eaag1209 (2017).
Yates, K. B. et al. Epigenetic scars of CD8+ T cell exhaustion persist after cure of chronic infection in humans. Nat Immunol22, 1020-1029 (2021).
Yenyuwadee, S. et al. Immune cellular components and signaling pathways in the tumor microenvironment. Semin Cancer Biol 86, 187-201 (2022).
Yousif, A. & Ghoneim, H. E. T cell exhaustion-a memory locked behind scars. Nat Immunol 22, 938-940 (2021).
Yu, B. et al. Epigenetic landscapes reveal transcription factors that regulate CD8+ T cell differentiation.
Zhang, SM., Cai, W. L., Liu, X. et al. KDM5B promotes immune evasion by recruiting SETDB1 to silence retroelements. Nature 598, 682-687 (2021).
Zhang, Y. et al. Chromatin methylation activity of Dnmt3a and Dnmt3a/3L is guided by interaction of the ADD domain with the histone H3 tail. Nucleic Acids Res 38, 4246-4253 (2010).
Zhang, Y. et al. Model-based analysis of ChIP-Seq (MACS). Genome Biol 9, R137 (2008).
Zhu, Z. et al. FOXP1 and KLF2 reciprocally regulate checkpoints of stem-like to effector transition in CAR T cells. Nat Immunol 25, 117-128 (2024).

What is claimed is:

1. A method of preventing T-cell terminal exhaustion comprising contacting the T-cell with a histone demethylase inhibitor.

2. The method of claim 1, comprising contacting the T-cell in vivo in a subject in need thereof.

3. The method of claim 1, comprising contacting the T-cell ex vivo.

4. The method of claim 1, wherein the histone demethylase inhibitor comprises an inhibitor of H3K4me3.

5. The method of claim 4, wherein H3K4me3 comprises KDM5A enzyme, KDM5B enzyme, or any combination thereof.

6. The method of claim 4, wherein H3K4me3 comprises KDM1A enzyme, KDM1B enzyme, or any combination thereof

7. The method any one of claims 4-5, wherein the inhibitor of H3K4me3 comprises JIB-04, CPI455, QC6352, AS8351, KDOAM-25 citrate, KDM5A-IN-1, KDM5-IN-1, TK-129, YUKA1, JQKD82 trihydrochloride, CPI-455 hydrochloride, KDM5-C49, JQKD82, GSK467, C70, or any combination thereof.

8. The method of any one of claims 4 and 6, the inhibitor of H3K4me3 comprises ladademstat, GSK2879552, tranylcypromine hemisulfate, pulrodemstat benzenesulfonate, seclidemstat, bomedemstat dihydrochloride, GSK 690 hydrochloride, S2101, INCB059872 dihydrochloride, HDAC6-IN-3, LSD1-IN-30, CBB1007 trihydrochloride, INCB059872, CBB1007 hydrochloride, LSD1-IN-6, LSD1/HDAC6-IN-1, MC2652, LSD1-UM-109, S1427, LSD1-IN-23, LSD1/2-IN-4, NCL1, cis-4-Br-2,5-F2-PCPA, KDM1/CDK1-IN-1, LSD1-IN-26, LSD1-IN-21, LSD1-IN-18, LSD1-IN-25, arborinine, LSD1-IN-22, LSD1-IN-19, LSD1/2-IN-3, tranylcypromine-d5 hydrochloride, LSD1/ER-IN-1, CBB1003 hydrochloride, GSK467, C70, or any combination thereof.

9. The method of any one of claims 4-8, wherein the inhibitor of H3K4me3 comprises GSK467, C70, or any combination thereof.

10. The method of any one of claims 1-9, wherein the T-cell is a CD8 T-cell.

11. A method of improving CAR-T therapy effectiveness comprising:
(1) contacting a genetically engineered CAR-T-cell with a histone demethylase inhibitor; and
(2) administering the CAR-T-cell to a subject in need thereof.

12. The method of claim 11, wherein the genetically engineered CAR-T-cell is contacted with the histone demethylase inhibitor ex vivo.

13. The method of claim 11, wherein the genetically engineered CAR-T-cell is contacted with the histone demethylase inhibitor in vivo in a subject in need thereof.

14. A method of improving CAR-T therapy effectiveness comprising:
(1) administering a genetically engineered CAR-T-cell to a subject in need thereof, and
(2) administering a histone demethylase inhibitor to a subject in need thereof.

15. The method of any one of claims 11-14, wherein the histone demethylase inhibitor comprise an H3K4me3 inhibitor.

16. The method of claim 15, wherein H3K4me3 comprises KDM5A enzyme, KDM5B enzyme, or any combination thereof.

17. The method of claim 15, H3K4me3 comprises KDM1A enzyme, KDM1B enzyme, or any combination thereof

18. The method of claim 16, wherein the inhibitor of H3K4me3 comprises JIB-04, CPI455, QC6352, AS8351, KDOAM-25 citrate, KDM5A-IN-1, KDM5-IN-1, TK-129, YUKA1, JQKD82 trihydrochloride, CPI-455 hydrochloride, KDM5-C49, JQKD82, GSK467, C70, or any combination thereof.

19. The method of claim 17, the inhibitor of H3K4me3 comprises ladademstat, GSK2879552, tranylcypromine hemisulfate, pulrodemstat benzenesulfonate, seclidemstat, bomedemstat dihydrochloride, GSK 690 hydrochloride, S2101, INCB059872 dihydrochloride, HDAC6-IN-3, LSD1-IN-30, CBB1007 trihydrochloride, INCB059872, CBB1007 hydrochloride, LSD1-IN-6, LSD1/HDAC6-IN-1, MC2652, LSD1-UM-109, S1427, LSD1-IN-23, LSD1/2-IN-4, NCL1, cis-4-Br-2,5-F2-PCPA, KDM1/CDK1-IN-1, LSD1-IN-26, LSD1-IN-21, LSD1-IN-18, LSD1-IN-25, arborinine, LSD1-IN-22, LSD1-IN-19, LSD1/2-IN-3, tranylcypromine-d5 hydrochloride, LSD1/ER-IN-1, CBB1003 hydrochloride, GSK467, C70, or any combination thereof.

20. The method of any one of claims 15-19, wherein the inhibitor of H3K4me3 comprises GSK467, C70, or any combination thereof

21. The method of any one of claims 11-20, wherein the T-cell is a CD8 T-cell.

22. A method of treating cancer comprising administering to a subject in need thereof, a histone demethylase inhibitor.

23. A method of treating a chronic virus infection comprising administering to a subject in need thereof, a histone demethylase inhibitor.

24. A method of improving immune activity in a subject in need thereof, comprising administering to the subject a histone demethylase inhibitor.

25. A method of treating a disease or disorder comprising administering to a subject in need thereof a histone demethylase inhibitor.

26. The method of any one of claims 22-25, further comprising administering an immune checkpoint inhibitor.

27. The method of any one of claims 22-26, wherein administering to the subject in need thereof a histone demethylase inhibitor comprises contacting a T-cell ex vivo and administering to the subject the T-cell.

28. The method of any one of claims 22-26, wherein administering to the subject in need thereof a histone demethylase inhibitor comprises contacting a T-cell in vivo in the subject in need thereof.

29. The method of any one of claims 22-28, wherein the histone demethylase inhibitor comprises an inhibitor of H3K4me3.

30. The method of any one of claims 22 and 26-28, wherein the cancer comprises breast adenocarcinoma or glioma.

31. The method of any one of claims 22-30, wherein the immune checkpoint inhibitor inhibits PD-1, PD-L1, CTLA-4, LAG3, or any combination thereof.

32. The method of any one of claims 26-31, wherein the immune checkpoint inhibitor inhibiting PD-1 comprises nivolumab, pembrolizumab, cemiplimab, dostarlimab, retifanlimab, toripalimab, or any combination thereof.

33. The method of any one of claims 26-32, wherein the immune checkpoint inhibitor inhibiting PD-L1 comprises atezolizumab, avelumab, durvalumab, or any combination thereof.

34. The method of any one of claims 26-33, wherein the immune checkpoint inhibitor inhibiting CTLA-4 comprises ipilimumab, tremelimumab, or any combination thereof.

35. The method of any one of claims 26-34, wherein the immune checkpoint inhibitor inhibiting LAG-3 comprises eftilagimod alpha, relatlimab, favezolimab, fianlimab, or any combination thereof.

36. The method of any one of claims 29-35, wherein H3K4me3 comprises KDM5A enzyme, KDM5B enzyme, or any combination thereof.

37. The method of any one of claims 29-35, wherein H3K4me3 comprises KDM1A enzyme, KDM1B enzyme, or any combination thereof

38. The method of claim 36, wherein the inhibitor of H3K4me3 comprises JIB-04, CPI455, QC6352, AS8351, KDOAM-25 citrate, KDM5A-IN-1, KDM5-IN-1, TK-129, YUKA1, JQKD82 trihydrochloride, CPI-455 hydrochloride, KDM5-C49, JQKD82, GSK467, C70, or any combination thereof.

39. The method of claim 37, the inhibitor of H3K4me3 comprises ladademstat, GSK2879552, tranylcypromine hemisulfate, pulrodemstat benzenesulfonate, seclidemstat, bomedemstat dihydrochloride, GSK 690 hydrochloride, S2101, INCB059872 dihydrochloride, HDAC6-IN-3, LSD1-IN-30, CBB1007 trihydrochloride, INCB059872, CBB1007 hydrochloride, LSD1-IN-6, LSD1/HDAC6-IN-1, MC2652, LSD1-UM-109, S1427, LSD1-IN-23, LSD1/2-IN-4, NCL1, cis-4-Br-2,5-F2-PCPA, KDM1/CDK1-IN-1, LSD1-IN-26, LSD1-IN-21, LSD1-IN-18, LSD1-IN-25, arborinine, LSD1-IN-22, LSD1-IN-19, LSD1/2-IN-3, tranylcypromine-d5 hydrochloride, LSD1/ER-IN-1, CBB1003 hydrochloride, GSK467, C70, or any combination thereof.

40. The method of any one of claims 29-39, wherein the inhibitor of H3K4me3 comprises GSK467, C70, or any combination thereof.

41. The method of any one of claims 27-40, wherein the T-cell is a CD8 T-cell

42. The method of any one of claims 23 and 26-41, wherein the chronic virus infection comprises acquired immune deficiency syndrome (AIDS), AIDS-related complexes, chronic hepatitis, long COVID, subacute sclerosing panencephalitis (chronic measles encephalitis), chronic papovavirus encephalitis (progressive multifocal leukoencephalopathy), spongioform encephalopathies, herpesvirus-induced diseases, neoplasis, or any combination thereof.
